(19) United States
(12) Patent Application Publication
(10) Pub. No.: US 2024/0361326 A1
(43) Pub. Date: Oct. 31, 2024

(54) EXOSOMAL LIPIDS AND METABOLITES FOR THE EARLY DETECTION OF HEPATOCELLULAR CARCINOMA

(71) Applicant: BOARD OF REGENTS, THE UNIVERSITY OF TEXAS SYSTEM, Austin, TX (US)

(72) Inventor: Laura BERETTA, Bellaire, TX (US)

(21) Appl. No.: 18/577,761

(22) PCT Filed: Jul. 8, 2022
(86) PCT No.: PCT/US2022/036467

§ 371 Date: Jan. 9, 2024

(51) Int. Cl. G01N 33/574; G01N 33/68; G01N 33/92

(52) U.S. Cl. CPC G01N 33/57488; G01N 33/6848; G01N 33/92

(57) ABSTRACT

A method of detecting hepatocellular carcinoma (HCC) biomarkers in exosomes isolated from a blood sample obtained from a human subject is provided, by detecting in the sample two or more exosomal HCC biomarkers. The subject can be a healthy patient, a patient suspected of having a liver disorder, a patient previously diagnosed with liver cirrhosis, or a patient previously diagnosed with liver fibrosis.

### CROSS-REFERENCE TO RELATED APPLICATIONS

This application claims the benefit of and priority to U.S. Provisional Patent Application Ser. No. 63/220,074, filed Jul. 9, 2021, the content of which is incorporated herein by reference in its entirety.

### BACKGROUND

Liver cancer is the second most common cause of cancer-related deaths worldwide. Hepatocellular carcinoma (HCC) represents 70-90% of all liver cancers. HCC has a poor prognosis with 5 year survival rate below 20% and surveillance in high-risk subjects is a promising approach to reduce mortality. Professional societies such as the American Association for the Study of Liver Diseases, recommend HCC surveillance in patients with cirrhosis. Surveillance in cirrhotic patients with ultrasound and serum alphafetoprotein (AFP) is commonly used. However, HCC surveillance remains underused in clinical practice, leading to high proportion of late stage detection and both ultrasound and AFP lack sensitivity and specificity. Thus, there is a need for innovative approaches to promote HCC surveillance in patients with cirrhosis and for novel biomarkers to complement imaging.

Exosomes are membrane bound nanovesicles (30-150 nm) that contain various molecular components such as proteins, lipids, and nucleic acids. They can be found in body fluids such as plasma, ascites, and urine. Exosomes represent potentially valuable noninvasive diagnostic biomarkers, therapeutic targets, and drug carriers. They are recognized as key players in cancer growth, metastasis, and angiogenesis and are therefore important mediators of cancer progression. Exosomes have also been shown to modulate inflammation and downregulate antitumor immunity. While still in its infancy, the clinical application of exosomes to cancer detection has shown some promise. With the rise of interest in exosomes and “omics” studies, databases such as Vesiclepedia have surfaced to track studies and their downstream analyses such as mRNA, miRNA, protein and lipids. The most common type of downstream analysis is proteomics followed by mRNA profiling. More recent “omics” studies have also investigated the lipid and metabolite content of exosomes.

There is thus a need for new efficient and accurate methods for detecting early stage HCC in patients, such as patients with liver cirrhosis or other HCC risk factors. The present disclosure satisfies this need and provides other advantages as well.

### BRIEF SUMMARY

The present disclosure provides methods and compositions for detecting hepatocellular carcinoma (HCC) in subjects, such as subjects suspected of having a liver disorder. The present methods and compositions are based on the detection of HCC biomarkers in exosomes isolated from biological samples obtained from a subject, e.g., from a blood sample. Accordingly, in one aspect, the present disclosure provides a method of detecting hepatocellular carcinoma biomarkers in exosomes isolated from a sample, the method comprising detecting in the exosomes one or more hepatocellular carcinoma biomarkers, wherein the one or more hepatocellular carcinoma biomarkers comprise one or more hepatocellular carcinoma biomarkers listed in any one or more of Table 1, Table 2, Table 3, Table 4, or Table 7A, any one or more two-way combinations of biomarkers listed in Table 4, Table 5, or Table 7B, and/or any one or more three-way combinations of hepatocellular carcinoma biomarkers listed in Table 7C, and wherein the sample is a blood sample obtained from a human subject.

In some embodiments, the hepatocellular carcinoma biomarkers comprise one or more of the lipid classes sphingosine (SPH), sulfatide (ST), and lysophosphatidylserine (LysoPS). In some embodiments, the sphingosine lipid class comprises SPH(t18:0), the sulfatide lipid class comprises ST(d18:1/20:2), and/or the lysophosphatidylserine lipid class comprises LysoPS(34:1). In some embodiments, the hepatocellular carcinoma biomarkers comprise a lipid species selected from the group consisting of PC(18:1/24:2), PE(20:Op/20:3), and LysoPC(18:2). In some embodiments, the hepatocellular carcinoma biomarkers further comprise alpha-fetoprotein. In some embodiments, the subject is a patient previously diagnosed with liver cirrhosis. In some embodiments, the liver cirrhosis is advanced liver cirrhosis.

In some embodiments, the detecting comprises detection by gas chromatography, mass spectroscopy, gas chromatography-mass spectrometry or liquid chromatograph-mass spectrometry. In some embodiments, the mass spectrometry is ultra-high resolution mass spectrometry. In some embodiments, 2, 3, 4, 5, 6, 7, 8, 9, 10, or more biomarkers are detected in the exosomes. In some embodiments, the blood sample is a plasma sample. In some embodiments, the exosomes are isolated from the sample by fractionation. In some embodiments, the detecting comprises determining the concentration of the one or more biomarkers in the exosomes. In some embodiments, the method further comprises comparing the concentration of the one or more biomarkers in the exosomes isolated from the blood sample from the subject to a control level, wherein the control level corresponds to the concentration of the one or more biomarkers in exosomes isolated from a blood sample from a healthy individual without hepatocellular carcinoma. In some embodiments, the method further comprises treating the subject with an anti-hepatocellular carcinoma treatment based on a detection of a difference in the concentration of the one or more biomarkers in the exosomes isolated from the blood sample from the subject relative to the control level.

In some embodiments, the difference comprises an elevated level of SPH, a reduced level of ST, and/or an elevated level of LysoPS in the exosomes isolated from the blood sample from the subject relative to the control level. In some embodiments, the subject is undergoing treatment for hepatocellular carcinoma, and wherein the sample comprises at least two samples obtained at different time points during the treatment. In some embodiments, the subject is undergoing treatment for hepatocellular carcinoma, and wherein the sample comprises at least one sample obtained at a time point prior to start of the treatment, and at least one sample obtained at a time point subsequent to the start of the treatment. In some embodiments, the treatment comprises one or more of a drug treatment, a radiation treatment or a surgical treatment.

In another aspect, the present disclosure provides a method of generating a report containing information on results of detection of hepatocellular carcinoma biomarkers in exosomes, comprising: detecting in the exosomes one or more hepatocellular carcinoma biomarkers; and, generating the report, wherein the one or more hepatocellular carcinoma biomarkers comprise one or more hepatocellular carcinoma biomarkers listed in any one or more of Table 1, Table 2, Table 3, Table 4, or Table 7A, any one or more two-way combinations of biomarkers listed in Table 4, Table 5, or Table 7B, and/or any one or more three-way combinations of hepatocellular carcinoma biomarkers listed in Table 7C, and wherein the exosomes have been isolated from a blood sample obtained from a subject, and wherein the report is useful for diagnosing hepatocellular carcinoma in the subject. In some embodiments, the one or more hepatocellular carcinoma biomarkers are one or more of the lipid classes sphingosine (SPH), sulfatide (ST), and lysophosphatidylserine (LysoPS).

In another aspect, the present disclosure provides a system for detecting hepatocellular carcinoma biomarkers in exosomes, comprising a station for analyzing the exosomes by ultra high resolution mass spectrometry to detect one or more hepatocellular carcinoma biomarkers in the exosomes, wherein the one or more hepatocellular carcinoma biomarkers comprise one or more hepatocellular carcinoma biomarkers listed in any one or more of Table 1, Table 2, Table 3, Table 4, or Table 7A, any one or more two-way combinations of biomarkers listed in Table 4, Table 5, or Table 7B, and/or any one or more three-way combinations of hepatocellular carcinoma biomarkers listed in Table 7C, and wherein the exosomes have been isolated from a blood sample from a subject.

In some embodiments, the one or more hepatocellular carcinoma biomarkers are one or more of the lipid classes sphingosine (SPH), sulfatide (ST), and lysophosphatidylserine (LysoPS). In some embodiments, the system further comprises a station for generating a report containing information on results of the analyzing.

A better understanding of the nature and advantages of embodiments of the present disclosure may be gained with reference to the following detailed description and the accompanying drawings.

### BRIEF DESCRIPTION OF THE DRAWINGS

FIGS. 1A-1B show Principal Component analyses (PCAs) of plasma and exosomal lipid profiles from HCC and non-HCC patients shows four distinct clusters according to certain aspects of this disclosure. PCAs were performed using Euclidean distances, based on (FIG. 1A) log 10 relative abundance of detected lipid classes; and (FIG. 1B) log 10 relative abundance of detected lipid species. Ellipses were drawn using the SD of point scores. p values were calculated using Permutational Multivariate Analysis of Variance (PERMANOVA) test.

FIGS. 2A-2C are volcano and forest plots of lipid classes in HCC exosomes vs non-HCC exosomes according to certain aspects of this disclosure. (FIG. 2A) Log10 of Benjamini-Hochberg (BH) adjusted p values and Log2 of fold change (FC) for all lipid classes were fitted onto the plot. Names are shown for lipid classes enriched (FC>1.5, BH p<0.05) or depleted (FC<−1.5, BH p<0.05) in HCC exosomes vs non-HCC exosomes. Firth logistic regression was performed to assess the associations, (FIG. 2B) between high (Tertile T3) abundance of enriched lipid classes named in (FIG. 2A) and HCC, and (FIG. 2C) between depletion of the lipid classes named in (FIG. 2A) and HCC. OR: odds ratio; AOR: OR adjusted for age, gender and BMI. AcHexSiE: acylGlcSitosterol ester; CerP: ceramide phosphate; CerPE: ceramide phosphoethanolamine; CL: cardiolipin; DLCL: dilysocardiolipin; FA: fatty acid; GD1a: ganglioside; Hex1Cer: hexosylceramide; Hex2Cer: dihexosylceramide; LysoPS: lysophosphatidylserine; OAHFA: (O-acyl)-1-hydroxy fatty acid; PG: phosphatidylglycerol; SPH: sphingosine; ST: sulfatide.

FIG. 3 is a PCA of plasma and exosomal metabolite profiles from HCC and non-HCC patients according to certain aspects of this disclosure. PCA was performed using Euclidean distances, based on log 10 relative abundance of detected metabolites. Ellipses were drawn using the SD of point scores. p values were calculated using Permutational Multivariate Analysis of Variance (PERMANOVA) test.

FIGS. 4A-4B are volcano and forest plots of metabolites in HCC exosomes vs non-HCC exosomes according to certain aspects of this disclosure. (FIG. 4A) Log10 of Benjamini-Hochberg (BH) adjusted p values and Log2 of fold change (FC) for all metabolites were fitted onto a volcano plot. Metabolites upregulated (FC>1.5, BH p<0.05) in HCC exosomes vs non-HCC exosomes are labeled on the graph. (FIG. 4B) Firth logistic regression was performed to assess the association between high (Tertile T3) abundance of the metabolites named in (A) and HCC. OR: odds ratios; AOR: OR adjusted for age, gender and BMI. LysoPC: lysophosphatidylcholine; GCA: glycocholic acid; TCA: taurocholic acid; TDCA: taurodeoxycholic acid.

FIG. 5 is a plot illustrating KEGG pathways associated with enriched or depleted lipids and metabolites in HCC exosomes compared to non-HCC exosomes according to certain aspects of this disclosure. These pathways were identified using a combination of MetaboAnalyst 4.0.2 and LIPEA. Each dot represents one pathway and the size of dot depicts the metabolite and lipid counts in our data associated with the pathway.

FIGS. 6A-6C are plots of receiver operating characteristic (ROC) curves for selected exosomal lipids and metabolites in the diagnosis of HCC according to certain aspects of this disclosure. True positive rate (sensitivity) is plotted in function of the false positive rate (100-specificity). Areas under the curve (AUC) values are shown for: (FIG. 6A) AFP; (FIG. 6B) SPH and SPH+AFP (upper left), ST and ST+AFP (middle left), LysoPS and LysoPS+AFP (lower left), PC(18:1/24:2) and PC(18:1/24:2)+AFP (upper right), ST(d18:1/20:2) and ST(d18:1/20:2)+AFP (middle right), PE(20:Op/20:3) and PE(20:0p/20:3)+AFP (lower right); (FIG. 6C) LysoPC(18:2(9,12))+AFP (upper left), GCA+AFP (lower left); TDCA+AFP (upper right); and cholesterol sulfate+AFP (lower right). AFP: alpha-fetoprotein; GCA: glycocholic acid; LysoPC: lysophosphatidylcholine; LysoPS: lysophosphatidylserine; PC: phosphatidylcholine; PE: phosphatidylethanolamine; SPH: sphingosine; ST, sulfatide; TDCA, taurodeoxycholic acid.

FIG. 7 is scatter plots of selected metabolites enriched in HCC exosomes compared to non-HCC exosomes according to certain aspects of this disclosure. Mean and SEM are shown and significance was determined by Mann-Whitney U test adjusted by Benjamini-Hochberg method. LysoPC: lysophosphatidylcholine. The Y-axis values are: 2.5×10⁸ (max), 2×10⁸, 1.5×10⁸, 1×10⁸, 5×10⁷, 0 (min) for cholesterol sulfate; 8×10⁸ (max), 6×10⁸, 4×10⁸, 2×10⁸, 0 (min) for glycocholic acid; 4×10⁹ (max), 3×10⁹, 2×10⁹, 1×10⁹, 0 (min) for LysoPC(18:2); 4×10⁸ (max), 3×10⁸, 2×10⁸, 1×10⁸, 0 (min) for taurocholic acid; and 2×10⁹ (max), 1.5×10⁹, 1×10⁹, 5×10⁸, 0 (min) for taurodeoxycholic acid.

### DETAILED DESCRIPTION

The present disclosure provides methods and compositions for detecting hepatocellular carcinoma (HCC) in biological samples from subjects, and in particular in exosomes isolated from biological samples such as plasma samples. The present methods and compositions involve biomarkers identified from the analysis of biological samples, e.g., exosomes from plasma, from patients with known HCC. In particular embodiments, the methods are used to detect HCC in subjects with liver cirrhosis or otherwise at risk of developing HCC. As such, the methods allow the detection of HCC in such subjects at the earliest possible stage, permitting more effective treatment. The markers comprise different molecular classes or species, e.g., lipid classes or species, which can be used alone or in any combination to detect HCC in a subject.

### I. Subjects and Samples

The present methods and compositions can be used to detect HCC in a subject, e.g., a subject with one or more symptoms of HCC or with liver cirrhosis. In various embodiments, the subject may be an adult of any age, a child, or an adolescent. The subject may be male or female. In particular embodiments, the subject is a human.

“Hepatocellular carcinoma” or “HCC” refers to the most common type of primary liver cancer. As used herein, HCC can refer to HCC of any stage, e.g., stage 0, stage A, stage B, stage C and stage D of the Barcelona Clinic Liver Cancer classification. HCC as used herein encompasses all types of HCC, including fibrolamellar, psueoglandular, pleomorphic, and clear cell types. HCC as used herein can encompass any growth pattern, including single large tumor, multiple tumors, or poorly defined tumors with infiltrative growth. In some embodiments, the present methods are used to screen for or detect HCC, e.g., early stage HCC, in a subject with a risk factor for HCC, including, but not limited to, cirrhosis, fibrosis, viral hepatitis (e.g., hepatitis B or C), exposure or consumption of one or more toxins (e.g., alcohol, aflatoxin, excessive iron as in hemochromatosis, pyrrolizidine alkaloids), one or more metabolic conditions (e.g., obesity, diabetes, nonalcoholic steatohepatitis), or congenital conditions such as alpha 1-antitrypsin deficiency. In particular embodiments, the methods are used to screen for HCC in subjects with liver cirrhosis, particularly advanced cirrhosis.

The subject may have one or more symptoms of HCC. A non-limiting list of symptoms includes nausea, loss of appetite, weight loss, fatigue, weakness, jaundice, swelling in the abdomen and/or legs, bruising or bleeding, white chalky stools, fever, abdominal pain, and others. The symptoms can be mild, moderate, or severe. In some embodiments, the subject may be considered at risk for developing HCC, even in the absence of symptoms. For example, the subject may have one or more risk factors such as a history of hepatitis B or C, of excessive alcohol consumption, obesity, diabetes, anabolic steroid use, iron storage disease, elevated consumption of aflatoxin, liver cirrhosis, liver fibrosis, or others. In particular embodiments, the subject has liver cirrhosis. The cirrhosis can be at any stage, e.g., early, intermediate, or advanced cirrhosis. In particular embodiments, the subject has advanced liver cirrhosis, and the methods are used to detect the appearance of HCC as early as possible. Nevertheless, an indication of HCC using the present methods can indicate any stage of HCC. In particular embodiments, the HCC that is detected is early stage HCC.

To assess the HCC biomarker status of the patient, a biological sample is obtained from the subject. In some embodiments, the biological sample is a blood sample. In particular embodiments, the blood sample is plasma. In other embodiments, the blood sample is serum or whole blood. Other suitable samples include urine, ascites, seminal fluid, vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, amniotic fluid, bile, gastric juice, lymph, mucus, pericardial fluid, peritoneal fluid, pleural fluid, pus, saliva, sebum, serous fluid, sputum, sweat, tears, and others. Generally, any biological sample that comprises exosomes can be used. The sample can be obtained from the subject using conventional techniques known in the art.

In particular embodiments, exosomes are purified from the sample, e.g., using fractionation, and the biomarkers are detected in the exosomes. For example, in some embodiments, plasma is obtained from a subject and subjected to serial centrifugation, e.g., at 2,000 g and 10,000 g for 30 and 45 minutes, respectfully, to remove any cellular debris. Subsequently the plasma is ultracentrifuged, e.g., at 150,000 g at 4° C. for two hours. In some embodiments, the pellet is washed, e.g., with PBS, and then centrifuged again at 150,000 g for 2 hours. The resulting pellet can then be frozen and stored, e.g., at −80° C., for subsequent lipidomic and/or metabolomic assessment.

### II. Selection of Biomarkers

The presence of HCC in a subject is determined by detecting levels of HCC biomarkers, e.g., exosome HCC biomarkers, in a biological sample. As used herein, a “biomarker” refers to a molecule whose level in a biological sample, e.g., a blood sample such as a plasma sample, is correlated with the presence or absence of hepatocellular carcinoma (i.e., their “HCC status”). In particular embodiments, the levels are in exosomes within the sample (i.e., an “exosome HCC biomarker”). In particular embodiments, the biomarkers are lipids, e.g., lipid classes or lipid species, or metabolites. The levels of each of the biomarkers need not be correlated with the HCC status in all subjects; rather, a correlation will exist at the population level, such that the level is sufficiently correlated within the overall population of individuals with HCC that it can be combined with the levels of other biomarkers, in any of a number of ways, as described elsewhere herein, and used to determine the HCC status. The values used for the measured level of the individual biomarkers can be determined in any of a number of ways, including direct readouts from relevant instruments or assay systems, e.g., using means known to those of skill in the art. In some embodiments, the readout values of the biomarkers are compared to the readout value of a reference or control, a lipid or other molecule whose level does not vary according to HCC status and whose level is measured at the same time as the biomarkers.

The term “correlating” generally refers to determining a relationship between one random variable with another. In various embodiments, correlating a given biomarker level with the presence or absence of HCC comprises determining the presence, absence or amount of at least one biomarker in a subject with the same outcome. In specific embodiments, a set of biomarker levels, absences or presences is correlated to a particular outcome, using receiver operating characteristic (ROC) curves.

In some embodiments, the biomarkers comprise one or more of the lipid classes sphingosine (SPH), sulfatide (ST), or lysophosphatidylserine (LysoPS). Sphingosine (see, e.g., PubChem CID No. 5280335, the entire disclosure of which is herein incorporated by reference) refers to a sphing-4-enine in which the double bond is trans, as well as variants thereof. Sulfatide (e.g., 3-O-sulfogalactosylceramide, or SM4, or sulfated galactocerebroside) refers to a class of sulfoglycolipids (see, e.g., ChemSpider ID 4573177, the entire disclosure of which is herein incorporated by reference). Lysophosphatidylserine (e.g., 1-stearoyl-sn-glycero-3-phosphoserine) is a 1-acyl-sn-glycerophosphoserine in which the acyl group is specified as stearoyl (see, e.g., PubChem CID No. 42607474, the entire disclosure of which is herein incorporated by reference). In some embodiments, the sphingosine class comprises the species SPH(t18:0). In some embodiments, the sulfatide class comprises the species ST(d18:1/20:2). IN some embodiments, the lysophosphatidylserine class comprises the species LysoPS(34:1). In some embodiments, the biomarkers comprise one or more lipid classes, lipid species, or metabolites listed in Table 1.

TABLE 1
Lipid classes, species, and metabolites enriched or reduced in exosomes
from subjects with HCC as compared to exosomes from healthy subjects.

| | Significant species |
|---|---|
| Lipid class enriched in HCC exosomes | |
| DLCL | DLCL(16:0/20:3) |
| Cardiolipin (CL) | CL(18:2/16:0/16:0/24:1), |
| | CL(18:2/18:0/18:0/24:1), |
| | CL(18:2/16:0/20:4/24:1) |
| Sphingosine (SPH) | SPH(t18:0) |
| (O-acyl)-1-hydroxy fatty acid (OAHFA) | OAHFA(18:2/32:0) |
| Lysophosphatidylserine (LysoPS) | LysoPS(34:1) |
| Phosphatidylglycerol (PG) | PG(18:0/18:2), PG(16:0/18:2) |
| Ceramide phosphoethanolamine (CerPE) | CerPE(d18:1/16:0) |
| Ceramide phosphate (CerP) | CerP(m17:0/22:6) |
| Dihexosylceramide (Hex2Cer) | Hex2Cer(d15:0/18:2), Hex2Cer(d14:0/20:4) |
| Hexosylceramide (Hex1Cer) | Hex1Cer(t20:0:18:2), Hex1Cer(d18:1/22:0) |
| Lipid class depleted in HCC exosomes | |
| Ganglioside (GD1a) | GD1a(d18:1/18:0), GD1a(d18:1/16:0) |
| Fatty acid (FA) | FA(20:4) |
| ST | ST(d18:1/20:2) |
| AcHexSiE | AcHexSiE(16:0) |

Other lipid species enriched in HCC exosomes

| | |
|---|---|
| PC(18:3e/22:4) | PC(16:1e/22:6) |
| SM(d14:0/23:1) | CerG3GNAcl(t18:0/24:1) |
| WE(26:5/18:0) | SPH(t18:0) |
| GM3(d18:1/22:0) | TG(25:0/16:0/17:0) |
| MGDG(16:0/21:6) | TG(18:0/14:0/16:0) |
| DG(20:0/16:0) | |

Other lipid species depleted in HCC exosomes

| | |
|---|---|
| PC(20:2e/18:1) | PC(18:1/24:2) |
| PE(16:0/20:4) | LPA(10:0) |
| Hex1Cer(d16:0/26:2) | PE(20:0p/20:3) |
| TG(18:1/10:3/18:3) | ST(d18:1/20:2) |
| PE(20:0p/18:1) | SM(t18:1/24:3) |

Metabolites enriched in HCC exosomes

| | |
|---|---|
| taurodeoxycholic acid (TDCA) | cholesterol sulfate |
| taurocholic acid (TCA) | LysoPC(18:2) |
| glycocholic acid (GCA) | |

Other biomarkers can also be used, e.g., in place of or in addition to any one or more of sphingosine, SPH(t18:0), sulfatide, ST(d18:1/20:2), lysophosphatidylserine, LysoPS(34:1), or biomarkers listed in Table 1. For example, in some embodiments, biomarkers used in the methods include one or more of PC(18:1/24:2), PE(20:0p/20:3), or LysoPC(18:2). In other embodiments, the biomarkers include, but are not limited to, any one or more of the lipid or metabolite biomarkers listed in any one or more of Tables 2, 3, 4, and/or 5. In some embodiments, the biomarkers include any one or more biomarkers, pairs of biomarkers, or sets of three biomarkers listed in Tables 7A, 7B, and/or 7C, respectively. It will be appreciated that any one or more of the biomarkers, or sets of biomarkers, listed in any one or more of Tables 1, 2, 3, 4, 5, 7A, 7B, or 7C, can be used alone or in any combination. Any number of biomarkers can be assessed in the methods, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100 or more biomarkers. In some embodiments, the selected biomarkers or combinations of biomarkers have an AUC score of at least about 0.6, 0.65, 0.7, 0.75, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or more with respect to their ability to distinguish between exosomes from HCC vs non-HCC samples. In some embodiments, any of the herein-disclosed biomarkers are detected along with alpha-fetoprotein (AFP), and the combined AUC score for the biomarker+AFP is determined. For example, in some embodiments, the biomarkers comprise any of the combinations of biomarkers (including AFP) listed in Table 4.

The biomarkers used in the present methods correspond to molecules whose levels in exosomes within biological samples, e.g., blood samples, particularly plasma samples, from the subject correlate with the presence of hepatocellular carcinoma (HCC). The level of the individual biomarkers can be elevated or depressed in individuals with HCC relative to the level in individuals without HCC. What is important is that the level of the biomarker is positively or inversely correlated with HCC, allowing the determination of a diagnosis in a subject based on a measurement of the biomarker level in a sample from the subject as described herein.

In some embodiments, AUC values are used as a measure of the ability of a biomarker or combination of biomarkers to determine the HCC infection status of an individual. The “area under curve” or “AUC” refers to area under a ROC curve. AUC under a ROC curve is a measure of accuracy. An area of 1 represents a perfect test, whereas an area of 0.5 represents an insignificant test. For suitable biomarkers as described herein, the AUC may be between 0.700 and 1. For example, the AUC may be at least about 0.700, at least about 0.750, at least about 0.800, at least about 0.810, at least about 0.820, at least about 0.830, at least about 0.840, at least about 0.850, at least about 0.860, at least about 0.870, at least about 0.880, at least about 0.890, at least about 0.900, at least about 0.910, at least about 0.920, at least about 0.930, at least about 0.940, at least about 0.950, at least about 0.960, at least about 0.970, at least about 0.980, at least about 0.990, or at least about 0.995.

Additional exosomal HCC biomarkers can be assessed and identified using any standard analysis method or metric, e.g., by analyzing data from exosome samples taken from subjects with or without a diagnosis of HCC, as described in more detail elsewhere herein and as illustrated, e.g., in the Examples. For example, in some embodiments, differences in AUC data between groups (e.g., between samples from HCC patients and samples from healthy patients without HCC) are evaluated using the Mann-Whitney U test. In some embodiments, principal component analysis (PCA) was performed with, e.g., the Euclidian-based distances matrix. Receiver operating characteristic (ROC) curves are generated, e.g., using the pROC package in R, and the AUCs calculated with a 95% confidence interval as well as sensitivity and specificity values. Binomial logistic regression analysis can be performed, e.g., for the analysis of combinations of multiple variables.

### III. Detecting Biomarker Levels

The levels of the biomarkers in the sample can be assessed in any of a number of ways. In particular embodiments, the sample, e.g., plasma sample, is fractionated to allow isolation of the exosomes, and the biomarker levels are determined in the fraction comprising the exosomes. The biomarker levels can be detected in any of a number of ways, including, but not limited to, gas chromatography, mass spectroscopy, gas chromatography-mass spectrometry, or liquid chromatograph-mass spectrometry. In particular embodiments, the levels of the biomarkers are determined using ultra-high resolution mass spectrometry. In some embodiments, an internal control is used, e.g., a reference molecule, e.g., lipid, whose level is known to not vary in correlation with the presence or absence of HCC. In some embodiments, one or more known biomarkers for HCC is assessed together with the herein-described biomarkers, e.g., alpha-fetoprotein or osteopontin.

In particular embodiments, the lipids in the exosome sample are profiled, e.g., by suspending exosomes in a standard solution such as Avanti SPLASH LIPIDOMIX mass spec standard, comprising deuterium labeled lipids. The lipids are prepared for mass spectrometry using standard methods, e.g., by precipitating proteins from the mixture, drying the samples, and resuspending in, e.g., ethanol. The resuspended sample can be used for the analysis, e.g., using a Thermo Fisher Scientific Accucore C30 column. Mass spectrometry can be performed, e.g., using a Thermo Fisher Scientific Orbitrap Fusion Lumos Tribrid mass spectrometer, and data analyzed, e.g., using software such as Thermo Scientific LipidSearch software.

In particular embodiments, metabolic profiling is performed, e.g., by adding an internal standard such as ¹³C₅-glutamic acid to pellets resuspended in, e.g., methanol. After centrifugation, e.g., at 17,000 g for 10 minutes, the supernatants are dried, and then reconstituted in solvent and prepared for analysis, e.g., liquid chromatography mass spectrometry (LC-MS) acquisition. In some embodiments, LC-MS is performed using, e.g., a Thermo Fisher Scientific Orbitrap Fusion mass spectrometer, and metabolites analyzed using software such as TraceFinder software and/or Compound Discoverer sofrware.

In some embodiments, the detection is carried out in whole or in part using an integrated system, as described elsewhere herein, which may also comprise a computer system as described elsewhere herein.

In some embodiments, replicates (e.g., triplicates) of any of the herein-described assays may be run for each sample in order to gain a higher level of confidence in the data.

Replicate values can be averaged, and standard deviations can be calculated.

In some embodiments, the herein-described methods for detecting biomarker levels are performed multiple times for an individual subject. For example, in some embodiments, the subject is undergoing treatment for HCC (e.g., a drug treatment, radiation treatment, and/or surgical treatment), and the samples are obtained at different time points during the treatment to assess the efficacy of the treatment. In some embodiments, the subject is known to be or believed to be at risk for HCC, and the samples are obtained at different time points to detect a potential evolution in the risk for HCC and/or to detect HCC as early as possible.

### IV. Determining HCC Status

To determine the presence of HCC (i.e., the “HCC status”) in an individual (i.e., a subject or patient), the measured biomarker levels in a sample obtained from the individual are generally compared to reference levels, e.g., levels taken from a healthy individual without HCC. The reference control levels can be measured at the same time as the biomarker levels, i.e., using the same sample, or can be a level determined based on previous measurements.

Thus, in one aspect, provided herein is a method of diagnosing HCC in a subject comprising, consisting essentially of, or consisting of: detecting differential levels of one or more hepatocellular carcinoma biomarkers in exosomes isolated from a blood (e.g., plasma) sample from the subject as compared to a control, wherein the one or more hepatocellular carcinoma biomarkers comprise one or more of the lipid classes sphingosine (SPH), sulfatide (ST), and/or lysophosphatidylserine (LysoPS), and/or any one or more of the lipid classes, lipid species, metabolites, or combinations thereof, listed in any one or more of Table 1, Table 2, Table 3, Table 4, Table 5, Table 7A, Table 7B, or Table 7C.

In some embodiments, the method comprises: providing exosomes isolated from a blood (e.g., plasma) sample from the subject; detecting the one or more hepatocellular carcinoma biomarkers in the exosomes, and comparing the levels of the one or more hepatocellular carcinoma biomarkers to a control, wherein the level of SPH is elevated, the level of ST is reduced, and/or the level of LysoPS is elevated in the exosomes relative to control levels determined from exosomes of a healthy individual without hepatocellular carcinoma.

When using multiple biomarkers, it is not necessary that all of the biomarkers are elevated or depressed relative to control levels in a sample, e.g., an exosome-comprising sample, from a given subject to give rise to a determination of HCC. For example, for a given biomarker level there can be some overlap between individuals falling into different probability categories. However, collectively the combined levels for all of the biomarkers included in the assay gives rise to an AUC score that indicates a high probability of, e.g., the presence of HCC.

In some embodiments, the levels of the selected biomarkers are quantified and compared to one or more preselected or threshold levels. Threshold values can be selected that provide an ability to predict the presence or absence of HCC. Such threshold values can be established, e.g., by calculating receiver operating characteristic (ROC) curves using a first population with HCC and a second population without HCC.

The present disclosure provides methods of generating a classifier(s) (also referred to as training) for use in the methods of determining the presence or absence of HCC in a subject. As used herein, the terms “classifier” and “predictor” are used interchangeably and refer to a mathematical function that uses the values of the signature (e.g., lipid or metabolite levels from a defined set of biomarkers) and a pre-determined coefficient for each signature component to generate scores for a given observation or individual patient for the purpose of assignment to a category. A classifier is linear if scores are a function of summed signature values weighted by a set of coefficients. Furthermore, a classifier is probabilistic if the function of signature values generates a probability, a value between 0 and 1.0 (or 0 and 100%) quantifying the likelihood that a subject or observation belongs to a particular category or will have a particular outcome, respectively. Probit regression and logistic regression are examples of probabilistic linear classifiers.

A classifier, including a linear classifier, may be obtained by a procedure known as training, which consists of using a set of data containing observations with known category membership (e.g., subjects with HCC or without HCC). Specifically, training seeks to find the optimal coefficient for each component of a given signature, where the optimal result is determined by the highest classification accuracy. In some embodiments, a unique classifier may be developed and trained with respect to a particular platform upon which the signature is measured.

For example, classifiers that use host lipid or metabolite biomarker levels can be generated from a training set of samples obtained from patients having a known HCC status. Measurements of many host lipids and metabolites can be obtained, e.g., as disclosed elsewhere herein. The measurements can be analyzed to determine sets of biomarkers (i.e., their levels) that best discriminate between the different classifications of the training set via an optimization procedure. The analysis of lipid or metabolite level data can include training a machine learning model to distinguish between positive and negative samples based on the levels of certain lipid or metabolite biomarkers. The analysis can include using the data as a training set where the biomarker levels and known diagnosis are used to train a machine learning model to distinguish between positive and negative samples. In the process of learning, the model identifies lipid and/or metabolite biomarkers that are predictive for HCC.

Hence, one aspect of the present disclosure provides a method of making an HCC classifier comprising, consisting of, or consisting essentially of (i) obtaining a biological sample such as a blood or plasma sample from a plurality of subjects suffering from HCC; (ii) isolating exosomes from the samples and processing the lipid or metabolite fraction from the sample; (iii) measuring the levels of a plurality of lipids or metabolites; normalizing the levels; generating as HCC classifier to include normalized biomarker levels and a “weighting” coefficient value; and optionally, (vi) uploading the classifier (e.g., lipid/metabolite identity and weighing coefficient) to a database.

In some embodiments, the method further includes uploading the final lipid/metabolite target list for the generated classifier, the associated weights (wₙ), and threshold values to one or more databases.

In some embodiments, the measuring comprises the detection and quantification (e.g., semi-quantification) of the selected biomarkers in the sample. In some embodiments, the measured biomarker levels are adjusted relative to one or more standard level(s) (“normalized”). As known in the art, normalizing is done to remove technical variability inherent to a platform to give a quantity or relative quantity (e.g., of lipid classes or species, or metabolites).

In some embodiments, the measurement of differential levels of specific biomarkers from biological samples may be accomplished using a range of technologies, reagents, and methods. These include any of the methods of measurement as described elsewhere herein.

The biomarker levels are typically normalized following detection and quantification as appropriate for the particular platform using methods routinely practiced by those of ordinary skill in the art.

In some embodiments, the signatures may be obtained using a supervised statistical approach known as sparse linear classification in which sets of gene products are identified by the model according to their ability to separate phenotypes during a training process that uses the selected set of patient samples. The outcomes of training is a biomarker signature(s) and classification coefficients for the classification comparison. Together the signature(s) and coefficient(s) provide a classifier or predictor. Training may also be used to establish threshold or cut-off values.

Threshold or cut-off values can be adjusted to change test performance, e.g., test sensitivity and specificity. For example, the threshold for HCC may be intentionally lowered to increase the sensitivity of the test for HCC, if desired.

In some embodiments, the classifier generating comprises iteratively: (i) assigning a weight for each normalized biomarker level value, entering the weight and expression value for each biomarker into a classifier (e.g., a linear regression classifier) equation and determining a score for outcome for each of the plurality of subjects, then (ii) determining the accuracy of classification for each outcome across the plurality of subjects, and then (iii) adjusting the weight until accuracy of classification is optimized. Biomarkers having a non-zero weight are included in the respective classifier.

Determining the accuracy of classification may involve the use of accuracy measures such as sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting HCC.

In some embodiments, the classifier is a linear regression classifier and said generating comprises converting a score of said classifier to a probability using a link function. As known in the art, the link function specifies the link between the target/output of the model (e.g., probability of HCC) and systematic components (in this instance, the combination of explanatory variables that comprise the predictor) of the linear model. It says how the expected value of the response relates to the linear predictor of explanatory variable.

In some embodiments, the classifiers that are developed during training and using a training set of samples are applied for prediction purposes to diagnose new individuals (“classification”). For each subject or patient, a biological sample is taken and the normalized biomarker levels (i.e., the relative amounts of biomarkers) in the sample of each of the biomarkers specified by the signatures found during training are the input for the classifier. In other embodiments, the classifier can also use the weighting coefficients discovered during training for each gene product. As outputs, the classifiers are used to compute probability values. Each probability value may be used to determine the presence or absence of HCC in the subject.

In some embodiments, these values may be reported relative to a reference range that indicates the confidence with which the classification is made. In some embodiments, the output of the classifier may be compared to a threshold value, for example, to report a “positive” in the case that the classifier score or probability exceeds the threshold indicating the presence of HCC. If the classifier score or probability fails to reach the threshold, the result would be reported as “negative” for the respective condition.

It should be noted that a classifier obtained with one platform may not show optimal performance on another platform. This could be due to the promiscuity of probes or other technical issues particular to the platform. Accordingly, also described herein are methods to adapt a signature as taught herein from one platform for another.

It will be appreciated that for any particular biomarker, a distribution of biomarker levels for subjects with and without HCC may overlap. Under such conditions, a test does not absolutely distinguish the two populations (i.e., with or without HCC) with 100% accuracy, and the area of overlap indicates where the test cannot distinguish them. A threshold value is selected, above which the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982)).

In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more biomarkers are selected to discriminate between subjects with HCC and subjects without HCC with at least about 70%, 75%, 80%, 85%, 90%, 95% accuracy or having a C-statistic of at least about 0.70, 0.75, 0.80, 0.85, 0.90, 0.95.

The phrases “assessing the likelihood” and “determining the likelihood,” as used herein, refer to methods by which the skilled artisan can predict the presence or absence of a condition (e.g., HCC) in a patient. The skilled artisan will understand that this phrase includes within its scope an increased probability that a condition (e.g., HCC) is present or absent in a patient; that is, that a condition is more likely to be present or absent in a subject. For example, the probability that an individual identified as having a specified condition actually has the condition can be expressed as a “positive predictive value” or “PPV.” Positive predictive value can be calculated as the number of true positives divided by the sum of the true positives and false positives. PPV is determined by the characteristics of the predictive methods of the present methods as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition or outcome actually does not have that condition can be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined to have a significant probability of having or not having a specified condition or outcome (e.g., HCC). By “significant probability” is meant that the subject has a reasonable probability (0.6, 0.7, 0.8, 0.9 or more) of having, or not having, a specified condition or outcome.

In some embodiments, a detection of HCC can be based not solely on biomarker levels, but can also take into account clinical and/or other data about the subject, e.g., clinical data about the subject's current medical state (e.g., the presence of cirrhosis or fibrosis, and the state of advancement of such cirrhosis or fibrosis), the presence of any symptoms characteristic of HCC, the medical history of the subject, the presence of one or more risk factors for HCC, and/or demographic data about the subject (age, sex, etc.).

### V. Treatment Decisions

The detection of HCC in a subject using the present methods and compositions can indicate the delivery of medical care appropriate for, e.g., the stage, form, or other properties of the detected HCC. In some embodiments, the subject receives treatment such as a drug treatment, radiation treatment, and/or surgical treatment.

Thus, in one aspect, provided herein is a method for treating HCC in a subject comprising, consisting essentially of, or consisting of: administering an effective amount of an anti-hepatocellular carcinoma treatment to a subject having differential levels of one or more hepatocellular carcinoma biomarkers in exosomes isolated from a blood sample from the subject as compared to a control, wherein the one or more hepatocellular carcinoma biomarkers comprise one or more of the lipid classes sphingosine (SPH), sulfatide (ST), and/or lysophosphatidylserine (LysoPS), and/or any one or more of the lipid classes, lipid species, metabolites, or combinations thereof, listed in any one or more of Table 1, Table 2, Table 3, Table 4, Table 5, Table 7A, Table 7B, or Table 7C.

In some embodiments, the method comprises: providing exosomes isolated from a blood sample from the subject; detecting the one or more hepatocellular carcinoma biomarkers in the exosomes, and comparing the levels of the one or more hepatocellular carcinoma biomarkers to a control, wherein the level of SPH is elevated, the level of ST is reduced, and/or the level of LysoPS is elevated in the exosomes relative to control levels determined from exosomes of a healthy individual without hepatocellular carcinoma.

In some embodiments, a patient with HCC as detected using the present methods receives surgical treatment for the HCC. For example, the patient may receive surgical resection (removal of the tumor with surgery) or a liver transplant. Small liver cancers may also be treated with other types of treatment such as ablation or radiation.

In some embodiments, a patient with HCC as detected using the present methods receives partial hepatectomy. Partial hepatectomy is surgery to remove part of the liver. Only people with good liver function who are healthy enough for surgery and who have a single tumor that has not grown into blood vessels can have this operation. In such cases, imaging tests such as CT or MRI with angiography are done first to see if the cancer can be removed completely.

Most patients with liver cancer in the United States also have cirrhosis. In someone with severe cirrhosis, removing even a small amount of liver tissue at the edges of a cancer might not leave enough liver behind to perform important functions. People with cirrhosis are typically eligible for surgery if there is only one tumor (that has not grown into blood vessels) and they will still have a reasonable amount (at least 30%) of liver function left once the tumor is removed. Doctors often assess this function by assigning a Child-Pugh score, which is a measure of cirrhosis based on certain lab tests and symptoms. Patients in Child-Pugh class A are most likely to have enough liver function to have surgery. Patients in class B are less likely to be able to have surgery. Surgery is not typically an option for patients in class C.

In some embodiments, a patient with HCC as detected using the present methods receives a liver transplant. Liver transplants can be an option for those with tumors that cannot be removed with surgery, either because of the location of the tumors or because the liver has too much disease for the patient to tolerate removing part of it. In general, a transplant is used to treat patients with small tumors (either 1 tumor smaller than 5 cm across or 2 to 3 tumors no larger than 3 cm) that have not grown into nearby blood vessels. It can also rarely be an option for patients with resectable cancers. With a transplant, not only is the risk of a second new liver cancer greatly reduced, but the new liver will function normally.

In some embodiments, a patient with HCC as detected using the present methods is treated using ablation. Ablation is treatment that destroys liver tumors without removing them. These techniques can be used in patients with a few small tumors and when surgery is not a good option. They are less likely to cure the cancer than surgery, but they can still be very helpful for some people. These treatments are also sometimes used in patients waiting for a liver transplant. Ablation is best used for tumors no larger than 3 cm across. For slightly larger tumors (1 to 2 inches, or 3 to 5 cm across), it may be used along with embolization. Because ablation often destroys some of the normal tissue around the tumor, it might not be a good choice for treating tumors near major blood vessels, the diaphragm, or major bile ducts. In some embodiments, the ablation is radiofrequency ablation (RFA). In some embodiments, the ablation is microwave ablation (MWA). In some embodiments, the ablation is cryoablation (cryotherapy). In some embodiments, the ablation is ethanol (alcohol) ablation, e.g., percutaneous ethanol injection (PEI).

In some embodiments, a patient with HCC as detected using the present methods is treated using embolization therapy. Embolization is a procedure that injects substances directly into an artery in the liver to block or reduce the blood flow to a tumor in the liver. The liver has two blood supplies. Most normal liver cells are fed by the portal vein, whereas a cancer in the liver is mainly fed by the hepatic artery. Blocking the part of the hepatic artery that feeds the tumor helps kill off the cancer cells, but it leaves most of the healthy liver cells unharmed because they get their blood supply from the portal vein. Embolization is an option for some patients with tumors that cannot be removed by surgery. It can be used for people with tumors that are too large to be treated with ablation (usually larger than 5 cm across) and who also have adequate liver function. It can also be used with ablation. Embolization can reduce some of the blood supply to the normal liver tissue, so it may not be a good option for some patients whose liver has been damaged by diseases such as hepatitis or cirrhosis. In some embodiments, the embolization is trans-arterial embolization (TAE). In some embodiments, the embolization is trans-arterial chemoembolization (TACE). In some embodiments, the embolization is drug-eluting bead chemoembolization (DEB-TACE). In some embodiments, the embolization is radioembolization (RE).

In some embodiments, a patient with HCC as detected using the present methods is treated using radiation therapy. Radiation therapy uses high-energy rays, or particles to destroy cancer cells. This option may not be advised for the patient whose liver has been greatly damaged by disease such as hepatitis or cirrhosis. Radiation can be helpful in treating: liver cancer that cannot be removed by surgery; liver cancer that cannot be treated with ablation or embolization or did not respond well to those treatments; liver cancer that has spread to other areas such as the brain or bones; patients experiencing severe pain due to large liver cancers; patients having a tumor thrombus blocking the portal vein.

In some embodiments, a patient with HCC as detected using the present methods is treated using drug therapy, e.g., targeted drug therapy, immunotherapy, or chemotherapy. Targeted drugs work differently from standard chemotherapy drugs and include, e.g., kinase inhibitors; Sorafenib (Nexavar), lenvatinib (Lenvima), Regorafenib (Stivarga), and cabozantinib (Cabometyx). Immunotherapy can comprise the administration of monoclonal antibodies. Monoclonal antibodies are designed to attach to a specific target. The monoclonal antibodies used to treat liver cancer affect a tumor's ability to form new blood vessels, also known as angiogenesis. These therapeutics are often referred to angiogenesis inhibitors and include: Bevacizumab (Avastin), which can be used in conjunction with the immunotherapy drug atezolizumab (Tecentriq); Ramucirumab (Cyramza).

An important part of the immune system is its ability to keep itself from attacking normal cells in the body. To do this, it uses “checkpoints”—proteins on immune cells that need to be switched on or off to start an immune response. Cancer cells sometimes use these checkpoints to avoid being attacked by the immune system. Newer drugs that target these checkpoints hold a lot of promise as liver cancer treatments and include: PD-1 and PD-L1 inhibitors; Atezolizumab (Tecentriq), which can be used in conjunction with the targeted drug bevacizumab (Avastin); Pembrolizumab (Keytruda) and nivolumab (Opdivo), alone or in combination with ipilimumab (described below) may also be an option.

Ipilimumab (Yervoy® blocks CTLA-4, another protein on T cells that normally helps keep them in check. This drug can be used in combination with nivolumab to treat liver cancer that has previously been treated, such as with the targeted drug sorafenib [Nexavar®]. The combination of the two drugs may help shrink the cancer more than nivolumab alone.

The most common chemotherapy drugs for treating liver cancer include: Gemcitabine (Gemzar); Oxaliplatin (Eloxatin); Cisplatin; Doxorubicin (pegylated liposomal doxorubicin); 5-fluorouracil (5-FU); Capecitabine (Xeloda); Mitoxantrone (Novantrone), or combinations thereof. Chemotherapy can be regional when drugs are inserted into an artery that leads to the part of the body with the tumor. thereby focusing the chemo on the cancer cells in that area and reducing side effects by limiting the amount of drug reaching the rest of the body. Hepatic artery infusion (HAI), or chemo given directly into the hepatic artery, is an example of a regional chemotherapy that can be used for liver cancer. It is slightly different from chemoembolization because surgery is needed to put an infusion pump under the skin of the abdomen. The pump is attached to a catheter that connects to the hepatic artery. This is done while the patient is under general anesthesia. The chemo is injected with a needle through the skin into the pump' reservoir and it is released slowly and steadily into the hepatic artery. The drugs most commonly used for HAI include floxuridine (FUDR), cisplatin, and oxaliplatin.

### VI. Kits and Systems

### A. Kits

In one aspect, kits are provided for the detection of HCC in a subject, wherein the kits can be used to detect the biomarkers described herein. The kit may include, e.g., one or more agents for the detection of biomarkers, a container for holding a biological sample, e.g., plasma sample, isolated from a human subject suspected of having HCC; and instructions for reacting agents with the biological sample or a portion of the biological sample to detect the presence or amount of at least one biomarker in the biological sample. The agents may be packaged in separate containers. The kit may further comprise one or more control reference samples and reagents for performing the herein-described methods. The kit may also comprise one or more devices or implements for carrying out any of the herein methods.

In certain embodiments, the kit comprises agents for measuring the levels of one or more sphingosine (SPH), sulfatide (ST), or lysophosphatidylserine (LysoPS), such as SPH(t18:0), ST(d18:1/20:2), or LysoPS(34:1). In some embodiments, the kit comprises agents for measuring the levels of one or more of PC(18:1/24:2), PE(20:0p/20:3), or LysoPC(18:2). In some embodiments, the kit comprises agents for measuring the levels of any one or more biomarkers or sets of biomarkers listed in any one or more of Table 1, Table 2, Table 3, Table 4, Table 5, Table 7A, Table 7B, or Table 7C, or any combination of any of the biomarkers or sets of biomarkers listed in any one or more of Table 1, Table 2, Table 3, Table 4, Table 5, Table 7A, Table 7B, or Table 7C.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing instructions for methods of diagnosing HCC.

### B. Measurement Systems and Reports for Detecting and Recording Biomarker Expression

In one aspect, a system, e.g., measurement system is provided. Such systems allow, e.g., the detection of biomarker levels in a sample and the recording of the data resulting from the detection. The stored data can then be analyzed to determine the HCC status of a subject. Such systems can comprise, e.g., assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

Also provided is a system for detecting hepatocellular carcinoma biomarkers in a sample, by utilizing a station for analyzing the sample by mass spectrometry (Mass Spec or MS) or liquid chromatography/mass spectrometry (LC/MS) to detect two or more HCC biomarkers in the sample, wherein the two or more HCC biomarkers are two or more of sphingosine (SPH), sulfatide (ST), lysophosphatidylserine (LysoPS), SPH(t18:0), ST(d18:1/20:2), LysoPS(34:1), PC(18:1/24:2), PE(20:0p/20:3), LysoPC(18:2), or any of the biomarkers or sets of biomarkers listed in any one or more of Table 1, Table 2, Table 3, Table 4, Table 5, Table 7A, Table 7B, or Table 7C; the sample is a blood sample, e.g., plasma sample, obtained from a subject, and the report is useful for diagnosing hepatocellular carcinoma in the subject. Optionally, a station for generating a report containing information on results of the analyzing is further included.

Also provided is a method of generating a report containing information on results of the detection of HCC biomarkers in a sample, including detecting two or more hepatocellular carcinoma biomarkers in the sample, and generating the report, wherein the two or more HCC biomarkers are two or more of sphingosine (SPH), sulfatide (ST), lysophosphatidylserine (LysoPS), SPH(t18:0), ST(d18:1/20:2), LysoPS(34:1), PC(18:1/24:2), PE(20:0p/20:3), LysoPC(18:2), or any of the biomarkers listed in any one or more of Table 1, Table 2, Table 3, Table 4, Table 5, Table 7A, Table 7B, or Table 7C; the sample is a blood sample, e.g., plasma sample, obtained from a subject, and the report is useful for diagnosing hepatocellular carcinoma in the subject.

### C. Computer Diagnostic Systems for Determining HCC Status

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that uses inputted biomarker expression (and optionally other) data, and determines the HCC status of a subject.

A computer system can include desktop and laptop computers, tablets, mobile phones and other mobile devices. The system can include various elements such as a printer, keyboard, storage device(s), monitor (e.g., a display screen, such as an LED), peripherals, devices to connect a computer system to a wide area network such as the Internet, a mouse input device, scanner, a storage device(s), computer readable medium, camera, microphone, accelerometer, and the like. Any of the data mentioned herein can be output from one component to another component and can be output to the user.

In one aspect, the present disclosure provides a computer implemented method for determining the presence or absence of HCC in a patient. The computer performs steps comprising, e.g., receiving inputted patient data comprising values for the levels of one or more biomarkers in a biological sample from the patient; analyzing the levels of one or more biomarkers and optionally comparing them to respective reference values, optionally comparing the biomarker levels to one or more threshold values to determine HCC status; and displaying information regarding the HCC status or probability in the patient. In certain embodiments, the inputted patient data comprises values for the levels of a plurality of biomarkers in a biological sample from the patient, e.g., biomarkers comprising one or more pairs or three-way combinations of biomarkers listed in one or more of Tables 7A, 7B, or 7C, and/or for any combination comprising two or more biomarkers from the following list: sphingosine (SPH), sulfatide (ST), lysophosphatidylserine (LysoPS), SPH(t18:0), ST(d18:1/20:2), LysoPS(34:1), PC(18:1/24:2), PE(20:Op/20:3), and LysoPC(18:2).

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the HCC status of the subject. For example, the storage component includes instructions for determining HCC status based on biomarker levels, as described herein. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### VII. Examples

The following examples are offered to illustrate, but not to limit, the claimed invention. Additional examples and figures can be found in Sanchez et al., Lipidomic Profiles of Plasma Exosomes Identify Candidate Biomarkers for Early Detection of Hepatocellular Carcinoma in Patients with Cirrhosis, Cancer Prev. Res. 14:955-62 (2021), which is incorporated herein in its entirety for all purposes.

### Example 1. Lipidomic and Metabolomic Profiles of Plasma Exosomes Identify Biomarkers for Early HCC Detection in Patients with Cirrhosis

Novel biomarkers for surveillance of HCC in cirrhotic patients are urgently needed. Exosomes and their lipid and metabolite content in particular, represent potentially valuable noninvasive diagnostic biomarkers. We isolated exosomes from plasma of 72 cirrhotic patients, including 31 with HCC. Exosomes and unfractionated plasma samples were processed for nontargeted lipidomics and metabolomics using ultra-high resolution mass spectrometry. A total of 2,864 lipid species, belonging to 52 lipid classes, and 93 metabolites were identified. Exosome fractionation and HCC diagnosis had significant impact on the lipid and metabolite profiles. Ten lipid classes and 5 metabolites were enriched while 4 lipid classes were depleted, in HCC exosomes compared to non-HCC exosomes. These HCC-associated changes reflected alterations in glycerophospholipid metabolism, arachidonic acid metabolism, ferroptosis and primary bile acid biosynthesis. With AUCs ranging from 0.86 to 0.94, exosomal sphingosine (SPH), sulfatide (ST), and lysophosphatidylserine (LysoPS) had significantly higher performance in HCC diagnosis than alpha-fetoprotein (AFP) (AUC=0.80). Their performance further increased when combined with AFP (AUCs=0.93-0.96). Selected individual analytes such as PC(18:1/24:2), PE(20:0p/20:3) and LysoPC(18:2) also had high performance in HCC diagnosis when combined with AFP (AUCs=0.97, 0.96 and 0.89, respectively). The combination of AFP+SPH and of AFP+ST reached 90% sensitivity at 97% specificity and 95% sensitivity at 90% specificity, respectively, for detection of early stage HCC compared to 45% sensitivity at 95% specificity for AFP at 20 ng/ml. In conclusion, this study identified promising biomarkers for early detection of HCC as well as pathways altered in HCC exosomes that may contribute to tumor development and progression.

### A. Methods

### 1. Study Participants

This study includes 72 participants with cirrhosis (31 with HCC and 41 without HCC), matched by gender, age and etiology (Table 6). Participants were recruited from Hepatology and multidisciplinary HCC clinics at Parkland Memorial Health and Hospital System and UT Southwestern Medical Center, using protocols previously described in detail (24, 25). In brief, cirrhosis was diagnosed histologically, radiographically, or using non-invasive markers of fibrosis (26). All HCC diagnoses were confirmed using the American Association for the Study of Liver Disease criteria and staging performed using Barcelona Clinic Liver Cancer (BCLC) staging system (27). All HCC cases were treatment-naïve at time of recruitment, with blood samples processed and stored within 4 hours of collection. The same blood draws were used for both clinical lab measurements as part of routine clinical care (such as AFP values used in the study) and exosomes analysis. Heavy alcohol was defined as more than 1 or 2 drinks per day for women and men, respectively. Ascites and hepatic encephalopathy were classified as none, mild or controlled, and severe or uncontrolled. Mild or controlled ascites was defined as small ascites on imaging or adequately treated with diuretics. Mild or controlled hepatic encephalopathy was defined as adequately treated on lactulose and/or rifaximin. Patients requiring admission or other interventions, such as paracentesis, were determined to have severe or uncontrolled hepatic decompensation. MELD and Child Pugh scores were calculated per readily available clinical calculators.

### 2. Exosome Isolation

Stored aliquots of 500 μL EDTA plasma were thawed on ice and subjected to serial centrifugation to remove cellular debris. Subsequently, 5 μL of plasma was snap frozen and stored at −80° C. The remainder plasma samples and a blank sample of phosphate-buffered saline (PBS) were ultracentrifuged as previously described (28). Briefly, samples were diluted with equal parts of PBS and centrifuged in an Optima MAX-XP bench top ultracentrifuge with TLA-55 rotor (Beckman Coulter) in polypropylene tubes (Cat #357448, Beckman Coulter Inc) at 150,000 g 4 degrees Celsius for 2 h. The pellets were carefully washed with PBS and centrifuged again at 150,000 g 4° C. for 2 h in polypropylene tubes. The resulting pellets were snap frozen and immediately stored at −80 degrees Celsius for metabolomics and lipidomics profiling at the Proteomics and Metabolomics Core at MD Anderson Cancer Center.

### 3. Lipidomic Profiling

Exosome pellets, unfractionated plasma and blank samples were subjected to Avanti SPLASH® LIPIDOMIX® Mass Spec Standard (330707) in methanol, 0.5 μL of 10 mM butylated hydroxytoluene in methanol, and 189.5 μL of −80 degrees Celsius ethanol and vortexed. The contents of the mixture were then transferred to a Phenomenex Impact Protein Precipitation Plate (CEO-7565) and filtered through using a vacuum manifold. The sample tubes were next rinsed with 200 μL of ethanol that was subsequently used to elute residual lipids from the protein precipitation plate. The sample was transferred to a glass autosampler vial and dried using a centrifugal vacuum concentrator. Dried samples were reconstituted in 50 μL ethanol. The injection volume was 10 μL. Mobile phase A (MPA; weak) was 40:60 acetonitrile:0.1% formic acid in 10 mM ammonium acetate. Mobile phase B (MPB; strong) was 90:8:2 isopropanol:acetonitrile:0.1% formic acid in 10 mM ammonium acetate. The chromatographic method included a Thermo Fisher Scientific Accucore C30 column (2.6 μm, 150×2.1 mm) maintained at 40 degrees Celsius, autosampler tray chilling at 8 degrees Celsius, a mobile phase flowrate of 0.200 mL/min, and a gradient elution program as follows: 0-7 min, 20-55% MPB; 7-8 min, 55-65% MPB; 8-12 min, 65% MPB; 12-30 min, 65-70% MPB; 30-31 min, 70-88% MPB; 31-51 min, 88-95% MPB; 51-53 min, 95-100% MPB; 53-60 min, 100% MPB; 60-60.1 min 100-20% MPB; 60.1-70 min, 20% MPB. A Thermo Fisher Scientific Orbitrap Fusion Lumos Tribrid mass spectrometer with heated electrospray ionization source was operated in data dependent acquisition mode, in both positive and negative ionization modes, with scan ranges of 150-677 and 675-1500 m/z. An Orbitrap resolution of 120,000 (FWHM) was used for MS1 acquisition and a spray voltages of 3,600 and −2900 V were used for positive and negative ionization modes, respectively. For MS2 and MS3 fragmentation a hybridized HCD/CID approach was used. Each sample was analyzed using in both ionization modes using four 10 μL injections making use of the two aforementioned scan ranges. Data were analyzed using Thermo Scientific LipidSearch software (version 4.2.23) and R scripts written in house. The peak areas (area-under-the-curve; AUC) identified in Thermo Scientific LipidSearch software were exported to Microsoft Excel.

### 4. Metabolomic Profiling

Exosome pellets, unfractionated plasma and blank samples were thawed on ice and 80 μL of methanol including 2 μM ¹³C₅-glutamic acid (internal standard) was added to each sample. Plasma samples were subjected to an additional 80 μL of methanol and 15 μL of water. Samples were then vortexed for 20 min and centrifuged at 17,000 g for 10 min. The resulting supernatants were then transferred into new tubes and dried under vacuum. Dried samples were reconstituted in 11 μl reconstitution solvent (20% deionized water, 30% methanol, 50% acetone nitrile), sonicated, vortexed, centrifuged, and transferred into vials for analysis. A total of 10 μl of each sample was injected for liquid chromatography mass spectrometry (LC-MS) acquisition. LC-MS analysis was performed on a Thermo Fisher Scientific Orbitrap Fusion mass spectrometer in data dependent acquisition mode, in both positive and negative ionization modes. LC separation of metabolites with hydrophilic interaction liquid chromatography (HILIC) was performed using an Xbridge BEH Amide column on a Vanquish LC (Thermo Fisher) and a multistep gradient using water as MPA, and acetonitrile in MPB, both containing 0.10% formic acid, with an elution gradient of 99% MPA at 0-2 min, 70-85% MPA at 3-21 min, and 99% MPA at 22-25 min. The gradient operated at a flow rate of 0.35 mL/min and was maintained at 45 degrees Celsius. A resolution of 120,000 using pos/neg polarity switching with a scan range of 80-800 m/z was used for MS¹ acquisition. For MS² acquisition, an ion trap mass analyzer was used with high-energy collision-induced dissociation, collision energy of 30 V.

A database was constructed of 435 compounds previously reported for the Xbridge BEH Amide column by Liu et al. (29) (341 compounds) and by Yuan et al. (30) (274 compounds). Only compounds that could be mapped to Human Metabolome Database (HMDB) entry (31) were withheld. Next, we used that database to manually curate peak quality peaks using TraceFinder (ver.5.0) software in both positive and negative mode, which resulted in a list of 80 targeted compounds (only proton loss or proton gain surveyed). Non-targeted analysis was conducted using Compound Discoverer (ver.3.1) software. Feature tables from the Compound Discoverer were exported to Excel and were additionally filtered using the following steps: 1) Only features with a compound name were selected, 2) A data-dependent MS² scan must have been acquired, 3) Features must have a predicted formula, 4) Only compounds that could be mapped to HMDB entry were included. This resulted in a database of 279 compounds used for manual curation to select high-quality peaks using TraceFinder, which resulted in a shortlist of 105 compounds. The results from the targeted and non-targeted searches were combined to construct a final database of 135 compounds (45 compounds were overlapping) which underwent a final review using TraceFiner, after which MS¹ peak areas were exported to Excel. Data-dependent MS² spectra were the basis for metabolite identification. Trifluoroacetic acid and phosphoric acid were removed. Compounds found in both TraceFinder and Compound Discoverer were merged into one final non-targeted list and high quality peaks were manually processed in TraceFinder to determine AUC, and the data were exported to Excel.

### 5. Statistical Analysis

Demographic and clinical parameters were compared between HCC and non-HCC patients using two tailed t-test for continuous variables and Fisher test for categorical variables. Lipidomic AUC data were normalized by total signal while metabolomics AUC data were normalized by the internal standard followed by total signal. AUC peak data were filtered using the blank sample as background and full analysis was performed on analytes identified in at least 20% of the samples. The difference in AUCs between HCC and non-HCC was evaluated using Mann-Whitney U test adjusted by Benjamini-Hochberg method (32) to reduce the likelihood of false positives. Principal component analysis (PCA) was performed with the Euclidian-based distances matrix, generated in R using log 10-transformed values. Pie graphs, volcano plots, scatter plots and spearman correlation analysis were generated in Graph Prism 8.0.0. The pROC package in R was used to generate receiver operating characteristic (ROC) curves, and compute AUC with 95% confidence interval (CI).

Sensitivity and specificity values were calculated using Youden index defined as the maximum of sensitivity+specificity −1 along a ROC curve. For analyses using a combination of multiple variables, binomial logistic regression analysis was first performed on the variables; the fitted probabilities were then used for ROC curve generation.

For 3-fold cross-validation, the caret package in R was used to randomly split subjects into three equal groups, creating a testing set and two training sets. The logistic model was fit using the training set and glm function and the fitted model was used for prediction of the testing set. This was done three times which resulted in three sets of predictions and three sets of labels. The list of predictions and labels were put into the cvAUC function which resulted in the AUC for each fold and the mean AUC. The list of lipid classes and lipid species found to be depleted or enriched in HCC exosomes vs non-HCC exosomes were analyzed using LIPEA while enriched metabolites, lipid class and lipid species were analyzed using metaboanalyst. The resulting pathways were combined. To determine the association between abundance of individual lipid classes or metabolites and HCC, Firth logistic regression (33) was performed using the brglm package in R, with and without adjusting for age, gender and BMI. For each lipid class and metabolite enriched in HCC exosomes, we estimated the odds ratio (OR) and adjusted OR (AOR) for HCC with high abundance (Tertile T3). For each lipid class depleted in HCC exosomes, and often undetected in HCC exosomes, we estimated the OR and AOR for HCC with the lipid class as absent versus detected.

### B. Results

1. Exosome Isolation from Plasma of Cirrhosis Patients with or without HCC

We collected plasma from 72 patients with cirrhosis, 31 with HCC (HCC) and 41 without HCC (non-HCC). Detailed demographic and clinical parameters of the study participants are provided in Table 6. Non-HCC patients were selected so that gender, age and etiology were not statistically different between HCC patients and non-HCC patients. The average age of HCC patients was 62.4 and 59 in non-HCC patients. Hepatitis C virus (HCV) was the most common etiology, representing 45% of HCC patients and 44% of non-HCC patients, followed by EtOH (23% in HCC patients and 24% in non-HCC patients). Child Pugh class A was the most common class, accounting for 58% in HCC patients and 68% in non-HCC patients. The majority of HCC patients (64%) had early stage disease, defined as BCLC stage 0 or A. As expected, patients with HCC had higher AFP levels than non-HCC patients (median 24 ng/mL versus 5 ng/mL, p<0.021).

Exosomes from the 72 plasma samples were isolated by ultracentrifugation, the gold standard method for exosome isolation. Unfractionated plasma samples and isolated exosomes were processed for metabolomics and lipidomics by mass spectrometry. No significant differences in amounts of total metabolites were detected between HCC and non-HCC in plasma samples (fold change (FC)=0.92, p=0.15), nor in exosomes (FC=0.95, p=0.78). While similarly no significant differences in total lipids were detected in HCC versus non-HCC plasma samples (FC=1.13, p=0.21), a decrease in total lipids was observed in HCC exosomes compared to non-HCC exosomes (FC=0.64, p=0.005).

### 2. Lipidomic Profiling of Exosomes

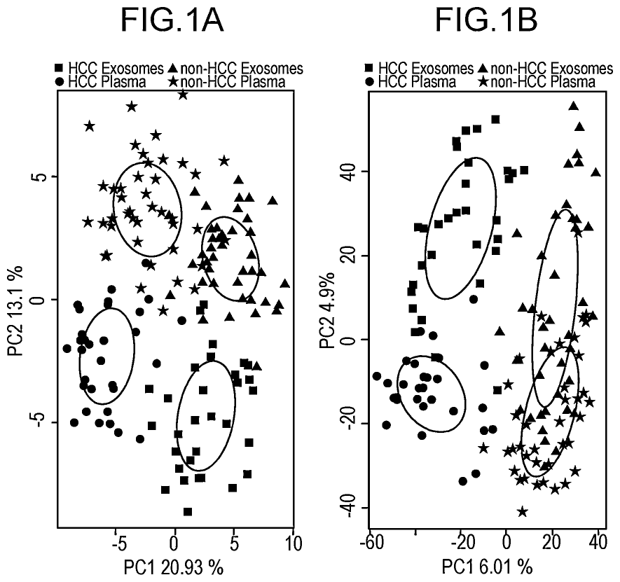

Non-targeted lipidomics was performed on all isolated exosomes and unfractionated plasma samples. After filtering to remove signals under background and species detected in less than 20% of the samples, a total of 2,864 lipid species belonging to 52 lipid classes, were identified. Among those 2,864 lipid species, 21 were detected only in exosomes and 75 only in plasma. The relative abundances of all 52 lipid classes in each group (exosomes HCC, exosomes non-HCC, plasma HCC, and plasma non-HCC), are summarized in Table 2. The two most abundant lipid classes were triglyceride (TG) and phosphatidylcholine (PC) with similar abundance of both classes in plasma (ratio TG/PC=0.93-0.98) but an enrichment of TG over PC in exosomes (ratio TG/PC=1.36-1.58). TG represented 53.5%-56.2% of all lipids in exosomes and 43.1%-43.5% in plasma. PC represented 35.5%-39.4% of all lipids in exosomes and 43.7%-46.8% in plasma. The third most abundant lipid class was sphingomyelin (SM) in all four groups (3.5%-6.2% of all lipids) (see FIGS. 1A-1B). The next three abundant classes were for all four groups, lysophosphatidylcholine (LPC), phosphatidylethanolamine (PE) and lysophosphatidic acid (LPA)). Sphingosine phosphate (SPHP) and cyclic phosphatidic acid (cPA) were only detected in unfractionated plasma. Sulfatide (ST) and acylGlcSitosterol ester (AcHexSiE) were only detected in non-HCC plasma and exosomes while dilysocardiolipin (DLCL) was only detected in HCC plasma and exosomes. These results are also represented in pie charts in FIG. 1 of Sanchez et al. (2021).

PCA was performed using the abundances of lipid classes (FIG. 1A) as well as species (FIG. 1B). Both exosome fractionation and HCC diagnosis had a significant impact on lipid profiles. Lipid classes clearly separated exosomes from plasma (p<0.001) and HCC from non-HCC (p<0.001) while lipid species clearly separated HCC from non-HCC (p<0.001) and HCC exosomes from the other three groups (p<0.001).

### 3. HCC-Associated Changes in Exosomal Lipid Classes and Species

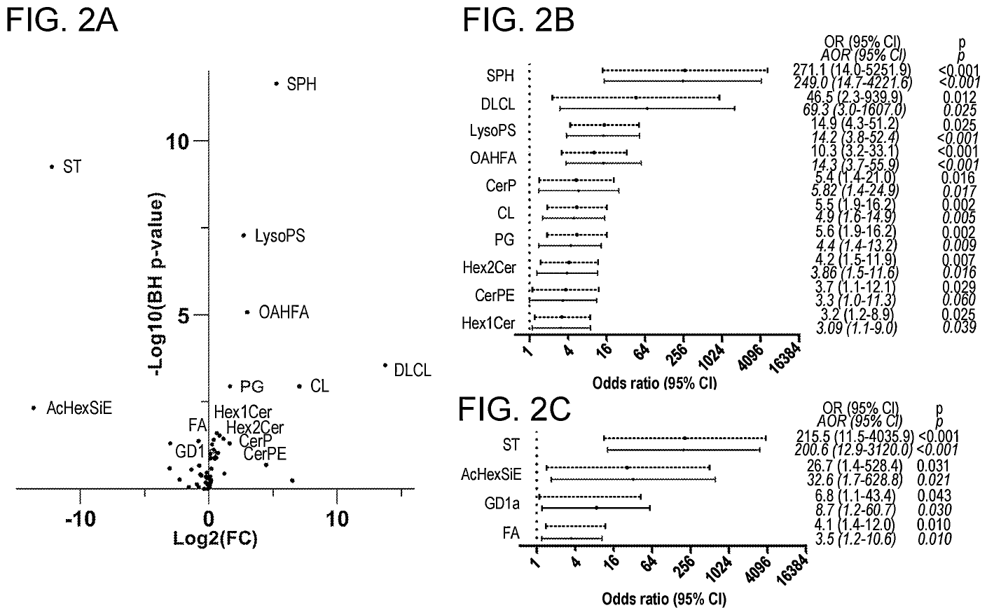

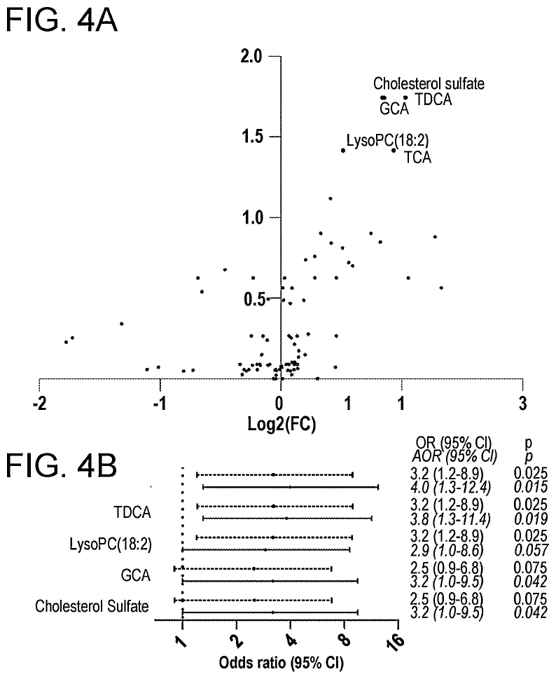

Ten lipid classes were found to be enriched in exosomes from HCC patients compared to exosomes in non-HCC patients (FIG. 2A). DLCL was detected in 35% of the HCC exosomes but in none of the control (non-HCC) exosomes (p<0.0001). Cardiolipin (CL) and sphingosine (SPH) had the highest differential effects with an FC of 133.08 (p<0.0001) and 38.57 (p<0.0001), respectively. The other enriched lipid classes were: (O-acyl)-1-hydroxy fatty acid (OAHFA) (FC=7.94, p<0.0001), lysophosphatidylserine (LysoPS) (FC=6.49, p<0.0001), phosphatidylglycerol (PG) (FC=3.16, p<0.0001), ceramide phosphoethanolamine (CerPE) (FC=3.05, p=0.049), ceramide phosphate (CerP) (FC=2.18, p=0.036), dihexosylceramide (Hex2Cer) (FC=1.78, p=0.029), and hexosylceramide (Hex1Cer) (FC=1.54, p=0.025). See FIG. 4A of Sanchez et al. (2021). Lipid species DLCL(16:0/20:3), CL(18:2/16:0/16:0/24:1), CL(18:2/18:0/18:0/24:1), CL(18:2/16:0/20:4/24:1), SPH(t18:0), OAHFA(18:2/32:0), LysoPS(34:1), PG(18:0/18:2), PG(16:0/18:2), CerPE(d18:1/16:0), CerP(m17:0/22:6), Hex2Cer(d15:0/18:2), Hex2Cer(d14:0/20:4), Hex1Cer(t20:0/18:2), and Hex1Cer(d18:1/22:0) were major contributors of the enrichment of these 10 lipid classes in HCC exosomes. In logistic regression analysis, high abundance (Tertile T3) of SPH, DLCL, LysoPS and OAHFA were strongly associated with HCC (OR [95% CI]: 271.1 [14.0-5251.9], p<0.001; 46.5 [2.3-939.9], p=0.012; 14.9 [4.3-51.2), p<0.001; 10.3 [3.2-33.1], p<0.001). The association remained significant after adjusting for age, gender and BMI (FIG. 2B).

Four lipid classes were depleted in HCC exosomes compared to non-HCC exosomes (FIGS. 2A-2C; see also FIG. 4B of Sanchez et al. (2021)). The abundance of lipid classes ganglioside (GD1a) and fatty acid (FA) were lower in HCC exosomes compared to non-HCC exosomes (FC=−8.05, p=0.049 and FC=−1.75, p=0.042, respectively). The lipid classes ST and AcHexSiE were undetectable in HCC exosomes but detected in 78% and 29% of non-HCC exosomes, respectively. Lipid species FA(20:4) GD1a(d18:1/18:0), GD1a(d18:1/16:0), ST(d18:1/20:2) and AcHexSiE(16:0) were major contributors of the depletion of these four lipid classes in HCC exosomes. See FIG. 4C of Sanchez et al. (2021). In logistic regression analysis, lack of detection of ST and ACHexSiE was strongly associated with HCC (OR [95% CI]: 215.5 [11.5-4035.9], p<0.001; 26.7 [1.4-528.4], p=0.031). The association remained significant after adjusting for age, gender and BMI (FIG. 2C).

A number of lipid species were detected in the majority of HCC exosomes but in none of the non-HCC exosomes (data not shown). These included PC(18:3e/22:4), PC(16:1e/22:6), SM(d14:0/23:1), CerG3GNAc1(t18:0/24:1), WE(26:5/18:0), SPH(t18:0), GM3(d18:1/22:0), TG(25:0/16:0/17:0), MGDG(16:0/21:6), TG(18:0/14:0/16:0), and DG(20:0/16:0). In contrast, the following lipid species were detected in a majority of non-HCC exosomes but in none of the HCC exosomes: PC(20:2e/18:1), PE(16:0/20:4), Hex1Cer(d16:0/26:2), TG(18:1/10:3/18:3), PE(20:0p/18:1), PC(18:1/24:2), LPA(10:0), PE(20:0p/20:3), ST(d18:1/20:2) and SM(t18:1/24:3). FIG. S2C shows depletion of species PC(18:1/24:2) PE(20:0p/20:3), and ST(d18:1/20:2).

### 4. Metabolic Profiling of Exosomes

Metabolomics was also performed on all isolated exosomes and unfractionated plasma samples. A total of 93 metabolites were identified after filtering against background and removing metabolites detected in less than 20% of the samples. Three of the four most abundant metabolites in exosomes were lysophospholipids (data not shown). Note: although lysophospholipids are lipids, we refer to them here as “metabolites” because they were detected using our metabolomics sample preparation and analytical workflow. LysoPC(16:0) was the most abundant metabolite in exosomes, representing 17.8% of all identified metabolites. The other lysophospholipids were LysoPC(18:2) (6.0%-7.9%) and LysoPC(18:0) (5.7%-5.8%). Those three lysophospholipids represented only 0.9-4.3% of all metabolites in plasma, suggesting a strong enrichment in exosomes. L-carnitine was the second most abundant metabolite, representing 7.6%-7.9% and 9.4%-10.4% of all identified metabolites in exosomes and plasma, respectively. Overall, the 20 most abundant metabolites were the same in all groups at the exception of LysoPC(18:0) and SM(d18:0/16:1) included in the top 20 only in exosomes and L-alanine and L-threonine included in the top 20 only in plasma. The relative abundance of all metabolites can be found in Table 3.

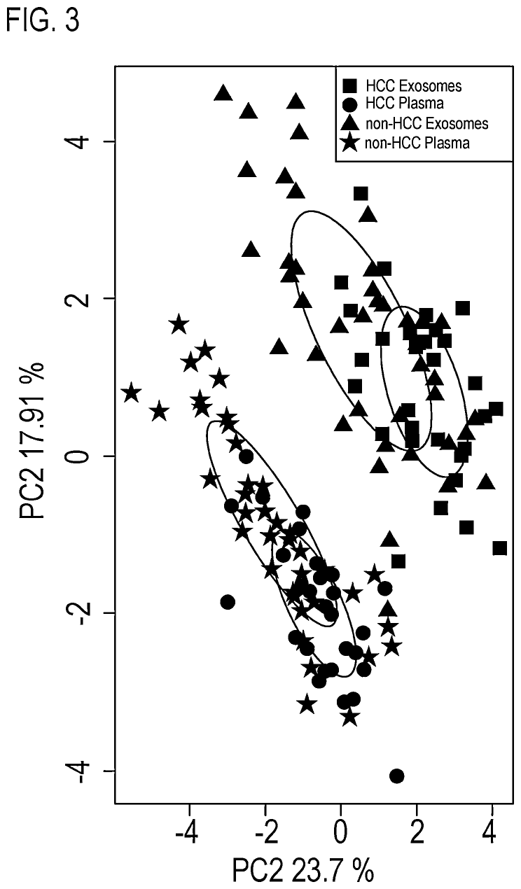

PCA was performed using the metabolites abundances normalized by internal standard and total signal (FIG. 3). Whereas exosome fractionation had a significant impact on metabolites profiles (p<0.0001), metabolites didn't fully separate HCC from non-HCC but the difference remained statistically significant (p<0.001).

### 5. HCC-Associated Changes in Exosomal Metabolites

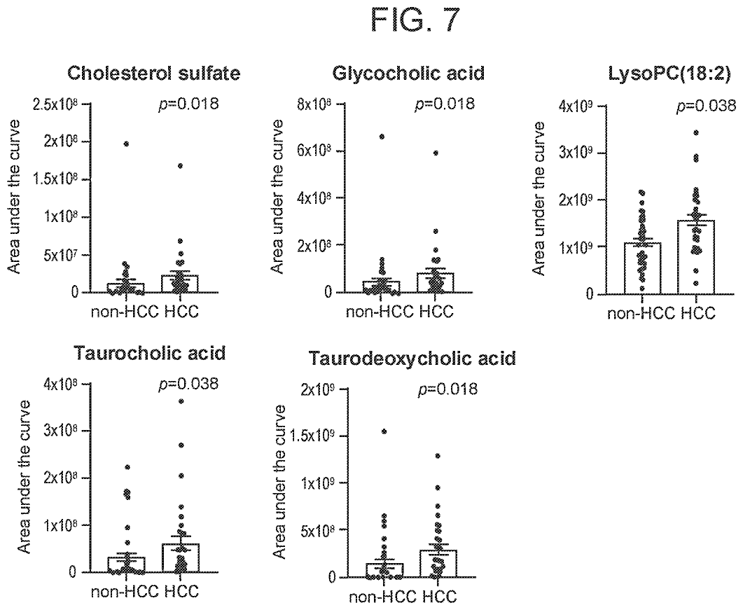

Only 5 metabolites were found to be significantly enriched in exosomes from HCC patients compared to exosomes in non-HCC patients. Three bile acids were among them: taurodeoxycholic acid (TDCA) (FC=2.04, p=0.018), taurocholic acid (TCA) (FC=1.91, p=0.038) and glycocholic acid (GCA) (FC=1.81, p=0.018). The other enriched metabolites were cholesterol sulfate (FC=1.79, p=0.018) and LysoPC(18:2) (FC=1.43, p=0.038) (FIG. 7). In logistic regression analysis, high abundance (T3) of all 5 metabolites were associated with HCC and the association remained significant after adjusting for age, gender and BMI (FIG. 4B).

6. Biological Pathways Associated with Lipid and Metabolite Changes in HCC Exosomes

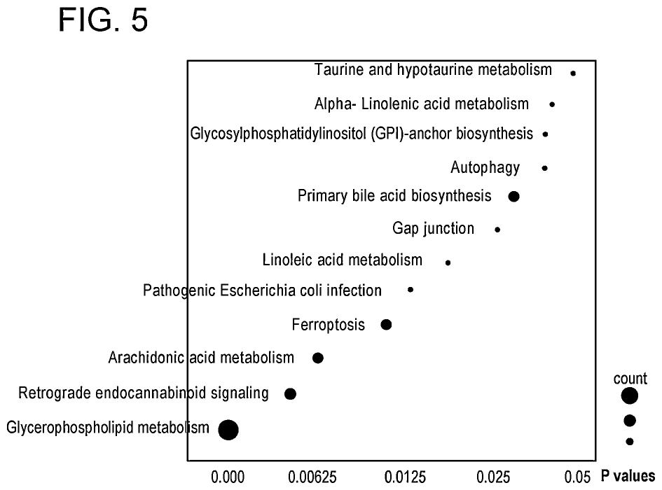

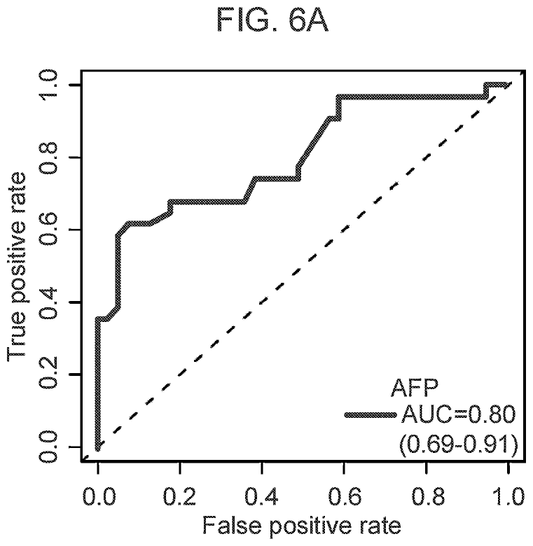

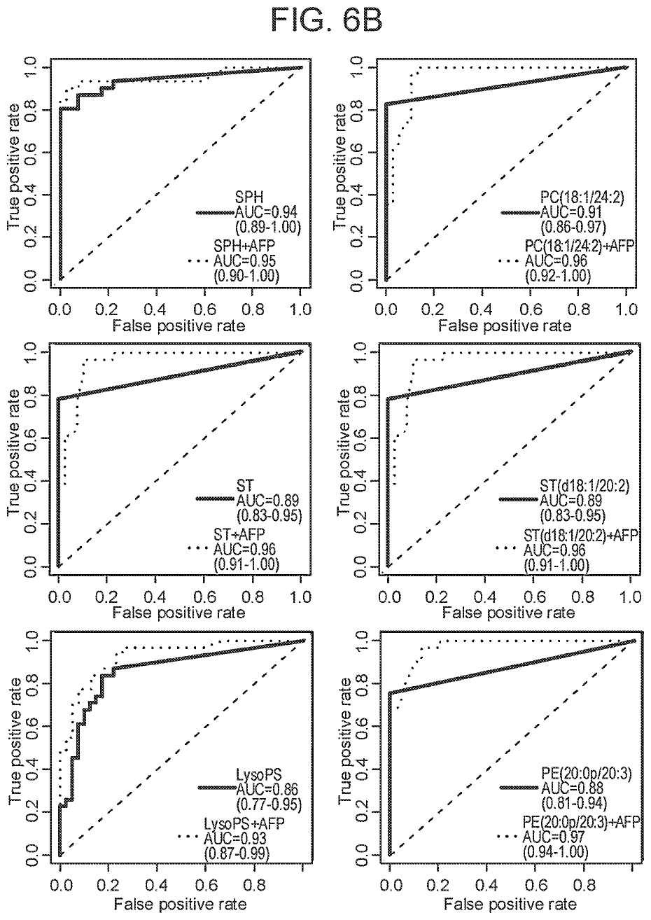

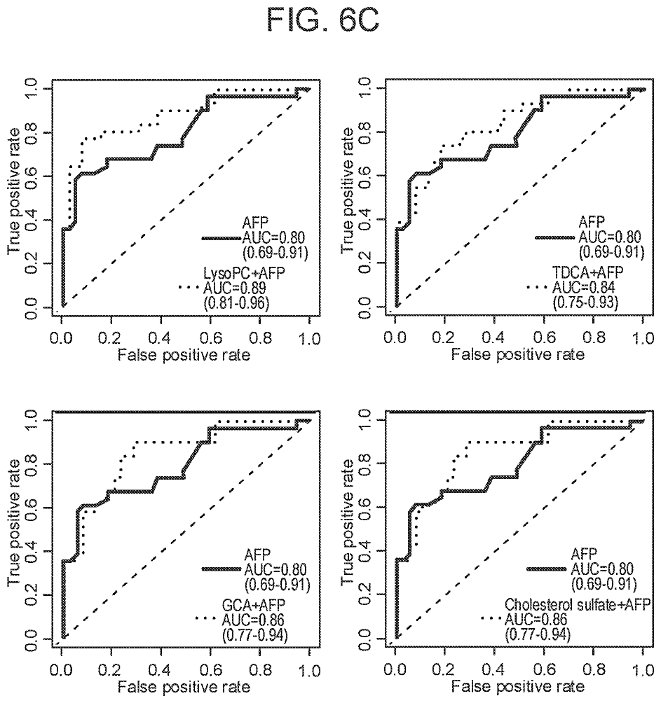

Pathway analysis using LIPEA and MetaboAnalyst identified 12 pathways impacted by the observed changes in lipids and metabolites in HCC exosomes compared to non-HCC exosomes. These included: glycerophospholipid metabolism (p<0.001), retrograde endocannabinoid signaling (p=0.005), arachidonic acid metabolism (p=0.007), ferroptosis (p=0.010), pathogenic Escherichia coli infection (p=0.015), linoleic acid metabolism (p=0.019), gap junction (p=0.030), primary bile acid biosynthesis (p=0.033), autophagy (p=0.044), glycosylphosphatidylinositol (GPI)-anchor biosynthesis (p=0.044), alpha-linolenic acid metabolism (p=0.049), and taurine and hypotaurine metabolism (p=0.05) (FIG. 5; see also FIG. 6 of Sanchez et al. (2021)).

### 7. Diagnostic Potential of Exosome Lipids and Metabolites

ROC curves were plotted using the lipid classes and lipid species identified as enriched or depleted in HCC exosomes compared to non-HCC exosomes and described above. As a reference, AUC for AFP was 0.80 [95% CI=0.69-0.91] (FIG. 6A). Among the lipid classes, three lipid classes in exosomes had significantly higher AUCs than AFP when used individually. These included SPH (AUC=0.94; 95% CI=0.89-1.00), ST (AUC=0.89, 95% CI=0.83-0.95) and LysoPS (AUC=0.86, 95% CI=0.77-0.95) (FIG. 6B). Their performance further increased when combined with AFP reaching 0.95 (95% CI=0.90-1.00) for SPH+AFP, 0.96 (95% CI=0.91-1.00) for ST+AFP and 0.93 (95% CI=0.87-0.99) for LysoPS+AFP (FIG. 6B). Among the lipid species, nine had higher AUCs than AFP when used individually, ranging from 0.82 to 0.91. The highest performance was observed for PC(18:1/24:2) (AUC=0.91, 95% CI=0.86-0.97), followed by ST(d18:1/20:2) (AUC=0.89, 95% CI=0.83-0.95) and PE(20:0p/20:3) (AUC=0.88, 95% CI=0.81-0.94) (FIG. 6B). AUCs increased for all nine lipids when combined with AFP ranging from 0.91 to 0.97. The highest performance was observed for PE(20:0p/20:3)+AFP (AUC=0.97, 95% CI=0.94-1.00) followed by PC(18:1/24:2)+AFP (AUC=0.96, 95% CI=0.92-1.00) and ST(d18:1/20:2)+AFP (AUC=0.96, 95% CI=0.91-1.00) (FIG. 6B).

ROC curves were also plotted using the five metabolites identified as enriched in HCC exosomes compared to non-HCC exosomes and described above. None of them had significantly higher AUCs than AFP when used individually. However, when combined with AFP, LysoPC(18:2), cholesterol sulfate and the two bile acids TDCA and GCA improved the performance of AFP (AUCs: 0.89 [95% CI=0.81-0.96], 0.86 [95% CI=0.77-0.94], 0.84 [95% CI=0.75-0.93] and 0.86 [95% CI=0.77-0.94], respectively) (FIG. 6C).

To further validate the AUC performances described above, we performed a 3-fold cross-validation analysis for all individual markers and their combination with AFP. The mean from the 3 runs showed similar AUCs (Table 4).

Sensitivity and specificity for detection of early HCC (BCLC 0 and A) were calculated for the lipids and metabolites described above and their combination with AFP (Table 4). At 20 ng/ml, AFP's performance was 45% sensitivity at 95% specificity. The combination of AFP+SPH reached 90% sensitivity at 97% specificity and the combination of AFP+ST reached 95% sensitivity at 90% specificity.

### C. Discussion

Most studies on the utility of exosomes for diagnosis have focused on proteins and miRNAs. In this study, we used ultra-high resolution mass spectrometry to identify lipid and metabolite differences between exosomes isolated from cirrhotic patients with and without HCC. An important strength of our approach was that the Orbitrap technology permitted identification of thousands of metabolites and lipids. A limitation of the workflow, however, is one that plagues the entire metabolomics field—annotation confidence. Although the combination of high scan speed and ultra-high resolution permitted acquisition of MS² spectra for thousands of metabolites and lipids, to perform database matching and subsequent annotation, confirmation of those annotations would ultimately require retention time matching, which is not possible using conventional non-targeted profiling workflows. Nevertheless, our analysis elucidated a number of novel associations with both exosome isolation and HCC diagnosis having significant impact on lipid and metabolite profiles.

Ten lipid classes were enriched in exosomes from cirrhotic patients with HCC compared to exosomes from cirrhotic patients without HCC. Among them, SPH had among the highest differential abundance and highest diagnostic performance when used alone or in combination with AFP. Most remarkably, the combination of AFP+SPH reached 90% sensitivity at 97% specificity for the detection of early HCC (BCLC 0 and A), compared to 45% sensitivity at 95% specificity for AFP at 20 ng/ml. SPH(t18:0) was the main lipid responsible for this effect. The phosphorylated form of SPH, SPH-1P, has been shown to regulate hepatocyte exosome-dependent liver repair and regeneration (34). Furthermore, exosome adherence and internalization by hepatic stellate cells trigger SPH-1P dependent migration (35). SPH(d18:1)-1P has been proposed as a serum biomarker for HCC in patients with cirrhosis (36) and as a risk marker for HCC in a large population-based cohort (37). A second exosomal lipid class that had strong diagnostic performance for HCC was ST. ST was detected in 78% of non-HCC exosomes but undetectable in HCC exosomes. The combination of AFP+ST reached 95% sensitivity at 90% specificity for the detection of early HCC (BCLC 0 and A). ST has specific anti-inflammatory and immunomodulatory properties (38). ST reactive-type II NKT cells are immunosuppressive in inflammatory liver diseases and attenuate alcoholic liver disease in mice (39). We also detected LysoPS at significantly higher levels in HCC exosomes compared to non-HCC exosomes. LysoPS(34:1) was the main form responsible for that increase but this specific LysoPS form remains largely uncharacterized. Recent studies have revealed important roles for LysoPS signaling in T cell and macrophage functions (40, 41).

Only 5 metabolites were found to be enriched in exosomes from HCC patients compared to exosomes in cirrhotic patients without HCC. Three bile acids were among them. Bile acids TDCA and GCA improved the performance of AFP. The liver is central to bile acid metabolism, thus a change in bile acid profiles may be among the earliest indicators of HCC development. We and others have reported increases in TDCA and GCA serum levels with disease progression from fibrosis to HCC. Hepatic levels of TDCA and GCA substantially increase in mice at the occurrence of liver fibrosis and bile acid TDCA positively correlates with gut microbiota changes of alistipes and parabacteroides (42). GCA and TDCA displayed strong associations with fibrosis in a Mexican-American population with high incidence of HCC (43). GCA levels positively associated with HCC risk in the general population as well as in HBV subjects (37, 44, 45).

In addition to the potential use of the identified lipids and metabolites in HCC early detection in cirrhotic patients, pathway analysis identified 12 pathways impacted by the observed changes in lipids and metabolites in HCC exosomes compared to non-HCC exosomes. Greater impact was predicted on glycerophospholipid metabolism, arachidonic acid metabolism, ferroptosis and primary bile acid biosynthesis. Arachidonic acid metabolism has been associated with hepatocarcinogenesis (46, 47). Interestingly, ferroptosis, a new recognized way of non-apoptosis-regulated cell death characterized by the iron-dependent accumulation of lipid peroxides, shows promise in the therapy of cancer, especially in HCC (48, 49). Whether circulating exosomes by altering glycerophospholipid metabolism, arachidonic acid metabolism, ferroptosis or primary bile acid biosynthesis, contribute to the development of HCC should be further investigated.

Altogether, this study identified promising biomarkers for the detection of early stage HCC in at-risk cirrhosis patients and confirmed the promise of using exosomes as shown in the recently published analysis of purified extracellular vesicles combined to reverse transcription (50). In addition, this study identified pathways altered in HCC exosomes that may contribute to tumor development and progression.

TABLE 2
Relative abundance (%) of the lipid classes detected in non-HCC exosomes, HCC
exosomes, non-HCC plasma and HCC plasma. Data are shown as mean (range).

| | non-HCC exosomes | HCC exosomes | non-HCC plasma | HCC plasma |
|---|---|---|---|---|
| Acyl Carnitine (AcCa) | 0.006 (0.000-0.029) | 0.007 (0.000-0.042) | 0.017 (0.002-0.123) | 0.016 (0.002-0.053) |
| AcylGlcSitosterol Ester | 0.000 (0.000-0.004) | Undetected | 0.000 (0.000-0.002) | Undetected |
| (AcHexSiE) | | | | |
| Ceramides (Cer) | 0.080 (0.013-0.262) | 0.088 (0.044-0.266) | 0.080 (0.022-0.213) | 0.074 (0.032-0.173) |
| Simple Glc Series (CerG3GNAc1) | 0.011 (0.000-0.025) | 0.009 (0.002-0.037) | 0.013 (0.001-0.039) | 0.011 (0.003-0.025) |
| Simple Glc Series (CerG3GNAc2) | 0.000 (0.000-0.001) | 0.000 (0.000-0.001) | 0.000 (0.000-0.001) | 0.000 (0.000-0.001) |
| Ceramides Phosphate (CerP) | 0.000 (0.000-0.005) | 0.000 (0.000-0.002) | 0.000 (0.000-0.002) | Undetected |
| Ceramide Phosphoethanolamines | 0.000 (0.000-0.002) | 0.000 (0.000-0.001) | 0.000 (0.000-0.001) | 0.001 (0.000-0.002) |
| (CerPE) | | | | |
| Cholesteryl Ester (ChE) | 0.407 (0.046-1.430) | 0.374 (0.174-0.821) | 0.641 (0.084-1.323) | 0.683 (0.233-1.225) |
| Cardiolipin (CL) | 0.001 (0.000-0.023) | 0.164 (0.000-1.175) | 0.013 (0.000-0.474) | 0.106 (0.000-0.725) |
| Campesterol Ester (CmE) | 0.000 (0.000-0.004) | 0.000 (0.000-0.002) | 0.001 (0.000-0.007) | 0.001 (0.000-0.005) |
| Coenzyme (Co) | 0.012 (0.000-0.048) | 0.008 (0.000-0.014) | 0.012 (0.000-0.037) | 0.009 (0.004-0.017) |
| cyclic Phosphatidic Acid (cPA) | Undetected | Undetected | 0.004 (0.000-0.018) | 0.011 (0.000-0.041) |
| Deuterated Cholesteryl Ester | 0.002 (0.000-0.007) | 0.003 (0.000-0.006) | 0.002 (0.000-0.004) | 0.001 (0.000-0.003) |
| (D7ChE) | | | | |
| Diglyceride (DG) | 0.298 (0.053-2.053) | 0.351 (0.110-1.641) | 0.310 (0.092-0.891) | 0.691 (0.150-1.948) |
| Dilysocardiolipin (DLCL) | Undetected | 0.000 (0.000-0.003) | Undetected | 0.000 (0.000-0.002) |
| Fatty Acid (FA) | 0.001 (0.000-0.008) | 0.001 (0.000-0.009) | 0.005 (0.000-0.022) | 0.004 (0.000-0.015) |
| Gangliosides (GD1a) | 0.000 (0.000-0.001) | 0.000 (0.000-0.001) | 0.000 (0.000-0.003) | 0.000 (0.000-0.002) |
| Gangliosides (GD3) | 0.001 (0.000-0.002) | 0.001 (0.000-0.001) | 0.001 (0.000-0.002) | 0.001 (0.000-0.003) |
| Gangliosides (GM2) | 0.000 (0.000-0.002) | 0.000 (0.000-0.002) | 0.000 (0.000-0.000) | 0.000 (0.000-0.000) |
| Gangliosides (GM3) | 0.020 (0.005-0.058) | 0.018 (0.002-0.042) | 0.025 (0.008-0.052) | 0.026 (0.014-0.047) |
| Simple Glc series (Hex1Cer) | 0.130 (0.022-0.471) | 0.198 (0.045-0.674) | 0.178 (0.034-0.568) | 0.280 (0.112-0.470) |
| Simple Glc series (Hex2Cer) | 0.032 (0.004-0.072) | 0.056 (0.012-0.220) | 0.040 (0.006-0.098) | 0.064 (0.030-0.159) |
| Simple Glc series (Hex3Cer) | 0.011 (0.003-0.032) | 0.018 (0.002-0.083) | 0.015 (0.001-0.037) | 0.021 (0.006-0.044) |
| Lysophosphatidic Acid (LPA) | 0.475 (0.169-1.535) | 0.484 (0.140-1.292) | 0.689 (0.155-1.484) | 0.981 (0.227-2.491) |
| Lysophosphatidylcholine (LPC) | 0.670 (0.059-1.939) | 0.562 (0.130-1.525) | 1.353 (0.302-3.133) | 1.713 (0.268-4.984) |
| Lysophosphatidylethanolamine | 0.011 (0.002-0.057) | 0.014 (0.003-0.042) | 0.022 (0.008-0.117) | 0.028 (0.006-0.072) |
| (LPE) | | | | |
| Lysophosphatidylglycerol (LPG) | Undetected | 0.000 (0.000-0.000) | 0.002 (0.000-0.004) | 0.002 (0.000-0.008) |
| Lysophosphatidylinositol (LPI) | 0.000 (0.000-0.002) | 0.001 (0.000-0.005) | 0.006 (0.001-0.013) | 0.009 (0.000-0.024) |
| Lysophosphatidylserine (LPS) | 0.000 (0.000-0.002) | 0.001 (0.000-0.004) | 0.000 (0.000-0.002) | 0.001 (0.000-0.002) |
| Lysosphingomyelin (LSM) | 0.001 (0.000-0.005) | 0.001 (0.000-0.006) | 0.001 (0.000-0.003) | 0.001 (0.000-0.002) |
| Monoglyceride (MG) | 0.002 (0.000-0.023) | 0.001 (0.000-0.009) | 0.003 (0.000-0.019) | 0.000 (0.000-0.003) |
| Monogalactosyldiacylglycerol | 0.052 (0.000-0.194) | 0.058 (0.000-0.369) | 0.060 (0.000-0.155) | 0.071 (0.016-0.299) |
| (MGDG) | | | | |
| Monogalactosylmonoacylglycerol | 0.000 (0.000-0.003) | 0.000 (0.000-0.002) | 0.000 (0.000-0.003) | 0.000 (0.000-0.004) |
| (MGMG) | | | | |
| Monolysocardiolipin (MLCL) | 0.045 (0.000-0.317) | 0.023 (0.001-0.192) | 0.190 (0.000-0.664) | 0.198 (0.026-0.991) |
| (O-acyl)-1-hydroxy fatty acid | 0.000 (0.000-0.000) | 0.000 (0.000-0.001) | 0.000 (0.000-0.000) | 0.000 (0.000-0.001) |
| (OAHFA) | | | | |
| Phosphatidylcholine (PC) | 39.41 (16.60-64.88) | 35.52 (15.00-73.65) | 46.79 (22.66-63.36) | 43.69 (30.77-63.10) |
| Phosphatidylethanolamine (PE) | 0.657 (0.257-1.374) | 0.570 (0.187-1.105) | 0.980 (0.322-2.162) | 0.937 (0.372-1.762) |
| Phosphatidylethanol (Pet) | 0.000 (0.000-0.003) | 0.004 (0.000-0.030) | 0.014 (0.000-0.068) | 0.010 (0.000-0.088) |
| Phosphatidylglycerol (PG) | 0.000 (0.000-0.003) | 0.001 (0.000-0.002) | 0.008 (0.000-0.057) | 0.001 (0.000-0.037) |
| Phytosphingosine (phSM) | 0.370 (0.000-1.388) | 0.411 (0.000-2.911) | 0.424 (0.000-1.606) | 0.565 (0.000-2.169) |
| Phosphatidylinositol (PI) | 0.221 (0.062-0.622) | 0.251 (0.000-0.475) | 0.373 (0.004-1.125) | 0.380 (0.154-0.840) |
| phosphatidylinositol-P2 (PIP2) | 0.000 (0.000-0.001) | 0.000 (0.000-0.000) | 0.000 (0.000-0.002) | 0.000 (0.000-0.001) |
| phosphatidylserine (PS) | 0.022 (0.000-0.161) | 0.024 (0.004-0.065) | 0.009 (0.000-0.056) | 0.006 (0.000-0.037) |
| Sitosteryl ester (SiE) | 0.000 (0.000-0.002) | 0.000 (0.000-0.001) | 0.000 (0.000-0.003) | 0.001 (0.000-0.002) |
| Sphingomyelin (SM) | 3.513 (1.18-11.48) | 4.551 (0.981-10.22) | 4.116 (1.71-11.04) | 6.178 (2.88-17.30) |
| Sphingosine (SPH) | 0.000 (0.000-0.003) | 0.011 (0.000-0.057) | 0.000 (0.000-0.006) | 0.004 (0.000-0.013) |
| Sphingosine phosphate (SPHP) | Undetected | Undetected | 0.001 (0.000-0.008) | 0.001 (0.000-0.004) |
| Sulfatide (ST) | 0.000 (0.000-0.001) | Undetected | 0.000 (0.000-0.002) | Undetected |
| Stigmasteryl ester (StE) | 0.009 (0.000-0.047) | 0.005 (0.000-0.026) | 0.018 (0.000-0.044) | 0.018 (0.000-0.045) |
| Triglyceride (TG) | 53.49 (28.93-79.20) | 56.18 (19.71-81.40) | 43.52 (26.88-73.06) | 43.13 (23.20-58.83) |
| Wax Esters (WE) | 0.028 (0.000-0.095) | 0.018 (0.003-0.051) | 0.052 (0.007-0.119) | 0.050 (0.012-0.174) |
| Zymosteryl (ZyE) | 0.009 (0.000-0.046) | 0.007 (0.000-0.021) | 0.013 (0.001-0.035) | 0.015 (0.000-0.029) |

TABLE 3
Relative abundance (%) of metabolites detected in non-HCC exosomes, HCC
exosomes, non-HCC plasma and HCC plasma. Data are shown as mean (range).

| | non-HCC exosomes | | HCC exosomes | | non-HCC plasma | | HCC plasma | |
|---|---|---|---|---|---|---|---|---|
| 1-(Hydroxymethyl)-5,5-dimethyl- | 0.460 | (0.173-1.37) | 0.372 | (0.161-0.599) | 0.707 | (0.288-1.22) | 0.832 | (0.308-1.38) |
| 2,4-imidazolidinedione | | | | | | | | |
| 1-Methylhistidine | 0.242 | (0.048-0.929) | 0.302 | (0.036-3.10) | 0.715 | (0.179-3.40) | 0.523 | (0.003-1.10) |
| 3′,4′-Dihydrodiol | 0.071 | (0.003-0.660) | 0.026 | (0.001-0.105) | 0.017 | (0.000-0.065) | 0.016 | (0.001-0.133) |
| 4-Hydroxyproline | 0.340 | (0.073-1.02) | 0.322 | (0.159-0.640) | 0.873 | (0.272-1.39) | 1.045 | (0.439-1.66) |
| 5-Acetylamino-6-formylamino-3- | 1.404 | (0.462-4.29) | 1.380 | (0.657-4.41) | 3.675 | (1.80-9.00) | 3.233 | (2.06-8.76) |
| methyluracil | | | | | | | | |
| 5-Hydroxyisourate | 0.025 | (0.001-0.166) | 0.018 | (0.001-0.064) | 0.097 | (0.002-0.649) | 0.049 | (0.006-0.652) |
| Acetyl-N-formyl-5- | 0.252 | (0.005-4.06) | 0.067 | (0.003-0.239) | 0.222 | (0.007-1.74) | 0.124 | (0.014-0.494) |
| methoxykynurenamine | | | | | | | | |
| alanyl-histidine | 0.043 | (0.000-0.339) | 0.028 | (0.000-0.108) | 0.016 | (0.000-0.113) | 0.022 | (0.000-0.144) |
| Alanyl-Serine | 0.060 | (0.020-0.161) | 0.046 | (0.006-0.126) | 0.084 | (0.016-0.363) | 0.116 | (0.032-0.785) |
| Allantoin | 0.028 | (0.007-0.069) | 0.031 | (0.014-0.063) | 0.041 | (0.016-0.072) | 0.046 | (0.023-0.082) |
| Benzoic acid | 0.035 | (0.009-0.094) | 0.031 | (0.007-0.062) | 0.038 | (0.007-0.118) | 0.047 | (0.019-0.116) |
| CDP-glycerol | 0.003 | (0.000-0.015) | 0.004 | (0.000-0.013) | 0.005 | (0.000-0.016) | 0.043 | (0.006-0.243) |
| Cholesterol sulfate | 0.069 | (0.000-1.28) | 0.112 | (0.009-0.769) | 0.087 | (0.001-0.453) | 0.104 | (0.003-0.537) |
| cis-Aconitic acid | 0.242 | (0.078-0.477) | 0.258 | (0.125-0.571) | 0.300 | (0.129-0.498) | 0.302 | (0.059-1.19) |
| Citrulline | 0.638 | (0.224-1.99) | 0.507 | (0.215-0.869) | 1.014 | (0.402-1.75) | 1.191 | (0.392-1.97) |
| Creatine | 0.363 | (0.029-1.04) | 0.319 | (0.082-1.75) | 0.429 | (0.052-1.47) | 0.391 | (0.076-1.21) |
| Creatinine | 3.979 | (1.46-16.51) | 3.822 | (2.12-11.15) | 5.443 | (2.64-21.81) | 4.851 | (2.31-8.97) |
| Cytidine | 0.017 | (0.003-0.037) | 0.017 | (0.006-0.027) | 0.041 | (0.017-0.090) | 0.033 | (0.009-0.072) |
| Deoxycholic acid glycine conjugate | 0.719 | (0.013-2.64) | 0.900 | (0.145-2.45) | 0.233 | (0.006-0.726) | 0.322 | (0.052-0.694) |
| D-Glucose | 2.476 | (0.859-7.73) | 2.484 | (1.10-8.09) | 5.45 | (2.58-12.88) | 4.739 | (2.97-13.77) |
| D-Urobilinogen | 0.060 | (0.000-0.624) | 0.096 | (0.000-0.779) | 0.043 | (0.000-0.291) | 0.061 | (0.000-0.502) |
| Fenamiphos | 0.059 | (0.013-0.141) | 0.057 | (0.017-0.100) | 0.190 | (0.075-0.407) | 0.153 | (0.040-0.331) |
| Flavone | 0.007 | (0.000-0.071) | 0.009 | (0.000-0.037) | 0.019 | (0.002-0.155) | 0.018 | (0.004-0.082) |
| Fructoseglycine | 0.003 | (0.000-0.017) | 0.002 | (0.000-0.011) | 0.007 | (0.001-0.032) | 0.007 | (0.001-0.021) |
| Glycerophosphocholine | 0.212 | (0.046-0.794) | 0.153 | (0.077-0.245) | 1.256 | (0.215-2.78) | 1.019 | (0.198-2.964) |
| Glycocholic acid | 0.244 | (0.003-4.28) | 0.401 | (0.039-2.71) | 0.303 | (0.004-1.59) | 0.360 | (0.011-1.84) |
| Guanidoacetic acid | 0.022 | (0.001-0.110) | 0.020 | (0.005-0.047) | 0.031 | (0.001-0.191) | 0.058 | (0.002-0.198) |
| Homo-L-arginine | 0.080 | (0.006-0.918) | 0.078 | (0.012-0.260) | 0.097 | (0.001-0.352) | 0.144 | (0.025-0.425) |
| Hypotaurine | 0.003 | (0.000-0.014) | 0.003 | (0.000-0.008) | 0.007 | (0.000-0.021) | 0.007 | (0.001-0.017) |
| Hypoxanthine | 0.213 | (0.032-0.507) | 0.259 | (0.093-0.610) | 0.198 | (0.054-0.487) | 0.216 | (0.057-0.489) |
| Indoleacrylic acid | 0.880 | (0.194-2.39) | 0.827 | (0.138-2.05) | 0.444 | (0.110-1.11) | 0.406 | (0.213-0.623) |
| Indoxyl sulfate | 0.439 | (0.001-5.84) | 0.185 | (0.000-1.03) | 0.483 | (0.001-10.75) | 0.130 | (0.001-0.668) |
| L-Alanine | 1.137 | (0.523-2.41) | 1.087 | (0.346-2.23) | 1.867 | (0.470-3.02) | 1.924 | (0.750-3.64) |
| L-Arginine | 3.604 | (1.23-9.63) | 3.481 | (1.53-5.82) | 4.331 | (1.217-9.40) | 5.092 | (2.09-7.66) |
| L-Asparagine | 0.459 | (0.199-1.17) | 0.404 | (0.180-0.808) | 0.672 | (0.336-1.19) | 0.747 | (0.404-1.32) |
| L-Aspartic acid | 0.492 | (0.028-3.22) | 0.222 | (0.025-1.04) | 0.057 | (0.022-0.102) | 0.051 | (0.026-0.120) |
| L-Carnitine | 7.557 | (3.09-13.54) | 7.933 | (5.40-12.04) | 10.420 | (3.11-15.22) | 9.390 | (5.92-13.36) |
| L-Cysteine | 0.010 | (0.004-0.025) | 0.010 | (0.004-0.022) | 0.016 | (0.008-0.029) | 0.012 | (0.005-0.027) |
| L-Cysteinylglycine disulfide | 0.332 | (0.135-0.680) | 0.258 | (0.132-0.524) | 0.483 | (0.208-1.04) | 0.373 | (0.165-0.901) |
| L-Cystine | 1.386 | (0.714-2.97) | 1.167 | (0.538-2.45) | 1.851 | (1.03-3.14) | 1.468 | (0.657-3.91) |
| Lenticin | 0.118 | (0.000-1.614) | 0.109 | (0.003-0.394) | 0.117 | (0.001-1.28) | 0.101 | (0.002-0.376) |
| leucyl-proline | 0.325 | (0.065-1.27) | 0.320 | (0.120-2.04) | 0.491 | (0.114-2.37) | 0.422 | (0.185-1.202) |
| L-Glutamic acid | 0.959 | (0.259-4.39) | 0.764 | (0.279-1.94) | 0.979 | (0.255-4.51) | 0.999 | (0.252-2.60) |
| L-Glutamine | 4.919 | (1.86-12.14) | 4.509 | (2.53-6.90) | 8.585 | (5.15-11.70) | 9.025 | (5.81-12.70) |
| L-Kynurenine | 0.027 | (0.003-0.093) | 0.023 | (0.003-0.068) | 0.054 | (0.015-0.122) | 0.048 | (0.018-0.096) |
| L-Leucine | 4.386 | (1.06-11.50) | 3.547 | (1.39-6.68) | 5.553 | (1.40-11.79) | 5.401 | (2.36-11.09) |
| L-Lysine | 2.389 | (0.797-6.14) | 1.810 | (0.552-3.24) | 3.332 | (1.105-5.04) | 3.220 | (1.36-5.78) |
| L-Methionine | 0.104 | (0.018-0.560) | 0.089 | (0.017-0.215) | 0.490 | (0.137-1.17) | 0.659 | (0.269-1.236) |
| L-Phenylalanine | 1.640 | (0.615-3.79) | 1.704 | (0.743-2.72) | 2.021 | (0.847-4.52) | 2.707 | (1.52-4.66) |
| L-Proline | 1.512 | (0.900-2.58) | 1.392 | (0.615-3.27) | 2.023 | (0.874-3.21) | 1.937 | (0.798-3.82) |
| L-Serine | 1.566 | (0.248-8.34) | 0.862 | (0.197-3.03) | 0.682 | (0.282-1.09) | 0.677 | (0.297-1.03) |
| L-Threonine | 1.254 | (0.473-3.41) | 0.915 | (0.200-1.51) | 1.652 | (0.680-3.58) | 1.685 | (0.697-2.80) |
| L-Tryptophan | 1.001 | (0.220-2.75) | 0.942 | (0.138-2.34) | 0.495 | (0.145-1.29) | 0.456 | (0.244-0.728) |
| L-Tyrosine | 0.498 | (0.104-1.317) | 0.454 | (0.129-0.894) | 0.517 | (0.105-1.47) | 0.621 | (0.266-1.50) |
| L-Valine | 3.513 | (1.81-7.86) | 3.680 | (1.80-11.55) | 4.571 | (1.17-11.80) | 4.562 | (1.65-14.22) |
| LysoPC(15:0) | 0.358 | (0.069-0.853) | 0.361 | (0.098-0.560) | 0.072 | (0.020-0.247) | 0.061 | (0.029-0.115) |
| LysoPC(16:0) | 17.772 | (4.87-29.23) | 17.800 | (5.41-27.15) | 4.330 | (1.91-8.40) | 3.695 | (1.93-6.30) |
| LysoPC(16:1(9Z)) | 0.792 | (0.216-2.85) | 0.778 | (0.281-2.46) | 0.277 | (0.085-0.980) | 0.202 | (0.083-0.509) |
| LysoPC(18:0) | 5.798 | (1.57-12.09) | 5.721 | (1.52-9.68) | 0.870 | (0.121-2.13) | 0.891 | (0.37-1.80) |
| LysoPC(18:2(9Z,12Z)) | 6.042 | (1.20-11.57) | 7.861 | (1.98-14.62) | 2.397 | (1.38-4.01) | 2.305 | (1.07-4.88) |
| LysoPE(0:0/18:2(9Z,12Z)) | 0.692 | (0.075-1.58) | 0.897 | (0.244-2.27) | 0.201 | (0.059-0.425) | 0.233 | (0.057-0.722) |
| Methionine sulfoxide | 0.047 | (0.011-0.158) | 0.039 | (0.014-0.082) | 0.061 | (0.026-0.117) | 0.087 | (0.033-0.208) |
| Miglitol | 0.072 | (0.023-0.162) | 0.083 | (0.045-0.154) | 0.124 | (0.020-0.249) | 0.095 | (0.019-0.179) |
| N-(3-acetamidopropyl)pyrrolidin- | 0.064 | (0.005-0.214) | 0.072 | (0.011-0.519) | 0.114 | (0.010-0.347) | 0.117 | (0.017-0.449) |
| 2-one | | | | | | | | |
| N(6)-Methyllysine | 0.106 | (0.023-0.402) | 0.090 | (0.019-1.13) | 0.213 | (0.039-0.822) | 0.160 | (0.056-0.812) |
| N6,N6,N6-Trimethyl-L-lysine | 0.065 | (0.012-0.208) | 0.063 | (0.013-0.308) | 0.089 | (0.035-0.205) | 0.097 | (0.042-0.343) |
| N-a-Acetylcitrulline | 0.004 | (0.000-0.023) | 0.008 | (0.001-0.059) | 0.005 | (0.000-0.046) | 0.008 | (0.001-0.063) |
| N-Acetylornithine | 0.028 | (0.005-0.160) | 0.027 | (0.006-0.068) | 0.058 | (0.013-0.163) | 0.088 | (0.020-0.281) |
| Ne,Ne dimethyllysine | 0.029 | (0.003-0.174) | 0.027 | (0.003-0.534) | 0.049 | (0.013-0.193) | 0.041 | (0.019-0.153) |
| Norfuraneol | 0.134 | (0.044-0.379) | 0.134 | (0.073-0.395) | 0.351 | (0.162-0.901) | 0.289 | (0.174-0.819) |
| Norophthalmic acid | 0.045 | (0.000-0.296) | 0.026 | (0.000-0.094) | 0.194 | (0.042-0.486) | 0.206 | (0.046-0.514) |
| Ornithine | 1.298 | (0.309-7.78) | 0.751 | (0.121-1.70) | 0.919 | (0.389-1.76) | 0.950 | (0.460-1.80) |
| Orotidine | 0.027 | (0.000-0.220) | 0.007 | (0.000-0.064) | 0.006 | (0.001-0.060) | 0.004 | (0.001-0.013) |
| Pantothenic acid | 0.008 | (0.002-0.032) | 0.010 | (0.004-0.020) | 0.010 | (0.002-0.038) | 0.013 | (0.004-0.040) |
| Phenylpyruvic acid | 0.077 | (0.015-0.222) | 0.068 | (0.020-0.143) | 0.084 | (0.014-0.255) | 0.101 | (0.041-0.250) |
| Piperidine | 0.627 | (0.156-1.64) | 0.506 | (0.181-0.964) | 0.753 | (0.233-1.64) | 0.713 | (0.314-1.43) |
| Proline betaine | 1.302 | (0.037-9.26) | 2.874 | (0.062-15.60) | 1.862 | (0.021-11.28) | 3.436 | (0.050-17.73) |
| Pyroglutamic acid | 3.753 | (1.49-9.02) | 3.433 | (1.88-4.96) | 6.195 | (3.82-8.41) | 6.478 | (4.19-9.01) |
| S-Adenosylhomocysteine | 0.001 | (0.000-0.009) | 0.001 | (0.000-0.011) | 0.002 | (0.000-0.015) | 0.002 | (0.001-0.003) |
| Sedoheptulose | 0.449 | (0.021-1.35) | 0.445 | (0.045-1.07) | 0.507 | (0.033-1.35) | 0.514 | (0.025-1.31) |
| SM(d18:0/16:1(9Z)) | 2.710 | (0.031-6.32) | 3.721 | (0.341-8.80) | 0.105 | (0.002-0.455) | 0.124 | (0.009-0.423) |
| S-methyl-5-thio-D-ribulose 1- | 0.081 | (0.017-0.161) | 0.078 | (0.032-0.298) | 0.166 | (0.055-0.260) | 0.161 | (0.088-0.257) |
| phosphate(2-) | | | | | | | | |
| Sphingosine 1-phosphate | 0.244 | (0.070-1.010) | 0.216 | (0.094-0.465) | 0.042 | (0.014-0.081) | 0.039 | (0.018-0.085) |
| Sulfoglycolithocholate(2-) | 0.017 | (0.001-0.112) | 0.040 | (0.002-0.721) | 0.016 | (0.001-0.065) | 0.027 | (0.003-0.075) |
| Taurine | 0.114 | (0.042-0.272) | 0.099 | (0.052-0.209) | 0.710 | (0.224-1.64) | 0.472 | (0.214-1.014) |
| Taurocholic acid | 0.176 | (0.000-1.316) | 0.306 | (0.003-1.66) | 0.221 | (0.000-1.70) | 0.424 | (0.006-2.559) |
| Taurodeoxycholic acid | 0.793 | (0.001-9.43) | 1.475 | (0.032-5.55) | 0.491 | (0.001-5.38) | 0.696 | (0.014-2.85) |
| trans-p-Menthane-1,7,8-triol 8- | 0.003 | (0.000-0.014) | 0.001 | (0.000-0.005) | 0.007 | (0.001-0.025) | 0.011 | (0.001-0.026) |
| glucoside | | | | | | | | |
| Uric acid | 2.257 | (0.576-4.54) | 2.042 | (0.967-6.36) | 2.795 | (0.892-4.56) | 2.631 | (1.40-4.58) |
| Uridine | 0.058 | (0.022-0.119) | 0.052 | (0.022-0.098) | 0.114 | (0.052-0.297) | 0.094 | (0.013-0.163) |
| Vitamin K1 2,3-epoxide | 0.218 | (0.002-0.814) | 0.293 | (0.042-0.809) | 0.071 | (0.001-0.286) | 0.103 | (0.011-0.257) |
| Xanthine | 0.141 | (0.023-0.478) | 0.115 | (0.045-0.250) | 0.049 | (0.016-0.100) | 0.054 | (0.014-0.111) |

TABLE 4
Three-fold cross validation results and mean AUC across the 3 folds for
AFP and for selected lipids and metabolites and their combination with
AFP. ROC curve AUCs for all samples are also shown as AUC (ALL).

| | K Fold 1 | K Fold 2 | K Fold 3 | Mean AUC | AUC (ALL) |
|---|---|---|---|---|---|
| AFP | 0.93 | 0.67 | 0.91 | 0.83 | 0.80 |
| SPH | 0.97 | 0.92 | 0.96 | 0.95 | 0.94 |
| ST | 0.83 | 0.94 | 0.88 | 0.88 | 0.89 |
| LysoPS | 0.85 | 0.81 | 0.94 | 0.87 | 0.86 |
| PC(18:1_24:2) | 0.97 | 0.91 | 0.88 | 0.92 | 0.91 |
| ST(d18:1_20:2) | 0.83 | 0.94 | 0.88 | 0.88 | 0.89 |
| PE(20:0p_20:3) | 0.87 | 1 | 0.69 | 0.85 | 0.88 |
| SPH + AFP | 0.98 | 0.88 | 0.98 | 0.95 | 0.95 |
| ST + AFP | 0.97 | 0.96 | 0.98 | 0.97 | 0.96 |
| LysoPS + AFP | 0.96 | 0.75 | 1 | 0.90 | 0.93 |
| PC(18:1_24:2) + AFP | 0.99 | 0.89 | 0.99 | 0.96 | 0.96 |
| ST(d18:1_20:2) + AFP | 0.97 | 0.96 | 0.98 | 0.97 | 0.96 |
| PE(20:0p_20:3) + AFP | 0.98 | 1 | 0.96 | 0.98 | 0.97 |
| LysoPC(18:2) + AFP | 0.91 | 0.81 | 0.85 | 0.86 | 0.89 |
| Cholesterol sulfate + AFP | 0.92 | 0.79 | 0.97 | 0.89 | 0.86 |
| Taurodeoxycholic acid + AFP | 0.91 | 0.74 | 0.93 | 0.86 | 0.84 |
| Glycocholic acid + AFP | 0.92 | 0.80 | 0.97 | 0.90 | 0.86 |

AFP: alpha-fetoprotein; LysoPS: lysophosphatidylserine; LysoPC: lysophosphatidylcholine; PC: phosphatidylcholine; PE: phosphatidylethanolamine; SPH: sphingosine; ST: sulfatide.

TABLE 5
Sensitivity and specificity for early HCC detection
(BCLA 0 and A) were calculated for AFP and for selected
lipids and metabolites in combination with AFP.

| | Sensitivity (%) | Specificity (%) |
|---|---|---|
| AFP at 20 ng/ml | 45 | 95 |
| SPH + AFP | 90 | 97 |
| ST + AFP | 95 | 90 |
| LysoPS + AFP | 95 | 77 |
| PC(18:1/24:2) + AFP | 100 | 87 |
| PE(20:0p/20:3) + AFP | 95 | 87 |
| Taurodeoxycholic acid + AFP | 80 | 74 |
| Glycocholic acid + AFP | 85 | 67 |
| Cholesterol sulfate + AFP | 60 | 90 |
| LysoPC(18:2) + AFP | 80 | 92 |

AFP: alpha fetoprotein; LysoPS: lysophosphatidylserine; LysoPC: lysophosphatidylcholine; PC: phosphatidylcholine; PE: phosphatidylethanolamine; SPH: sphingosine; ST: sulfatide.

TABLE 6
Demographic and clinical parameters in 72 participants
with cirrhosis (31 with HCC and 41 without HCC).

| | HCC (n = 31) | | non-HCC (n = 41) | | p value |
|---|---|---|---|---|---|
| Male | 20 | (65%) | 20 | (49%) | 0.183 |
| Age | 62.4 (48.9-77.9) - 63.7 | | 59 (42-74) - 58.5 | | 0.074 |
| BMI | 28.9 (20.1-46.6) - 27.5 | | 32.3 (19.7-51.6) - 31.6 | | 0.045 |
| Diabetes | 12 | (39%) | 14 | (34%) | 0.670 |
| Race | | | | | 0.035 |
| Black | 10 | (32%) | 10 | (24%) | |
| White | 9 | (29%) | 24 | (59%) | |
| Hispanic | 12 | (39%) | 6 | (15%) | |
| Unknown | 0 | (0%) | 1 | (2%) | |
| Etiology | | | | | 0.061 |
| ETOH | 7 | (23%) | 10 | (24%) | |
| HCV | 14 | (45%) | 18 | (44%) | |
| NAFLD | 4 | (13%) | 12 | (29%) | |
| HCV/ETOH | 6 | (19%) | 1 | (2%) | |
| Smoking | | | | | 0.290 |
| Never-smoker | 11 | (35%) | 20 | (49%) | |
| Active | 9 | (29%) | 13 | (32%) | |
| Former | 11 | (35%) | 8 | (20%) | |
| Alcohol Consumption | | | | | 0.075 |
| No alcohol consumption | 8 | (26%) | 21 | (51%) | |
| Active Social | 5 | (16%) | 8 | (20%) | |
| Active Heavy | 4 | (13%) | 4 | (10%) | |
| Former Heavy | 14 | (45%) | 8 | (20%) | |
| Encephalopathy | | | | | 0.886 |
| None | 22 | (71%) | 30 | (73%) | |
| Controlled/Mild | 8 | (26%) | 9 | (22%) | |
| Uncontrolled/Severe | 1 | (3%) | 2 | (5%) | |
| Ascites | | | | | 0.825 |
| None | 19 | (61%) | 28 | (68%) | |
| Controlled/Mild | 11 | (35%) | 12 | (29%) | |
| Uncontrolled/Severe | 1 | (3%) | 1 | (2%) | |
| Clinical Labs | | | | | |
| Creatinine (mg/dL) | 0.938 (0.58-2.16) - 0.83 | | 1.15 (0.53-7.44) - 0.86 | | 0.358 |
| Albumin (g/dL) | 3.5 (2.6-4.5) - 3.6 | | 3.88 (2.4-5) - 3.9 | | 0.008 |
| ALT (U/L) | 64.12 (18-228) - 43 | | 45.90 (5-209) - 32 | | 0.092 |
| AST (U/L) | 98.1 (27-686) - 63 | | 60.83 (9-184) - 48 | | 0.065 |
| Bilirubin (mg/dL) | 1.51 (0.3-7.5) - 0.9 | | 1.81 (0.2-19.2) - 0.9 | | 0.623 |
| INR | 1.22 (0.9-2.0) - 1.2 | | 1.16 (1-1.6) - 1.1 | | 0.198 |
| Platelets (k/uL) | 128.3 (34-372) - 109 | | 143.66 (42-326) - 136 | | 0.352 |
| AFP (ng/ml) | 178.1 (2-1948) - 24 | | 8.44 (1-74) - 5 | | 0.021 |
| Sodium (mmol/L) | 140 (134-144) - 140 | | 135.73 (12-146) - 139 | | 0.309 |
| MELD Score | 11 (6-22) - 9 | | 11 (6-25) - 9 | | 0.993 |
| Child Pugh Class | | | | | 0.607 |
| A | 18 | (58%) | 28 | (68%) | |
| B | 10 | (32%) | 9 | (22%) | |
| C | 3 | (10%) | 4 | (10%) | |

| BCLC Class | | |
|---|---|---|
| 0 | 2 | (6%) |
| A | 18 | (58%) |
| B | 5 | (16%) |
| C | 3 | (10%) |
| D | 3 | (10%) |

| | |
|---|---|
| Largest Tumor Diameter (cm) | 3.7 (1.1-13) - 3.1 |

Data are displayed as n (%) or mean (range) - median.
AFP: alpha-fetoprotein;
ALT: alanine transaminase;
AST: aspartate transaminase;
BMI: body mass index;
ETOH: alcohol;
HCV: hepatitis C;
INR: international normalized ratio;
NAFLD: nonalcoholic fatty liver disease

TABLE 7A
AUC scores and 95% confidence intervals (CI) for selected
lipid classes, lipid species, and metabolites.

| | AUC for Y | 95% CI | 95% CI |
|---|---|---|---|
| variable | (n = 72) | lower | upper |
| SPH | 0.9434 | 0.8863 | 1 |
| PC(18:1/24:2) | 0.9146 | 0.8563 | 0.9729 |
| ST | 0.8902 | 0.8261 | 0.9544 |
| ST(d18:1/20:2) | 0.8902 | 0.8261 | 0.9544 |
| PE(20:0p/20:3) | 0.8780 | 0.8115 | 0.9446 |
| LysoPS | 0.8607 | 0.7739 | 0.9476 |
| OAHFA | 0.7860 | 0.6863 | 0.8857 |
| Glycocholic acid | 0.7337 | 0.6182 | 0.8492 |
| Cholesterol sulfate | 0.7337 | 0.6179 | 0.8494 |
| Taurodeoxycholic acid | 0.7333 | 0.6179 | 0.8487 |
| PG | 0.7329 | 0.6208 | 0.8449 |
| CL | 0.7191 | 0.6123 | 0.8259 |
| Taurocholic acid | 0.7140 | 0.5944 | 0.8336 |
| LysoPC(18:2) | 0.7101 | 0.5883 | 0.8318 |
| Hex1Cer | 0.6947 | 0.5697 | 0.8197 |
| Hex2Cer | 0.6892 | 0.5621 | 0.8164 |
| DLCL | 0.6774 | 0.5918 | 0.7630 |
| FA | 0.6522 | 0.5416 | 0.7629 |
| CerP | 0.6255 | 0.5320 | 0.7190 |
| CerPE | 0.6200 | 0.5194 | 0.7206 |
| GD1a | 0.3950 | 0.3210 | 0.4690 |
| AcHexSiE | 0.3537 | 0.2832 | 0.4242 |

TABLE 7B
AUC scores and 95% confidence intervals (CI) for selected two-way
combinations of lipid classes, lipid species, and metabolites.

| | | AUC for Y | 95% CI | 95% CI |
|---|---|---|---|---|
| variable1 | variable2 | (n = 72) | lower | upper |
| SPH | PE(20:0p/20:3) | 0.9858 | 0.9682 | 1 |
| LysoPS | PE(20:0p/20:3) | 0.9843 | 0.9673 | 1 |
| SPH | PC(18:1/24:2) | 0.9843 | 0.9654 | 1 |
| SPH | ST | 0.9827 | 0.9625 | 1 |
| SPH | ST(d18:1/20:2) | 0.9827 | 0.9625 | 1 |
| LysoPS | PC(18:1/24:2) | 0.9819 | 0.9617 | 1 |
| ST | PC(18:1/24:2) | 0.9756 | 0.9422 | 1 |
| PC(18:1/24:2) | ST(d18:1/20:2) | 0.9756 | 0.9422 | 1 |
| LysoPS | ST | 0.9748 | 0.9490 | 1 |
| LysoPS | ST(d18:1/20:2) | 0.9748 | 0.9490 | 1 |
| Taurocholic acid | PC(18:1/24:2) | 0.9736 | 0.9460 | 1 |
| Cholesterol sulfate | PC(18:1/24:2) | 0.9732 | 0.9452 | 1 |
| Glycocholic acid | PC(18:1/24:2) | 0.9689 | 0.9371 | 1 |
| Taurodeoxycholic acid | PC(18:1/24:2) | 0.9677 | 0.9324 | 1 |
| CL | PC(18:1/24:2) | 0.9642 | 0.9355 | 0.9929 |
| ST | PE(20:0p/20:3) | 0.9634 | 0.9231 | 1 |
| PC(18:1/24:2) | PE(20:0p/20:3) | 0.9634 | 0.9231 | 1 |
| ST(d18:1/20:2) | PE(20:0p/20:3) | 0.9634 | 0.9231 | 1 |
| Taurodeoxycholic acid | ST | 0.9622 | 0.9236 | 1 |
| Taurodeoxycholic acid | ST(d18:1/20:2) | 0.9622 | 0.9236 | 1 |
| PG | ST | 0.9607 | 0.9291 | 0.9923 |
| PG | ST(d18:1/20:2) | 0.9607 | 0.9291 | 0.9923 |
| PG | PE(20:0p/20:3) | 0.9607 | 0.9310 | 0.9903 |
| LysoPC(18:2) | SPH | 0.9599 | 0.9210 | 0.9988 |
| PG | PC(18:1/24:2) | 0.9591 | 0.9192 | 0.9990 |
| OAHFA | PC(18:1/24:2) | 0.9583 | 0.9172 | 0.9994 |
| CL | SPH | 0.9575 | 0.9152 | 0.9998 |
| Hex2Cer | PC(18:1/24:2) | 0.9575 | 0.9129 | 1 |
| Taurocholic acid | ST | 0.9567 | 0.9160 | 0.9975 |
| Taurocholic acid | ST(d18:1/20:2) | 0.9567 | 0.9160 | 0.9975 |
| LysoPS | SPH | 0.9567 | 0.9147 | 0.9987 |
| Cholesterol sulfate | ST | 0.9559 | 0.9096 | 1 |
| Cholesterol sulfate | ST(d18:1/20:2) | 0.9559 | 0.9096 | 1 |
| FA | SPH | 0.9559 | 0.9130 | 0.9988 |
| AcHexSiE | SPH | 0.9552 | 0.9105 | 0.9998 |
| CL | ST | 0.9540 | 0.9208 | 0.9871 |
| CL | ST(d18:1/20:2) | 0.9540 | 0.9208 | 0.9871 |
| Glycocholic acid | ST | 0.9536 | 0.9060 | 1 |
| Glycocholic acid | ST(d18:1/20:2) | 0.9536 | 0.9060 | 1 |
| GD1a | SPH | 0.9520 | 0.9053 | 0.9988 |
| OAHFA | ST | 0.9512 | 0.9117 | 0.9908 |
| OAHFA | ST(d18:1/20:2) | 0.9512 | 0.9117 | 0.9908 |
| Cholesterol sulfate | SPH | 0.9500 | 0.8987 | 1 |
| Glycocholic acid | SPH | 0.9496 | 0.8984 | 1 |
| CL | PE(20:0p/20:3) | 0.9489 | 0.9136 | 0.9841 |
| Cholesterol sulfate | PE(20:0p/20:3) | 0.9481 | 0.9012 | 0.9949 |
| FA | PC(18:1/24:2) | 0.9477 | 0.8976 | 0.9977 |
| Glycocholic acid | PE(20:0p/20:3) | 0.9477 | 0.9004 | 0.9950 |
| PG | SPH | 0.9473 | 0.8899 | 1 |
| DLCL | SPH | 0.9457 | 0.8890 | 1 |
| CerPE | PC(18:1/24:2) | 0.9449 | 0.9046 | 0.9853 |
| DLCL | PC(18:1/24:2) | 0.9449 | 0.9046 | 0.9853 |
| Taurocholic acid | SPH | 0.9434 | 0.8846 | 1 |
| Taurodeoxycholic acid | SPH | 0.9426 | 0.8836 | 1 |
| CerPE | SPH | 0.9426 | 0.8820 | 1 |
| Hex1Cer | PC(18:1/24:2) | 0.9402 | 0.8879 | 0.9926 |
| CerP | SPH | 0.9394 | 0.8788 | 1 |
| AcHexSiE | PC(18:1/24:2) | 0.9390 | 0.8883 | 0.9897 |
| AcHexSiE | PE(20:0p/20:3) | 0.9390 | 0.8883 | 0.9897 |
| GD1a | PC(18:1/24:2) | 0.9371 | 0.8846 | 0.9895 |
| OAHFA | SPH | 0.9363 | 0.8722 | 1 |
| LysoPC(18:2) | ST | 0.9347 | 0.8751 | 0.9943 |
| LysoPC(18:2) | ST(d18:1/20:2) | 0.9347 | 0.8751 | 0.9943 |
| Taurocholic acid | PE(20:0p/20:3) | 0.9323 | 0.8717 | 0.9929 |
| LysoPC(18:2) | PC(18:1/24:2) | 0.9315 | 0.8654 | 0.9977 |
| CerP | PC(18:1/24:2) | 0.9315 | 0.8786 | 0.9845 |
| DLCL | ST | 0.9292 | 0.8837 | 0.9746 |
| DLCL | ST(d18:1/20:2) | 0.9292 | 0.8837 | 0.9746 |
| Hex1Cer | SPH | 0.9268 | 0.8518 | 1 |
| OAHFA | PE(20:0p/20:3) | 0.9268 | 0.8690 | 0.9847 |
| CerP | ST | 0.9256 | 0.8785 | 0.9728 |
| CerP | ST(d18:1/20:2) | 0.9256 | 0.8785 | 0.9728 |
| GD1a | PE(20:0p/20:3) | 0.9229 | 0.8660 | 0.9798 |
| DLCL | PE(20:0p/20:3) | 0.9213 | 0.8736 | 0.9691 |
| LysoPS | PG | 0.9205 | 0.8589 | 0.9822 |
| Hex2Cer | PE(20:0p/20:3) | 0.9178 | 0.8551 | 0.9805 |
| Hex2Cer | SPH | 0.9166 | 0.8320 | 1 |
| Hex2Cer | ST | 0.9150 | 0.8491 | 0.9810 |
| Hex2Cer | ST(d18:1/20:2) | 0.9150 | 0.8491 | 0.9810 |
| Taurodeoxycholic acid | PE(20:0p/20:3) | 0.9142 | 0.8424 | 0.9861 |
| CL | LysoPS | 0.9127 | 0.8470 | 0.9783 |
| Hex1Cer | ST | 0.9099 | 0.8417 | 0.9781 |
| Hex1Cer | ST(d18:1/20:2) | 0.9099 | 0.8417 | 0.9781 |
| DLCL | LysoPS | 0.9087 | 0.8379 | 0.9796 |
| FA | ST | 0.9079 | 0.8413 | 0.9746 |
| FA | ST(d18:1/20:2) | 0.9079 | 0.8413 | 0.9746 |
| AcHexSiE | ST | 0.9024 | 0.8410 | 0.9638 |
| AcHexSiE | ST(d18:1/20:2) | 0.9024 | 0.8410 | 0.9638 |
| GD1a | ST | 0.8985 | 0.8348 | 0.9623 |
| GD1a | ST(d18:1/20:2) | 0.8985 | 0.8348 | 0.9623 |
| AcHexSiE | LysoPS | 0.8969 | 0.8270 | 0.9668 |
| Hex1Cer | PE(20:0p/20:3) | 0.8958 | 0.8207 | 0.9708 |
| CerPE | PE(20:0p/20:3) | 0.8954 | 0.8259 | 0.9649 |
| FA | PE(20:0p/20:3) | 0.8926 | 0.8215 | 0.9637 |
| LysoPC(18:2) | PE(20:0p/20:3) | 0.8922 | 0.8156 | 0.9688 |
| CerPE | ST | 0.8914 | 0.8151 | 0.9677 |
| CerPE | ST(d18:1/20:2) | 0.8914 | 0.8151 | 0.9677 |
| ST | ST(d18:1/20:2) | 0.8902 | 0.8261 | 0.9544 |
| Taurocholic acid | LysoPS | 0.8883 | 0.8108 | 0.9658 |
| LysoPS | OAHFA | 0.8879 | 0.8083 | 0.9675 |
| CerPE | LysoPS | 0.8867 | 0.8079 | 0.9655 |
| Taurodeoxycholic acid | LysoPS | 0.8843 | 0.8049 | 0.9638 |
| LysoPC(18:2) | LysoPS | 0.8796 | 0.7911 | 0.9681 |
| GD1a | LysoPS | 0.8741 | 0.7949 | 0.9533 |
| CerP | LysoPS | 0.8706 | 0.7858 | 0.9553 |
| Hex2Cer | LysoPS | 0.8686 | 0.7715 | 0.9657 |
| FA | LysoPS | 0.8670 | 0.7755 | 0.9586 |
| LysoPC(18:2) | OAHFA | 0.8615 | 0.7756 | 0.9475 |
| AcHexSiE | OAHFA | 0.8615 | 0.7867 | 0.9364 |
| Hex1Cer | LysoPS | 0.8603 | 0.7622 | 0.9585 |
| CerP | PE(20:0p/20:3) | 0.8548 | 0.7680 | 0.9416 |
| Taurodeoxycholic acid | AcHexSiE | 0.8505 | 0.7646 | 0.9364 |
| FA | OAHFA | 0.8438 | 0.7586 | 0.9290 |
| CL | OAHFA | 0.8415 | 0.7485 | 0.9344 |
| Cholesterol sulfate | LysoPS | 0.8395 | 0.7359 | 0.9431 |
| Glycocholic acid | LysoPS | 0.8379 | 0.7328 | 0.9431 |
| Glycocholic acid | OAHFA | 0.8356 | 0.7420 | 0.9291 |
| Cholesterol sulfate | OAHFA | 0.8328 | 0.7382 | 0.9274 |
| GD1a | OAHFA | 0.8324 | 0.7479 | 0.9169 |
| LysoPC(18:2) | DLCL | 0.8320 | 0.7335 | 0.9305 |
| Taurocholic acid | AcHexSiE | 0.8281 | 0.7328 | 0.9234 |
| OAHFA | PG | 0.8261 | 0.7292 | 0.9230 |
| Cholesterol sulfate | AcHexSiE | 0.8249 | 0.7291 | 0.9208 |
| Glycocholic acid | AcHexSiE | 0.8245 | 0.7286 | 0.9205 |
| DLCL | OAHFA | 0.8202 | 0.7255 | 0.9149 |
| Taurodeoxycholic acid | OAHFA | 0.8167 | 0.7145 | 0.9189 |
| LysoPC(18:2) | AcHexSiE | 0.8155 | 0.7186 | 0.9124 |
| Taurocholic acid | OAHFA | 0.8131 | 0.7101 | 0.9162 |
| LysoPC(18:2) | PG | 0.8127 | 0.7081 | 0.9174 |
| LysoPC(18:2) | Hex2Cer | 0.8120 | 0.7104 | 0.9135 |
| Glycocholic acid | DLCL | 0.8116 | 0.7127 | 0.9104 |
| Glycocholic acid | CL | 0.8108 | 0.7107 | 0.9109 |
| Cholesterol sulfate | DLCL | 0.8108 | 0.7117 | 0.9099 |
| Cholesterol sulfate | CL | 0.8104 | 0.7106 | 0.9102 |
| Taurodeoxycholic acid | DLCL | 0.8045 | 0.7036 | 0.9053 |
| Taurodeoxycholic acid | CL | 0.8041 | 0.7007 | 0.9075 |
| CL | PG | 0.8033 | 0.7020 | 0.9046 |
| LysoPC(18:2) | CL | 0.8021 | 0.6978 | 0.9064 |
| AcHexSiE | CL | 0.8017 | 0.7135 | 0.8899 |
| DLCL | FA | 0.8009 | 0.7134 | 0.8885 |
| LysoPC(18:2) | Hex1Cer | 0.7986 | 0.6905 | 0.9066 |
| Taurodeoxycholic acid | PG | 0.7946 | 0.6880 | 0.9013 |
| Hex2Cer | OAHFA | 0.7943 | 0.6804 | 0.9081 |
| AcHexSiE | PG | 0.7915 | 0.6932 | 0.8898 |
| Taurocholic acid | CL | 0.7911 | 0.6846 | 0.8977 |
| DLCL | PG | 0.7907 | 0.6874 | 0.8940 |
| Taurocholic acid | DLCL | 0.7895 | 0.6846 | 0.8945 |
| CerPE | OAHFA | 0.7884 | 0.6851 | 0.8916 |
| CerP | OAHFA | 0.7876 | 0.6845 | 0.8907 |
| Hex1Cer | OAHFA | 0.7872 | 0.6714 | 0.9030 |
| Glycocholic acid | PG | 0.7836 | 0.6760 | 0.8913 |
| Cholesterol sulfate | PG | 0.7828 | 0.6751 | 0.8906 |
| AcHexSiE | Hex2Cer | 0.7828 | 0.6789 | 0.8868 |
| CL | GD1a | 0.7821 | 0.6852 | 0.8789 |
| Hex1Cer | PG | 0.7769 | 0.6655 | 0.8884 |
| FA | Hex1Cer | 0.7758 | 0.6669 | 0.8847 |
| FA | Hex2Cer | 0.7750 | 0.6640 | 0.8859 |
| AcHexSiE | Hex1Cer | 0.7734 | 0.6664 | 0.8804 |
| LysoPC(18:2) | GD1a | 0.7730 | 0.6634 | 0.8826 |
| AcHexSiE | DLCL | 0.7718 | 0.6961 | 0.8476 |
| LysoPC(18:2) | CerPE | 0.7714 | 0.6597 | 0.8832 |
| Glycocholic acid | CerPE | 0.7703 | 0.6584 | 0.8821 |
| Cholesterol sulfate | CerPE | 0.7691 | 0.6565 | 0.8817 |
| Taurocholic acid | PG | 0.7687 | 0.6557 | 0.8817 |
| AcHexSiE | FA | 0.7651 | 0.6669 | 0.8634 |
| FA | PG | 0.7628 | 0.6567 | 0.8688 |
| Hex2Cer | PG | 0.7596 | 0.6441 | 0.8752 |
| GD1a | PG | 0.7592 | 0.6514 | 0.8671 |
| Taurodeoxycholic acid | FA | 0.7569 | 0.6421 | 0.8717 |
| DLCL | Hex1Cer | 0.7565 | 0.6381 | 0.8749 |
| CerP | CL | 0.7561 | 0.6499 | 0.8622 |
| Taurodeoxycholic acid | CerPE | 0.7557 | 0.6423 | 0.8691 |
| Glycocholic acid | CerP | 0.7545 | 0.6423 | 0.8667 |
| Cholesterol sulfate | CerP | 0.7533 | 0.6409 | 0.8658 |
| Cholesterol sulfate | GD1a | 0.7533 | 0.6398 | 0.8669 |
| CL | FA | 0.7522 | 0.6411 | 0.8632 |
| Taurodeoxycholic acid | GD1a | 0.7514 | 0.6391 | 0.8637 |
| Taurodeoxycholic acid | Hex2Cer | 0.7514 | 0.6369 | 0.8658 |
| Cholesterol sulfate | FA | 0.7502 | 0.6341 | 0.8663 |
| Glycocholic acid | GD1a | 0.7498 | 0.6355 | 0.8641 |
| Glycocholic acid | FA | 0.7482 | 0.6320 | 0.8645 |
| CerP | PG | 0.7467 | 0.6346 | 0.8587 |
| FA | GD1a | 0.7447 | 0.6376 | 0.8518 |
| CL | DLCL | 0.7443 | 0.6393 | 0.8493 |
| LysoPC(18:2) | Taurodeoxycholic acid | 0.7396 | 0.6245 | 0.8546 |
| Taurodeoxycholic acid | CerP | 0.7392 | 0.6238 | 0.8545 |
| DLCL | GD1a | 0.7392 | 0.6515 | 0.8268 |
| Taurodeoxycholic acid | Hex1Cer | 0.7388 | 0.6195 | 0.8581 |
| CerPE | CL | 0.7364 | 0.6245 | 0.8484 |
| CL | Hex1Cer | 0.7360 | 0.6142 | 0.8579 |
| Taurodeoxycholic acid | Glycocholic acid | 0.7356 | 0.6209 | 0.8504 |
| Taurodeoxycholic acid | Cholesterol sulfate | 0.7349 | 0.6201 | 0.8497 |
| Taurodeoxycholic acid | Taurocholic acid | 0.7349 | 0.6198 | 0.8499 |
| Taurocholic acid | GD1a | 0.7345 | 0.6173 | 0.8516 |
| Glycocholic acid | Hex1Cer | 0.7341 | 0.6145 | 0.8536 |
| AcHexSiE | CerP | 0.7337 | 0.6468 | 0.8206 |
| DLCL | Hex2Cer | 0.7333 | 0.6118 | 0.8548 |
| Taurocholic acid | CerPE | 0.7321 | 0.6148 | 0.8494 |
| Cholesterol sulfate | Hex1Cer | 0.7301 | 0.6100 | 0.8503 |
| Glycocholic acid | Taurocholic acid | 0.7270 | 0.6095 | 0.8445 |
| Cholesterol sulfate | Taurocholic acid | 0.7262 | 0.6082 | 0.8442 |
| LysoPC(18:2) | Taurocholic acid | 0.7246 | 0.6059 | 0.8434 |
| LysoPC(18:2) | CerP | 0.7223 | 0.6029 | 0.8417 |
| Glycocholic acid | Hex2Cer | 0.7215 | 0.6022 | 0.8408 |
| CerPE | PG | 0.7199 | 0.6031 | 0.8367 |
| GD1a | Hex2Cer | 0.7199 | 0.6013 | 0.8385 |
| CerPE | Hex1Cer | 0.7191 | 0.5990 | 0.8392 |
| LysoPC(18:2) | Glycocholic acid | 0.7183 | 0.5976 | 0.8391 |
| LysoPC(18:2) | Cholesterol sulfate | 0.7183 | 0.5976 | 0.8390 |
| Cholesterol sulfate | Hex2Cer | 0.7175 | 0.5970 | 0.8381 |
| CerPE | DLCL | 0.7175 | 0.6148 | 0.8203 |
| GD1a | Hex1Cer | 0.7175 | 0.5987 | 0.8364 |
| AcHexSiE | CerPE | 0.7144 | 0.6170 | 0.8118 |
| Taurocholic acid | Hex2Cer | 0.7136 | 0.5927 | 0.8345 |
| Taurocholic acid | CerP | 0.7132 | 0.5932 | 0.8333 |
| Taurocholic acid | FA | 0.7113 | 0.5902 | 0.8323 |
| LysoPC(18:2) | FA | 0.7105 | 0.5887 | 0.8322 |
| Taurocholic acid | Hex1Cer | 0.7105 | 0.5872 | 0.8337 |
| CerPE | Hex2Cer | 0.7077 | 0.5848 | 0.8306 |
| CL | Hex2Cer | 0.7073 | 0.5792 | 0.8355 |
| CerP | DLCL | 0.7026 | 0.6048 | 0.8004 |
| CerP | GD1a | 0.7010 | 0.6058 | 0.7963 |
| CerP | Hex1Cer | 0.6975 | 0.5726 | 0.8224 |
| CerPE | FA | 0.6971 | 0.5844 | 0.8098 |
| CerP | FA | 0.6951 | 0.5811 | 0.8092 |
| Hex1Cer | Hex2Cer | 0.6916 | 0.5629 | 0.8203 |
| CerP | Hex2Cer | 0.6900 | 0.5613 | 0.8187 |
| CerPE | GD1a | 0.6837 | 0.5798 | 0.7876 |
| CerP | CerPE | 0.6707 | 0.5595 | 0.7819 |
| AcHexSiE | GD1a | 0.6703 | 0.5903 | 0.7504 |
| Glycocholic acid | Cholesterol sulfate | 0.6444 | 0.5124 | 0.7763 |

TABLE 7C
AUC scores and 95% confidence intervals (CI) for selected three-way
combinations of lipid classes, lipid species, and metabolites.

| | | | AUC for Y | 95% CI | 95% CI |
|---|---|---|---|---|---|
| variable1 | variable2 | variable3 | (n = 72) | lower | upper |
| CL | SPH | PE(20:0p/20:3) | 1 | 1 | 1 |
| LysoPS | PG | ST | 1 | 1 | 1 |
| LysoPS | PG | ST(d18:1/20:2) | 1 | 1 | 1 |
| LysoPS | PG | PE(20:0p/20:3) | 1 | 1 | 1 |
| CL | SPH | ST | 0.9992 | 0.9970 | 1 |
| CL | SPH | ST(d18:1/20:2) | 0.9992 | 0.9970 | 1 |
| CL | SPH | PC(18:1/24:2) | 0.9984 | 0.9947 | 1 |
| LysoPS | SPH | PE(20:0p/20:3) | 0.9961 | 0.9890 | 1 |
| LysoPS | ST | PE(20:0p/20:3) | 0.9953 | 0.9885 | 1 |
| LysoPS | PC(18:1/24:2) | PE(20:0p/20:3) | 0.9953 | 0.9885 | 1 |
| LysoPS | ST(d18:1/20:2) | PE(20:0p/20:3) | 0.9953 | 0.9885 | 1 |
| SPH | PC(18:1/24:2) | PE(20:0p/20:3) | 0.9953 | 0.9873 | 1 |
| CL | LysoPS | PC(18:1/24:2) | 0.9949 | 0.9862 | 1 |
| PG | SPH | PE(20:0p/20:3) | 0.9949 | 0.9844 | 1 |
| PG | SPH | ST | 0.9945 | 0.9832 | 1 |
| PG | SPH | ST(d18:1/20:2) | 0.9945 | 0.9832 | 1 |
| SPH | ST | PC(18:1/24:2) | 0.9945 | 0.9843 | 1 |
| SPH | PC(18:1/24:2) | ST(d18:1/20:2) | 0.9945 | 0.9843 | 1 |
| CL | LysoPS | ST | 0.9941 | 0.9841 | 1 |
| CL | LysoPS | ST(d18:1/20:2) | 0.9941 | 0.9841 | 1 |
| Taurocholic acid | LysoPS | PC(18:1/24:2) | 0.9937 | 0.9828 | 1 |
| SPH | ST | PE(20:0p/20:3) | 0.9937 | 0.9838 | 1 |
| SPH | ST(d18:1/20:2) | PE(20:0p/20:3) | 0.9937 | 0.9838 | 1 |
| Glycocholic acid | LysoPS | PE(20:0p/20:3) | 0.9929 | 0.9813 | 1 |
| GD1a | SPH | PE(20:0p/20:3) | 0.9929 | 0.9827 | 1 |
| AcHexSiE | LysoPS | PE(20:0p/20:3) | 0.9921 | 0.9823 | 1 |
| AcHexSiE | SPH | PE(20:0p/20:3) | 0.9921 | 0.9809 | 1 |
| CerPE | LysoPS | PC(18:1/24:2) | 0.9921 | 0.9806 | 1 |
| LysoPS | ST | PC(18:1/24:2) | 0.9921 | 0.9786 | 1 |
| LysoPS | PC(18:1/24:2) | ST(d18:1/20:2) | 0.9921 | 0.9786 | 1 |
| OAHFA | ST | PC(18:1/24:2) | 0.9921 | 0.9806 | 1 |
| OAHFA | PC(18:1/24:2) | ST(d18:1/20:2) | 0.9921 | 0.9806 | 1 |
| PG | ST | PC(18:1/24:2) | 0.9921 | 0.9806 | 1 |
| PG | PC(18:1/24:2) | ST(d18:1/20:2) | 0.9921 | 0.9806 | 1 |
| Taurodeoxycholic acid | LysoPS | PC(18:1/24:2) | 0.9913 | 0.9785 | 1 |
| Cholesterol sulfate | LysoPS | PE(20:0p/20:3) | 0.9913 | 0.9776 | 1 |
| Taurocholic acid | SPH | PC(18:1/24:2) | 0.9913 | 0.9778 | 1 |
| LysoPS | PG | PC(18:1/24:2) | 0.9913 | 0.9771 | 1 |
| LysoPC(18:2) | LysoPS | PE(20:0p/20:3) | 0.9906 | 0.9766 | 1 |
| Glycocholic acid | SPH | PE(20:0p/20:3) | 0.9906 | 0.9759 | 1 |
| Cholesterol sulfate | ST | PE(20:0p/20:3) | 0.9906 | 0.9750 | 1 |
| Cholesterol sulfate | ST(d18:1/20:2) | PE(20:0p/20:3) | 0.9906 | 0.9750 | 1 |
| Taurocholic acid | LysoPS | PE(20:0p/20:3) | 0.9906 | 0.9766 | 1 |
| GD1a | LysoPS | PE(20:0p/20:3) | 0.9906 | 0.9793 | 1 |
| Glycocholic acid | LysoPS | PC(18:1/24:2) | 0.9898 | 0.9754 | 1 |
| Glycocholic acid | SPH | ST | 0.9898 | 0.9751 | 1 |
| Glycocholic acid | SPH | ST(d18:1/20:2) | 0.9898 | 0.9751 | 1 |
| Glycocholic acid | ST | PE(20:0p/20:3) | 0.9898 | 0.9718 | 1 |
| Glycocholic acid | ST(d18:1/20:2) | PE(20:0p/20:3) | 0.9898 | 0.9718 | 1 |
| Cholesterol sulfate | LysoPS | PC(18:1/24:2) | 0.9898 | 0.9754 | 1 |
| Cholesterol sulfate | SPH | ST | 0.9898 | 0.9751 | 1 |
| Cholesterol sulfate | SPH | ST(d18:1/20:2) | 0.9898 | 0.9751 | 1 |
| Cholesterol sulfate | SPH | PE(20:0p/20:3) | 0.9898 | 0.9746 | 1 |
| Cholesterol sulfate | ST | PC(18:1/24:2) | 0.9898 | 0.9735 | 1 |
| Cholesterol sulfate | PC(18:1/24:2) | ST(d18:1/20:2) | 0.9898 | 0.9735 | 1 |
| CerPE | SPH | PC(18:1/24:2) | 0.9898 | 0.9757 | 1 |
| LysoPS | SPH | PC(18:1/24:2) | 0.9898 | 0.9757 | 1 |
| LysoPC(18:2) | SPH | ST | 0.9890 | 0.9741 | 1 |
| LysoPC(18:2) | SPH | ST(d18:1/20:2) | 0.9890 | 0.9741 | 1 |
| Taurodeoxycholic acid | LysoPS | PE(20:0p/20:3) | 0.9890 | 0.9734 | 1 |
| Cholesterol sulfate | PC(18:1/24:2) | PE(20:0p/20:3) | 0.9890 | 0.9720 | 1 |
| Taurocholic acid | ST | PE(20:0p/20:3) | 0.9890 | 0.9714 | 1 |
| Taurocholic acid | PC(18:1/24:2) | PE(20:0p/20:3) | 0.9890 | 0.9714 | 1 |
| Taurocholic acid | ST(d18:1/20:2) | PE(20:0p/20:3) | 0.9890 | 0.9714 | 1 |
| CerPE | LysoPS | PE(20:0p/20:3) | 0.9890 | 0.9741 | 1 |
| Hex2Cer | ST | PC(18:1/24:2) | 0.9890 | 0.9722 | 1 |
| Hex2Cer | PC(18:1/24:2) | ST(d18:1/20:2) | 0.9890 | 0.9722 | 1 |
| LysoPS | SPH | ST | 0.9890 | 0.9741 | 1 |
| LysoPS | SPH | ST(d18:1/20:2) | 0.9890 | 0.9741 | 1 |
| Taurocholic acid | CL | PC(18:1/24:2) | 0.9886 | 0.9730 | 1 |
| Taurodeoxycholic acid | LysoPS | ST | 0.9882 | 0.9723 | 1 |
| Taurodeoxycholic acid | LysoPS | ST(d18:1/20:2) | 0.9882 | 0.9723 | 1 |
| Taurodeoxycholic acid | SPH | PC(18:1/24:2) | 0.9882 | 0.9721 | 1 |
| Glycocholic acid | SPH | PC(18:1/24:2) | 0.9882 | 0.9726 | 1 |
| Taurocholic acid | ST | PC(18:1/24:2) | 0.9882 | 0.9699 | 1 |
| Taurocholic acid | PC(18:1/24:2) | ST(d18:1/20:2) | 0.9882 | 0.9699 | 1 |
| AcHexSiE | SPH | PC(18:1/24:2) | 0.9882 | 0.9724 | 1 |
| DLCL | LysoPS | PE(20:0p/20:3) | 0.9882 | 0.9738 | 1 |
| PG | ST | PE(20:0p/20:3) | 0.9882 | 0.9738 | 1 |
| PG | PC(18:1/24:2) | PE(20:0p/20:3) | 0.9882 | 0.9738 | 1 |
| PG | ST(d18:1/20:2) | PE(20:0p/20:3) | 0.9882 | 0.9738 | 1 |
| Glycocholic acid | PC(18:1/24:2) | PE(20:0p/20:3) | 0.9878 | 0.9686 | 1 |
| AcHexSiE | PC(18:1/24:2) | PE(20:0p/20:3) | 0.9878 | 0.9639 | 1 |
| DLCL | LysoPS | PC(18:1/24:2) | 0.9878 | 0.9726 | 1 |
| PG | SPH | PC(18:1/24:2) | 0.9878 | 0.9719 | 1 |
| ST | PC(18:1/24:2) | PE(20:0p/20:3) | 0.9878 | 0.9639 | 1 |
| PC(18:1/24:2) | ST(d18:1/20:2) | PE(20:0p/20:3) | 0.9878 | 0.9639 | 1 |
| Taurodeoxycholic acid | SPH | ST | 0.9874 | 0.9707 | 1 |
| Taurodeoxycholic acid | SPH | ST(d18:1/20:2) | 0.9874 | 0.9707 | 1 |
| Cholesterol sulfate | CL | PC(18:1/24:2) | 0.9874 | 0.9710 | 1 |
| Cholesterol sulfate | SPH | PC(18:1/24:2) | 0.9874 | 0.9710 | 1 |
| Taurocholic acid | SPH | ST | 0.9874 | 0.9704 | 1 |
| Taurocholic acid | SPH | ST(d18:1/20:2) | 0.9874 | 0.9704 | 1 |
| FA | LysoPS | PE(20:0p/20:3) | 0.9874 | 0.9708 | 1 |
| GD1a | PC(18:1/24:2) | PE(20:0p/20:3) | 0.9874 | 0.9627 | 1 |
| LysoPC(18:2) | SPH | PE(20:0p/20:3) | 0.9866 | 0.9697 | 1 |
| Taurodeoxycholic acid | CL | PC(18:1/24:2) | 0.9866 | 0.9690 | 1 |
| Glycocholic acid | ST | PC(18:1/24:2) | 0.9866 | 0.9663 | 1 |
| Glycocholic acid | PC(18:1/24:2) | ST(d18:1/20:2) | 0.9866 | 0.9663 | 1 |
| Taurocholic acid | LysoPS | ST | 0.9866 | 0.9694 | 1 |
| Taurocholic acid | LysoPS | ST(d18:1/20:2) | 0.9866 | 0.9694 | 1 |
| Taurocholic acid | SPH | PE(20:0p/20:3) | 0.9866 | 0.9689 | 1 |
| FA | SPH | PC(18:1/24:2) | 0.9866 | 0.9697 | 1 |
| GD1a | SPH | PC(18:1/24:2) | 0.9866 | 0.9689 | 1 |
| OAHFA | SPH | PE(20:0p/20:3) | 0.9866 | 0.9700 | 1 |
| Glycocholic acid | CL | PC(18:1/24:2) | 0.9862 | 0.9687 | 1 |
| Glycocholic acid | PG | PC(18:1/24:2) | 0.9858 | 0.9676 | 1 |
| Cholesterol sulfate | PG | PC(18:1/24:2) | 0.9858 | 0.9676 | 1 |
| CerP | SPH | PE(20:0p/20:3) | 0.9858 | 0.9684 | 1 |
| CerPE | SPH | PE(20:0p/20:3) | 0.9858 | 0.9684 | 1 |
| DLCL | SPH | PC(18:1/24:2) | 0.9858 | 0.9684 | 1 |
| DLCL | SPH | PE(20:0p/20:3) | 0.9858 | 0.9682 | 1 |
| FA | SPH | PE(20:0p/20:3) | 0.9858 | 0.9684 | 1 |
| Hex1Cer | SPH | PE(20:0p/20:3) | 0.9858 | 0.9680 | 1 |
| LysoPS | OAHFA | PE(20:0p/20:3) | 0.9854 | 0.9679 | 1 |
| Taurodeoxycholic acid | CL | ST | 0.9851 | 0.9662 | 1 |
| Taurodeoxycholic acid | CL | ST(d18:1/20:2) | 0.9851 | 0.9662 | 1 |
| Taurodeoxycholic acid | SPH | PE(20:0p/20:3) | 0.9851 | 0.9659 | 1 |
| Glycocholic acid | LysoPS | ST | 0.9851 | 0.9667 | 1 |
| Glycocholic acid | LysoPS | ST(d18:1/20:2) | 0.9851 | 0.9667 | 1 |
| Cholesterol sulfate | LysoPS | ST | 0.9851 | 0.9666 | 1 |
| Cholesterol sulfate | LysoPS | ST(d18:1/20:2) | 0.9851 | 0.9666 | 1 |
| AcHexSiE | LysoPS | PC(18:1/24:2) | 0.9851 | 0.9668 | 1 |
| GD1a | SPH | ST | 0.9851 | 0.9670 | 1 |
| GD1a | SPH | ST(d18:1/20:2) | 0.9851 | 0.9670 | 1 |
| Hex2Cer | PC(18:1/24:2) | PE(20:0p/20:3) | 0.9851 | 0.9654 | 1 |
| CL | ST | PE(20:0p/20:3) | 0.9847 | 0.9665 | 1 |
| CL | PC(18:1/24:2) | PE(20:0p/20:3) | 0.9847 | 0.9665 | 1 |
| CL | ST(d18:1/20:2) | PE(20:0p/20:3) | 0.9847 | 0.9665 | 1 |
| Taurodeoxycholic acid | PG | ST | 0.9843 | 0.9651 | 1 |
| Taurodeoxycholic acid | PG | ST(d18:1/20:2) | 0.9843 | 0.9651 | 1 |
| Glycocholic acid | CerPE | PC(18:1/24:2) | 0.9843 | 0.9649 | 1 |
| Glycocholic acid | PG | PE(20:0p/20:3) | 0.9843 | 0.9644 | 1 |
| Cholesterol sulfate | AcHexSiE | PE(20:0p/20:3) | 0.9843 | 0.9633 | 1 |
| Cholesterol sulfate | CerPE | PC(18:1/24:2) | 0.9843 | 0.9649 | 1 |
| Cholesterol sulfate | Hex2Cer | PC(18:1/24:2) | 0.9843 | 0.9643 | 1 |
| Cholesterol sulfate | PG | PE(20:0p/20:3) | 0.9843 | 0.9644 | 1 |
| CerP | SPH | ST | 0.9843 | 0.9652 | 1 |
| CerP | SPH | ST(d18:1/20:2) | 0.9843 | 0.9652 | 1 |
| CerPE | SPH | ST | 0.9843 | 0.9654 | 1 |
| CerPE | SPH | ST(d18:1/20:2) | 0.9843 | 0.9654 | 1 |
| CerPE | ST | PC(18:1/24:2) | 0.9843 | 0.9623 | 1 |
| CerPE | PC(18:1/24:2) | ST(d18:1/20:2) | 0.9843 | 0.9623 | 1 |
| DLCL | ST | PC(18:1/24:2) | 0.9843 | 0.9623 | 1 |
| DLCL | PC(18:1/24:2) | ST(d18:1/20:2) | 0.9843 | 0.9623 | 1 |
| FA | SPH | ST | 0.9843 | 0.9654 | 1 |
| FA | SPH | ST(d18:1/20:2) | 0.9843 | 0.9654 | 1 |
| GD1a | LysoPS | PC(18:1/24:2) | 0.9843 | 0.9650 | 1 |
| LysoPS | OAHFA | PC(18:1/24:2) | 0.9843 | 0.9657 | 1 |
| DLCL | LysoPS | ST | 0.9839 | 0.9653 | 1 |
| DLCL | LysoPS | ST(d18:1/20:2) | 0.9839 | 0.9653 | 1 |
| Taurodeoxycholic acid | PC(18:1/24:2) | PE(20:0p/20:3) | 0.9835 | 0.9549 | 1 |
| Glycocholic acid | AcHexSiE | PE(20:0p/20:3) | 0.9835 | 0.9609 | 1 |
| Glycocholic acid | Hex2Cer | PC(18:1/24:2) | 0.9835 | 0.9627 | 1 |
| Taurocholic acid | AcHexSiE | PE(20:0p/20:3) | 0.9835 | 0.9619 | 1 |
| Taurocholic acid | FA | PC(18:1/24:2) | 0.9835 | 0.9615 | 1 |
| Taurocholic acid | PG | ST | 0.9835 | 0.9631 | 1 |
| Taurocholic acid | PG | PC(18:1/24:2) | 0.9835 | 0.9634 | 1 |
| Taurocholic acid | PG | ST(d18:1/20:2) | 0.9835 | 0.9631 | 1 |
| AcHexSiE | SPH | ST | 0.9835 | 0.9640 | 1 |
| AcHexSiE | SPH | ST(d18:1/20:2) | 0.9835 | 0.9640 | 1 |
| CerP | ST | PC(18:1/24:2) | 0.9835 | 0.9605 | 1 |
| CerP | PC(18:1/24:2) | ST(d18:1/20:2) | 0.9835 | 0.9605 | 1 |
| DLCL | SPH | ST | 0.9835 | 0.9639 | 1 |
| DLCL | SPH | ST(d18:1/20:2) | 0.9835 | 0.9639 | 1 |
| Hex1Cer | SPH | ST | 0.9835 | 0.9636 | 1 |
| Hex1Cer | SPH | ST(d18:1/20:2) | 0.9835 | 0.9636 | 1 |
| Hex2Cer | LysoPS | PC(18:1/24:2) | 0.9835 | 0.9631 | 1 |
| Hex2Cer | SPH | PC(18:1/24:2) | 0.9835 | 0.9636 | 1 |
| LysoPC(18:2) | PG | ST | 0.9827 | 0.9613 | 1 |
| LysoPC(18:2) | PG | ST(d18:1/20:2) | 0.9827 | 0.9613 | 1 |
| Taurodeoxycholic acid | ST | PE(20:0p/20:3) | 0.9827 | 0.9538 | 1 |
| Taurodeoxycholic acid | ST(d18:1/20:2) | PE(20:0p/20:3) | 0.9827 | 0.9538 | 1 |
| Cholesterol sulfate | OAHFA | PC(18:1/24:2) | 0.9827 | 0.9623 | 1 |
| CerP | LysoPS | PC(18:1/24:2) | 0.9827 | 0.9626 | 1 |
| CerP | SPH | PC(18:1/24:2) | 0.9827 | 0.9625 | 1 |
| Hex1Cer | SPH | PC(18:1/24:2) | 0.9827 | 0.9623 | 1 |
| OAHFA | SPH | PC(18:1/24:2) | 0.9827 | 0.9625 | 1 |
| OAHFA | ST | PE(20:0p/20:3) | 0.9827 | 0.9601 | 1 |
| OAHFA | ST(d18:1/20:2) | PE(20:0p/20:3) | 0.9827 | 0.9601 | 1 |
| SPH | ST | ST(d18:1/20:2) | 0.9827 | 0.9625 | 1 |
| Taurocholic acid | CerPE | PC(18:1/24:2) | 0.9823 | 0.9614 | 1 |
| CerP | LysoPS | PE(20:0p/20:3) | 0.9823 | 0.9619 | 1 |
| Taurodeoxycholic acid | ST | PC(18:1/24:2) | 0.9819 | 0.9526 | 1 |
| Taurodeoxycholic acid | PC(18:1/24:2) | ST(d18:1/20:2) | 0.9819 | 0.9526 | 1 |
| Glycocholic acid | OAHFA | PC(18:1/24:2) | 0.9819 | 0.9609 | 1 |
| CL | Hex2Cer | PC(18:1/24:2) | 0.9819 | 0.9587 | 1 |
| OAHFA | SPH | ST | 0.9819 | 0.9608 | 1 |
| OAHFA | SPH | ST(d18:1/20:2) | 0.9819 | 0.9608 | 1 |
| Taurodeoxycholic acid | CerPE | PC(18:1/24:2) | 0.9811 | 0.9586 | 1 |
| CL | OAHFA | PC(18:1/24:2) | 0.9811 | 0.9607 | 1 |
| FA | LysoPS | PC(18:1/24:2) | 0.9811 | 0.9594 | 1 |
| LysoPS | OAHFA | ST | 0.9811 | 0.9598 | 1 |
| LysoPS | OAHFA | ST(d18:1/20:2) | 0.9811 | 0.9598 | 1 |
| Taurocholic acid | OAHFA | PC(18:1/24:2) | 0.9807 | 0.9584 | 1 |
| CerP | LysoPS | ST | 0.9807 | 0.9594 | 1 |
| CerP | LysoPS | ST(d18:1/20:2) | 0.9807 | 0.9594 | 1 |
| CL | FA | PC(18:1/24:2) | 0.9807 | 0.9589 | 1 |
| LysoPC(18:2) | SPH | PC(18:1/24:2) | 0.9803 | 0.9580 | 1 |
| Cholesterol sulfate | PG | ST | 0.9803 | 0.9555 | 1 |
| Cholesterol sulfate | PG | ST(d18:1/20:2) | 0.9803 | 0.9555 | 1 |
| AcHexSiE | OAHFA | PC(18:1/24:2) | 0.9803 | 0.9611 | 0.9996 |
| AcHexSiE | PG | PE(20:0p/20:3) | 0.9803 | 0.9611 | 0.9996 |
| FA | ST | PC(18:1/24:2) | 0.9803 | 0.9532 | 1 |
| FA | PC(18:1/24:2) | ST(d18:1/20:2) | 0.9803 | 0.9532 | 1 |
| LysoPC(18:2) | LysoPS | PC(18:1/24:2) | 0.9795 | 0.9564 | 1 |
| Glycocholic acid | Cholesterol sulfate | PC(18:1/24:2) | 0.9795 | 0.9564 | 1 |
| Cholesterol sulfate | CL | ST | 0.9795 | 0.9550 | 1 |
| Cholesterol sulfate | CL | ST(d18:1/20:2) | 0.9795 | 0.9550 | 1 |
| Taurocholic acid | Hex2Cer | PC(18:1/24:2) | 0.9795 | 0.9555 | 1 |
| Hex2Cer | LysoPS | PE(20:0p/20:3) | 0.9795 | 0.9541 | 1 |
| Hex2Cer | SPH | PE(20:0p/20:3) | 0.9795 | 0.9551 | 1 |
| OAHFA | PG | ST | 0.9795 | 0.9574 | 1 |
| OAHFA | PG | ST(d18:1/20:2) | 0.9795 | 0.9574 | 1 |
| Hex2Cer | PG | PE(20:0p/20:3) | 0.9792 | 0.9559 | 1 |
| Glycocholic acid | PG | ST | 0.9788 | 0.9528 | 1 |
| Glycocholic acid | PG | ST(d18:1/20:2) | 0.9788 | 0.9528 | 1 |
| Hex1Cer | LysoPS | PC(18:1/24:2) | 0.9788 | 0.9542 | 1 |
| Hex1Cer | LysoPS | PE(20:0p/20:3) | 0.9788 | 0.9524 | 1 |
| Hex1Cer | ST | PC(18:1/24:2) | 0.9788 | 0.9472 | 1 |
| Hex1Cer | PC(18:1/24:2) | ST(d18:1/20:2) | 0.9788 | 0.9472 | 1 |
| Hex2Cer | SPH | ST | 0.9788 | 0.9547 | 1 |
| Hex2Cer | SPH | ST(d18:1/20:2) | 0.9788 | 0.9547 | 1 |
| Taurodeoxycholic acid | FA | PC(18:1/24:2) | 0.9780 | 0.9462 | 1 |
| Glycocholic acid | CL | ST | 0.9780 | 0.9524 | 1 |
| Glycocholic acid | CL | ST(d18:1/20:2) | 0.9780 | 0.9524 | 1 |
| Cholesterol sulfate | CL | PE(20:0p/20:3) | 0.9780 | 0.9539 | 1 |
| Cholesterol sulfate | Hex1Cer | PC(18:1/24:2) | 0.9780 | 0.9534 | 1 |
| CL | PG | PC(18:1/24:2) | 0.9780 | 0.9553 | 1 |
| LysoPC(18:2) | ST | PC(18:1/24:2) | 0.9772 | 0.9367 | 1 |
| LysoPC(18:2) | PC(18:1/24:2) | ST(d18:1/20:2) | 0.9772 | 0.9367 | 1 |
| Cholesterol sulfate | Taurocholic acid | PC(18:1/24:2) | 0.9772 | 0.9521 | 1 |
| Cholesterol sulfate | AcHexSiE | PC(18:1/24:2) | 0.9772 | 0.9515 | 1 |
| Cholesterol sulfate | DLCL | PC(18:1/24:2) | 0.9772 | 0.9522 | 1 |
| Cholesterol sulfate | FA | PC(18:1/24:2) | 0.9772 | 0.9521 | 1 |
| Glycocholic acid | CL | PE(20:0p/20:3) | 0.9768 | 0.9517 | 1 |
| Taurocholic acid | AcHexSiE | PC(18:1/24:2) | 0.9768 | 0.9511 | 1 |
| Taurocholic acid | DLCL | PC(18:1/24:2) | 0.9768 | 0.9518 | 1 |
| Taurocholic acid | GD1a | PC(18:1/24:2) | 0.9768 | 0.9511 | 1 |
| Taurodeoxycholic acid | Hex2Cer | PC(18:1/24:2) | 0.9764 | 0.9483 | 1 |
| Taurodeoxycholic acid | PG | PC(18:1/24:2) | 0.9764 | 0.9497 | 1 |
| Taurocholic acid | PG | PE(20:0p/20:3) | 0.9764 | 0.9503 | 1 |
| AcHexSiE | LysoPS | ST | 0.9764 | 0.9514 | 1 |
| AcHexSiE | LysoPS | ST(d18:1/20:2) | 0.9764 | 0.9514 | 1 |
| CerPE | PC(18:1/24:2) | PE(20:0p/20:3) | 0.9764 | 0.9496 | 1 |
| CL | PG | PE(20:0p/20:3) | 0.9764 | 0.9549 | 0.9979 |
| DLCL | ST | PE(20:0p/20:3) | 0.9764 | 0.9496 | 1 |
| DLCL | PC(18:1/24:2) | PE(20:0p/20:3) | 0.9764 | 0.9496 | 1 |
| DLCL | ST(d18:1/20:2) | PE(20:0p/20:3) | 0.9764 | 0.9496 | 1 |
| GD1a | PG | PE(20:0p/20:3) | 0.9764 | 0.9549 | 0.9979 |
| GD1a | ST | PC(18:1/24:2) | 0.9764 | 0.9441 | 1 |
| GD1a | PC(18:1/24:2) | ST(d18:1/20:2) | 0.9764 | 0.9441 | 1 |
| OAHFA | PG | PE(20:0p/20:3) | 0.9764 | 0.9520 | 1 |
| Glycocholic acid | DLCL | PC(18:1/24:2) | 0.9760 | 0.9502 | 1 |
| CL | OAHFA | ST | 0.9760 | 0.9522 | 0.9999 |
| CL | OAHFA | ST(d18:1/20:2) | 0.9760 | 0.9522 | 0.9999 |
| LysoPC(18:2) | LysoPS | ST | 0.9756 | 0.9495 | 1 |
| LysoPC(18:2) | LysoPS | ST(d18:1/20:2) | 0.9756 | 0.9495 | 1 |
| Glycocholic acid | Taurocholic acid | PC(18:1/24:2) | 0.9756 | 0.9494 | 1 |
| Cholesterol sulfate | GD1a | PC(18:1/24:2) | 0.9756 | 0.9483 | 1 |
| AcHexSiE | ST | PC(18:1/24:2) | 0.9756 | 0.9422 | 1 |
| AcHexSiE | PC(18:1/24:2) | ST(d18:1/20:2) | 0.9756 | 0.9422 | 1 |
| CerPE | LysoPS | ST | 0.9756 | 0.9490 | 1 |
| CerPE | LysoPS | ST(d18:1/20:2) | 0.9756 | 0.9490 | 1 |
| FA | LysoPS | ST | 0.9756 | 0.9485 | 1 |
| FA | LysoPS | ST(d18:1/20:2) | 0.9756 | 0.9485 | 1 |
| GD1a | LysoPS | ST | 0.9756 | 0.9498 | 1 |
| GD1a | LysoPS | ST(d18:1/20:2) | 0.9756 | 0.9498 | 1 |
| GD1a | OAHFA | PC(18:1/24:2) | 0.9756 | 0.9522 | 0.9991 |
| ST | PC(18:1/24:2) | ST(d18:1/20:2) | 0.9756 | 0.9422 | 1 |
| CerP | ST | PE(20:0p/20:3) | 0.9752 | 0.9472 | 1 |
| CerP | PC(18:1/24:2) | PE(20:0p/20:3) | 0.9752 | 0.9472 | 1 |
| CerP | ST(d18:1/20:2) | PE(20:0p/20:3) | 0.9752 | 0.9472 | 1 |
| LysoPC(18:2) | PC(18:1/24:2) | PE(20:0p/20:3) | 0.9748 | 0.9381 | 1 |
| AcHexSiE | OAHFA | PE(20:0p/20:3) | 0.9748 | 0.9488 | 1 |
| CL | Hex1Cer | PC(18:1/24:2) | 0.9748 | 0.9482 | 1 |
| LysoPS | ST | ST(d18:1/20:2) | 0.9748 | 0.9490 | 1 |
| Taurocholic acid | CerP | PC(18:1/24:2) | 0.9744 | 0.9475 | 1 |
| AcHexSiE | CL | PC(18:1/24:2) | 0.9744 | 0.9506 | 0.9983 |
| AcHexSiE | CL | PE(20:0p/20:3) | 0.9744 | 0.9506 | 0.9983 |
| Taurodeoxycholic acid | Cholesterol sulfate | PC(18:1/24:2) | 0.9740 | 0.9462 | 1 |
| Taurodeoxycholic acid | DLCL | PC(18:1/24:2) | 0.9740 | 0.9455 | 1 |
| Glycocholic acid | FA | PC(18:1/24:2) | 0.9740 | 0.9460 | 1 |
| Cholesterol sulfate | CerP | PC(18:1/24:2) | 0.9740 | 0.9462 | 1 |
| Taurocholic acid | Hex1Cer | PC(18:1/24:2) | 0.9740 | 0.9464 | 1 |
| GD1a | ST | PE(20:0p/20:3) | 0.9740 | 0.9395 | 1 |
| GD1a | ST(d18:1/20:2) | PE(20:0p/20:3) | 0.9740 | 0.9395 | 1 |
| OAHFA | PC(18:1/24:2) | PE(20:0p/20:3) | 0.9740 | 0.9367 | 1 |
| Taurodeoxycholic acid | Glycocholic acid | PC(18:1/24:2) | 0.9732 | 0.9442 | 1 |
| Taurodeoxycholic acid | Taurocholic acid | PC(18:1/24:2) | 0.9732 | 0.9453 | 1 |
| Taurodeoxycholic acid | AcHexSiE | PE(20:0p/20:3) | 0.9732 | 0.9386 | 1 |
| Hex2Cer | ST | PE(20:0p/20:3) | 0.9732 | 0.9352 | 1 |
| Hex2Cer | ST(d18:1/20:2) | PE(20:0p/20:3) | 0.9732 | 0.9352 | 1 |
| Hex1Cer | PG | PE(20:0p/20:3) | 0.9729 | 0.9447 | 1 |
| LysoPC(18:2) | Taurocholic acid | PC(18:1/24:2) | 0.9725 | 0.9434 | 1 |
| Taurodeoxycholic acid | PG | PE(20:0p/20:3) | 0.9725 | 0.9440 | 1 |
| Glycocholic acid | AcHexSiE | PC(18:1/24:2) | 0.9725 | 0.9424 | 1 |
| Cholesterol sulfate | OAHFA | ST | 0.9725 | 0.9419 | 1 |
| Cholesterol sulfate | OAHFA | ST(d18:1/20:2) | 0.9725 | 0.9419 | 1 |
| CerPE | Hex2Cer | PC(18:1/24:2) | 0.9725 | 0.9412 | 1 |
| CL | GD1a | PC(18:1/24:2) | 0.9725 | 0.9465 | 0.9984 |
| Hex2Cer | LysoPS | ST | 0.9725 | 0.9433 | 1 |
| Hex2Cer | LysoPS | ST(d18:1/20:2) | 0.9725 | 0.9433 | 1 |
| LysoPC(18:2) | Cholesterol sulfate | PC(18:1/24:2) | 0.9717 | 0.9424 | 1 |
| LysoPC(18:2) | PG | SPH | 0.9717 | 0.9416 | 1 |
| Taurodeoxycholic acid | AcHexSiE | ST | 0.9717 | 0.9384 | 1 |
| Taurodeoxycholic acid | AcHexSiE | ST(d18:1/20:2) | 0.9717 | 0.9384 | 1 |
| Taurodeoxycholic acid | DLCL | ST | 0.9717 | 0.9417 | 1 |
| Taurodeoxycholic acid | DLCL | ST(d18:1/20:2) | 0.9717 | 0.9417 | 1 |
| Taurodeoxycholic acid | OAHFA | ST | 0.9717 | 0.9427 | 1 |
| Taurodeoxycholic acid | OAHFA | ST(d18:1/20:2) | 0.9717 | 0.9427 | 1 |
| Glycocholic acid | CerP | PC(18:1/24:2) | 0.9717 | 0.9414 | 1 |
| Hex1Cer | PG | ST | 0.9713 | 0.9419 | 1 |
| Hex1Cer | PG | ST(d18:1/20:2) | 0.9713 | 0.9419 | 1 |
| LysoPC(18:2) | PG | PE(20:0p/20:3) | 0.9709 | 0.9415 | 1 |
| Taurodeoxycholic acid | CerP | ST | 0.9709 | 0.9399 | 1 |
| Taurodeoxycholic acid | CerP | ST(d18:1/20:2) | 0.9709 | 0.9399 | 1 |
| Taurodeoxycholic acid | Hex1Cer | PC(18:1/24:2) | 0.9709 | 0.9374 | 1 |
| Glycocholic acid | Hex1Cer | PC(18:1/24:2) | 0.9709 | 0.9404 | 1 |
| Glycocholic acid | OAHFA | ST | 0.9709 | 0.9395 | 1 |
| Glycocholic acid | OAHFA | ST(d18:1/20:2) | 0.9709 | 0.9395 | 1 |
| Taurocholic acid | OAHFA | ST | 0.9709 | 0.9409 | 1 |
| Taurocholic acid | OAHFA | ST(d18:1/20:2) | 0.9709 | 0.9409 | 1 |
| Hex2Cer | PG | ST | 0.9709 | 0.9411 | 1 |
| Hex2Cer | PG | ST(d18:1/20:2) | 0.9709 | 0.9411 | 1 |
| DLCL | FA | PC(18:1/24:2) | 0.9705 | 0.9388 | 1 |
| FA | PG | ST | 0.9705 | 0.9426 | 0.9984 |
| FA | PG | ST(d18:1/20:2) | 0.9705 | 0.9426 | 0.9984 |
| LysoPC(18:2) | AcHexSiE | SPH | 0.9701 | 0.9397 | 1 |
| LysoPC(18:2) | CL | ST | 0.9701 | 0.9384 | 1 |
| LysoPC(18:2) | CL | ST(d18:1/20:2) | 0.9701 | 0.9384 | 1 |
| Taurodeoxycholic acid | AcHexSiE | PC(18:1/24:2) | 0.9701 | 0.9357 | 1 |
| Taurodeoxycholic acid | OAHFA | PC(18:1/24:2) | 0.9701 | 0.9379 | 1 |
| Glycocholic acid | GD1a | PC(18:1/24:2) | 0.9701 | 0.9378 | 1 |
| FA | PC(18:1/24:2) | PE(20:0p/20:3) | 0.9701 | 0.9301 | 1 |
| Hex1Cer | Hex2Cer | PC(18:1/24:2) | 0.9701 | 0.9280 | 1 |
| Hex1Cer | LysoPS | ST | 0.9701 | 0.9393 | 1 |
| Hex1Cer | LysoPS | ST(d18:1/20:2) | 0.9701 | 0.9393 | 1 |
| Hex2Cer | PG | PC(18:1/24:2) | 0.9701 | 0.9350 | 1 |
| OAHFA | PG | PC(18:1/24:2) | 0.9701 | 0.9368 | 1 |
| FA | PG | PE(20:0p/20:3) | 0.9697 | 0.9432 | 0.9962 |
| Hex1Cer | ST | PE(20:0p/20:3) | 0.9697 | 0.9279 | 1 |
| Hex1Cer | ST(d18:1/20:2) | PE(20:0p/20:3) | 0.9697 | 0.9279 | 1 |
| LysoPC(18:2) | Glycocholic acid | PC(18:1/24:2) | 0.9693 | 0.9380 | 1 |
| Taurodeoxycholic acid | GD1a | PC(18:1/24:2) | 0.9693 | 0.9338 | 1 |
| Cholesterol sulfate | CerP | ST | 0.9693 | 0.9350 | 1 |
| Cholesterol sulfate | CerP | ST(d18:1/20:2) | 0.9693 | 0.9350 | 1 |
| Taurocholic acid | CL | PE(20:0p/20:3) | 0.9693 | 0.9379 | 1 |
| FA | ST | PE(20:0p/20:3) | 0.9693 | 0.9292 | 1 |
| FA | ST(d18:1/20:2) | PE(20:0p/20:3) | 0.9693 | 0.9292 | 1 |
| CerPE | FA | PC(18:1/24:2) | 0.9689 | 0.9367 | 1 |
| CerPE | PG | ST | 0.9689 | 0.9401 | 0.9977 |
| CerPE | PG | ST(d18:1/20:2) | 0.9689 | 0.9401 | 0.9977 |
| LysoPC(18:2) | Taurodeoxycholic acid | PC(18:1/24:2) | 0.9685 | 0.9335 | 1 |
| LysoPC(18:2) | GD1a | SPH | 0.9685 | 0.9365 | 1 |
| LysoPC(18:2) | ST | PE(20:0p/20:3) | 0.9685 | 0.9242 | 1 |
| LysoPC(18:2) | ST(d18:1/20:2) | PE(20:0p/20:3) | 0.9685 | 0.9242 | 1 |
| CerP | PG | ST | 0.9685 | 0.9414 | 0.9957 |
| CerP | PG | ST(d18:1/20:2) | 0.9685 | 0.9414 | 0.9957 |
| CerPE | OAHFA | PC(18:1/24:2) | 0.9685 | 0.9394 | 0.9977 |
| DLCL | PG | ST | 0.9685 | 0.9414 | 0.9957 |
| DLCL | PG | PC(18:1/24:2) | 0.9685 | 0.9374 | 0.9997 |
| DLCL | PG | ST(d18:1/20:2) | 0.9685 | 0.9414 | 0.9957 |
| DLCL | PG | PE(20:0p/20:3) | 0.9685 | 0.9428 | 0.9942 |
| FA | Hex2Cer | PC(18:1/24:2) | 0.9685 | 0.9353 | 1 |
| LysoPC(18:2) | CL | PC(18:1/24:2) | 0.9677 | 0.9329 | 1 |
| Glycocholic acid | CerP | ST | 0.9677 | 0.9326 | 1 |
| Glycocholic acid | CerP | ST(d18:1/20:2) | 0.9677 | 0.9326 | 1 |
| CerP | CL | PC(18:1/24:2) | 0.9677 | 0.9391 | 0.9964 |
| CL | OAHFA | PE(20:0p/20:3) | 0.9673 | 0.9370 | 0.9977 |
| LysoPC(18:2) | OAHFA | ST | 0.9670 | 0.9334 | 1 |
| LysoPC(18:2) | OAHFA | ST(d18:1/20:2) | 0.9670 | 0.9334 | 1 |
| Taurodeoxycholic acid | CerP | PC(18:1/24:2) | 0.9670 | 0.9310 | 1 |
| Taurocholic acid | AcHexSiE | ST | 0.9670 | 0.9323 | 1 |
| Taurocholic acid | AcHexSiE | ST(d18:1/20:2) | 0.9670 | 0.9323 | 1 |
| AcHexSiE | Hex2Cer | PC(18:1/24:2) | 0.9670 | 0.9257 | 1 |
| AcHexSiE | PG | PC(18:1/24:2) | 0.9670 | 0.9291 | 1 |
| CerPE | ST | PE(20:0p/20:3) | 0.9670 | 0.9215 | 1 |
| CerPE | ST(d18:1/20:2) | PE(20:0p/20:3) | 0.9670 | 0.9215 | 1 |
| DLCL | FA | SPH | 0.9670 | 0.9323 | 1 |
| DLCL | OAHFA | PC(18:1/24:2) | 0.9670 | 0.9358 | 0.9981 |
| FA | OAHFA | PC(18:1/24:2) | 0.9670 | 0.9251 | 1 |
| LysoPC(18:2) | Taurocholic acid | SPH | 0.9662 | 0.9301 | 1 |
| Cholesterol sulfate | DLCL | ST | 0.9662 | 0.9307 | 1 |
| Cholesterol sulfate | DLCL | ST(d18:1/20:2) | 0.9662 | 0.9307 | 1 |
| Taurocholic acid | DLCL | ST | 0.9662 | 0.9331 | 0.9992 |
| Taurocholic acid | DLCL | ST(d18:1/20:2) | 0.9662 | 0.9331 | 0.9992 |
| AcHexSiE | CL | SPH | 0.9662 | 0.9315 | 1 |
| LysoPC(18:2) | Glycocholic acid | SPH | 0.9654 | 0.9300 | 1 |
| LysoPC(18:2) | CerPE | SPH | 0.9654 | 0.9303 | 1 |
| Cholesterol sulfate | Hex2Cer | PE(20:0p/20:3) | 0.9654 | 0.9313 | 0.9995 |
| Taurocholic acid | CerP | ST | 0.9654 | 0.9318 | 0.9989 |
| Taurocholic acid | CerP | ST(d18:1/20:2) | 0.9654 | 0.9318 | 0.9989 |
| CerPE | PG | PC(18:1/24:2) | 0.9654 | 0.9321 | 0.9986 |
| CL | GD1a | PE(20:0p/20:3) | 0.9654 | 0.9358 | 0.9950 |
| DLCL | Hex2Cer | PC(18:1/24:2) | 0.9654 | 0.9283 | 1 |
| Hex1Cer | PG | PC(18:1/24:2) | 0.9654 | 0.9294 | 1 |
| FA | PG | PC(18:1/24:2) | 0.9650 | 0.9250 | 1 |
| LysoPC(18:2) | Taurodeoxycholic acid | SPH | 0.9646 | 0.9276 | 1 |
| LysoPC(18:2) | Cholesterol sulfate | SPH | 0.9646 | 0.9286 | 1 |
| LysoPC(18:2) | PG | PC(18:1/24:2) | 0.9646 | 0.9266 | 1 |
| Taurodeoxycholic acid | GD1a | ST | 0.9646 | 0.9261 | 1 |
| Taurodeoxycholic acid | GD1a | ST(d18:1/20:2) | 0.9646 | 0.9261 | 1 |
| Glycocholic acid | DLCL | ST | 0.9646 | 0.9283 | 1 |
| Glycocholic acid | DLCL | ST(d18:1/20:2) | 0.9646 | 0.9283 | 1 |
| AcHexSiE | PG | ST | 0.9646 | 0.9348 | 0.9944 |
| AcHexSiE | PG | ST(d18:1/20:2) | 0.9646 | 0.9348 | 0.9944 |
| CerP | CL | ST | 0.9646 | 0.9369 | 0.9923 |
| CerP | CL | ST(d18:1/20:2) | 0.9646 | 0.9369 | 0.9923 |
| CerPE | PG | PE(20:0p/20:3) | 0.9646 | 0.9352 | 0.9940 |
| CL | FA | ST | 0.9646 | 0.9336 | 0.9956 |
| CL | FA | ST(d18:1/20:2) | 0.9646 | 0.9336 | 0.9956 |
| GD1a | PG | ST | 0.9646 | 0.9348 | 0.9944 |
| GD1a | PG | ST(d18:1/20:2) | 0.9646 | 0.9348 | 0.9944 |
| CL | Hex2Cer | PE(20:0p/20:3) | 0.9642 | 0.9299 | 0.9985 |
| Taurodeoxycholic acid | AcHexSiE | SPH | 0.9638 | 0.9231 | 1 |
| GD1a | Hex2Cer | PC(18:1/24:2) | 0.9638 | 0.9213 | 1 |
| GD1a | PG | PC(18:1/24:2) | 0.9638 | 0.9245 | 1 |
| Hex1Cer | PC(18:1/24:2) | PE(20:0p/20:3) | 0.9638 | 0.9198 | 1 |
| Cholesterol sulfate | AcHexSiE | SPH | 0.9634 | 0.9257 | 1 |
| AcHexSiE | ST | PE(20:0p/20:3) | 0.9634 | 0.9231 | 1 |
| AcHexSiE | ST(d18:1/20:2) | PE(20:0p/20:3) | 0.9634 | 0.9231 | 1 |
| ST | ST(d18:1/20:2) | PE(20:0p/20:3) | 0.9634 | 0.9231 | 1 |
| LysoPC(18:2) | DLCL | ST | 0.9630 | 0.9247 | 1 |
| LysoPC(18:2) | DLCL | ST(d18:1/20:2) | 0.9630 | 0.9247 | 1 |
| LysoPC(18:2) | Hex2Cer | PC(18:1/24:2) | 0.9630 | 0.9267 | 0.9994 |
| Taurodeoxycholic acid | CL | PE(20:0p/20:3) | 0.9630 | 0.9270 | 0.9990 |
| Glycocholic acid | AcHexSiE | SPH | 0.9630 | 0.9250 | 1 |
| Cholesterol sulfate | AcHexSiE | ST | 0.9630 | 0.9194 | 1 |
| Cholesterol sulfate | AcHexSiE | ST(d18:1/20:2) | 0.9630 | 0.9194 | 1 |
| AcHexSiE | FA | PC(18:1/24:2) | 0.9630 | 0.9213 | 1 |
| CerPE | LysoPS | SPH | 0.9630 | 0.9224 | 1 |
| CL | GD1a | SPH | 0.9630 | 0.9268 | 0.9992 |
| Glycocholic acid | DLCL | PE(20:0p/20:3) | 0.9626 | 0.9273 | 0.9980 |
| LysoPC(18:2) | DLCL | SPH | 0.9622 | 0.9227 | 1 |
| LysoPC(18:2) | FA | SPH | 0.9622 | 0.9246 | 0.9999 |
| LysoPC(18:2) | OAHFA | PC(18:1/24:2) | 0.9622 | 0.9156 | 1 |
| Taurodeoxycholic acid | Glycocholic acid | ST | 0.9622 | 0.9232 | 1 |
| Taurodeoxycholic acid | Glycocholic acid | ST(d18:1/20:2) | 0.9622 | 0.9232 | 1 |
| Taurodeoxycholic acid | Cholesterol sulfate | ST | 0.9622 | 0.9242 | 1 |
| Taurodeoxycholic acid | Cholesterol sulfate | ST(d18:1/20:2) | 0.9622 | 0.9242 | 1 |
| Taurodeoxycholic acid | FA | ST | 0.9622 | 0.9225 | 1 |
| Taurodeoxycholic acid | FA | ST(d18:1/20:2) | 0.9622 | 0.9225 | 1 |
| Taurodeoxycholic acid | ST | ST(d18:1/20:2) | 0.9622 | 0.9236 | 1 |
| Cholesterol sulfate | DLCL | PE(20:0p/20:3) | 0.9622 | 0.9267 | 0.9978 |
| AcHexSiE | LysoPS | SPH | 0.9622 | 0.9242 | 1 |
| CL | Hex1Cer | ST | 0.9618 | 0.9265 | 0.9971 |
| CL | Hex1Cer | ST(d18:1/20:2) | 0.9618 | 0.9265 | 0.9971 |
| LysoPC(18:2) | Taurodeoxycholic acid | ST | 0.9614 | 0.9204 | 1 |
| LysoPC(18:2) | Taurodeoxycholic acid | ST(d18:1/20:2) | 0.9614 | 0.9204 | 1 |
| Glycocholic acid | Hex2Cer | PE(20:0p/20:3) | 0.9614 | 0.9248 | 0.9981 |
| DLCL | LysoPS | SPH | 0.9614 | 0.9209 | 1 |
| FA | GD1a | PC(18:1/24:2) | 0.9614 | 0.9193 | 1 |
| Hex2Cer | OAHFA | PC(18:1/24:2) | 0.9614 | 0.9213 | 1 |
| LysoPS | PG | SPH | 0.9614 | 0.9198 | 1 |
| LysoPC(18:2) | CerP | SPH | 0.9607 | 0.9222 | 0.9991 |
| LysoPC(18:2) | CL | SPH | 0.9607 | 0.9194 | 1 |
| Taurodeoxycholic acid | CerPE | ST | 0.9607 | 0.9214 | 1.0000 |
| Taurodeoxycholic acid | CerPE | ST(d18:1/20:2) | 0.9607 | 0.9214 | 1.0000 |
| Glycocholic acid | AcHexSiE | ST | 0.9607 | 0.9157 | 1 |
| Glycocholic acid | AcHexSiE | ST(d18:1/20:2) | 0.9607 | 0.9157 | 1 |
| Taurocholic acid | LysoPS | SPH | 0.9607 | 0.9218 | 0.9995 |
| AcHexSiE | CerPE | PC(18:1/24:2) | 0.9607 | 0.9263 | 0.9950 |
| AcHexSiE | DLCL | PC(18:1/24:2) | 0.9607 | 0.9263 | 0.9950 |
| AcHexSiE | DLCL | PE(20:0p/20:3) | 0.9607 | 0.9263 | 0.9950 |
| CL | Hex2Cer | ST | 0.9607 | 0.9239 | 0.9974 |
| CL | Hex2Cer | ST(d18:1/20:2) | 0.9607 | 0.9239 | 0.9974 |
| GD1a | LysoPS | SPH | 0.9607 | 0.9220 | 0.9993 |
| PG | ST | ST(d18:1/20:2) | 0.9607 | 0.9291 | 0.9923 |
| LysoPC(18:2) | CerPE | PC(18:1/24:2) | 0.9603 | 0.9196 | 1 |
| AcHexSiE | Hex2Cer | PE(20:0p/20:3) | 0.9603 | 0.9158 | 1 |
| LysoPC(18:2) | Taurocholic acid | ST | 0.9599 | 0.9188 | 1 |
| LysoPC(18:2) | Taurocholic acid | ST(d18:1/20:2) | 0.9599 | 0.9188 | 1 |
| LysoPC(18:2) | LysoPS | SPH | 0.9599 | 0.9171 | 1 |
| LysoPC(18:2) | OAHFA | SPH | 0.9599 | 0.9200 | 0.9998 |
| CerPE | CL | ST | 0.9599 | 0.9256 | 0.9941 |
| CerPE | CL | ST(d18:1/20:2) | 0.9599 | 0.9256 | 0.9941 |
| CerPE | Hex1Cer | PC(18:1/24:2) | 0.9599 | 0.9220 | 0.9977 |
| LysoPC(18:2) | AcHexSiE | PC(18:1/24:2) | 0.9591 | 0.9074 | 1 |
| Taurodeoxycholic acid | Taurocholic acid | ST | 0.9591 | 0.9196 | 0.9986 |
| Taurodeoxycholic acid | Taurocholic acid | ST(d18:1/20:2) | 0.9591 | 0.9196 | 0.9986 |
| Taurodeoxycholic acid | Hex2Cer | ST | 0.9591 | 0.9204 | 0.9978 |
| Taurodeoxycholic acid | Hex2Cer | ST(d18:1/20:2) | 0.9591 | 0.9204 | 0.9978 |
| Taurodeoxycholic acid | LysoPS | SPH | 0.9591 | 0.9176 | 1 |
| Cholesterol sulfate | Taurocholic acid | ST | 0.9591 | 0.9188 | 0.9994 |
| Cholesterol sulfate | Taurocholic acid | ST(d18:1/20:2) | 0.9591 | 0.9188 | 0.9994 |
| Cholesterol sulfate | GD1a | PE(20:0p/20:3) | 0.9591 | 0.9194 | 0.9988 |
| Cholesterol sulfate | Hex2Cer | ST | 0.9591 | 0.9157 | 1 |
| Cholesterol sulfate | Hex2Cer | ST(d18:1/20:2) | 0.9591 | 0.9157 | 1 |
| Taurocholic acid | AcHexSiE | SPH | 0.9591 | 0.9161 | 1 |
| Taurocholic acid | GD1a | ST | 0.9591 | 0.9186 | 0.9996 |
| Taurocholic acid | GD1a | ST(d18:1/20:2) | 0.9591 | 0.9186 | 0.9996 |
| AcHexSiE | CL | ST | 0.9591 | 0.9281 | 0.9901 |
| AcHexSiE | CL | ST(d18:1/20:2) | 0.9591 | 0.9281 | 0.9901 |
| AcHexSiE | Hex1Cer | PC(18:1/24:2) | 0.9591 | 0.9155 | 1 |
| CerP | PG | PC(18:1/24:2) | 0.9591 | 0.9191 | 0.9991 |
| CerPE | DLCL | PC(18:1/24:2) | 0.9587 | 0.9266 | 0.9908 |
| CerPE | GD1a | PC(18:1/24:2) | 0.9587 | 0.9224 | 0.9950 |
| DLCL | GD1a | PC(18:1/24:2) | 0.9587 | 0.9224 | 0.9950 |
| DLCL | OAHFA | ST | 0.9587 | 0.9243 | 0.9931 |
| DLCL | OAHFA | ST(d18:1/20:2) | 0.9587 | 0.9243 | 0.9931 |
| LysoPC(18:2) | CerP | ST | 0.9583 | 0.9165 | 1 |
| LysoPC(18:2) | CerP | ST(d18:1/20:2) | 0.9583 | 0.9165 | 1 |
| LysoPC(18:2) | Hex1Cer | SPH | 0.9583 | 0.9160 | 1 |
| LysoPC(18:2) | Hex2Cer | SPH | 0.9583 | 0.9145 | 1 |
| Taurodeoxycholic acid | Hex1Cer | ST | 0.9583 | 0.9180 | 0.9986 |
| Taurodeoxycholic acid | Hex1Cer | ST(d18:1/20:2) | 0.9583 | 0.9180 | 0.9986 |
| Glycocholic acid | Cholesterol sulfate | SPH | 0.9583 | 0.9160 | 1 |
| Glycocholic acid | Taurocholic acid | ST | 0.9583 | 0.9174 | 0.9992 |
| Glycocholic acid | Taurocholic acid | ST(d18:1/20:2) | 0.9583 | 0.9174 | 0.9992 |
| Cholesterol sulfate | CL | SPH | 0.9583 | 0.9167 | 0.9999 |
| FA | Hex1Cer | PC(18:1/24:2) | 0.9583 | 0.9100 | 1 |
| CerP | OAHFA | ST | 0.9579 | 0.9229 | 0.9929 |
| CerP | OAHFA | ST(d18:1/20:2) | 0.9579 | 0.9229 | 0.9929 |
| LysoPC(18:2) | AcHexSiE | PE(20:0p/20:3) | 0.9575 | 0.9091 | 1 |
| LysoPC(18:2) | DLCL | PC(18:1/24:2) | 0.9575 | 0.9134 | 1 |
| LysoPC(18:2) | GD1a | PC(18:1/24:2) | 0.9575 | 0.9056 | 1 |
| Glycocholic acid | OAHFA | PE(20:0p/20:3) | 0.9575 | 0.9177 | 0.9973 |
| AcHexSiE | DLCL | SPH | 0.9575 | 0.9135 | 1 |
| CerP | CL | SPH | 0.9575 | 0.9152 | 0.9998 |
| CerP | PG | PE(20:0p/20:3) | 0.9575 | 0.9231 | 0.9919 |
| CL | DLCL | ST | 0.9575 | 0.9262 | 0.9889 |
| CL | DLCL | ST(d18:1/20:2) | 0.9575 | 0.9262 | 0.9889 |
| FA | LysoPS | SPH | 0.9575 | 0.9165 | 0.9986 |
| CL | FA | PE(20:0p/20:3) | 0.9571 | 0.9224 | 0.9918 |
| Glycocholic acid | GD1a | PE(20:0p/20:3) | 0.9567 | 0.9150 | 0.9984 |
| Glycocholic acid | Hex2Cer | ST | 0.9567 | 0.9124 | 1 |
| Glycocholic acid | Hex2Cer | ST(d18:1/20:2) | 0.9567 | 0.9124 | 1 |
| Cholesterol sulfate | Taurocholic acid | PE(20:0p/20:3) | 0.9567 | 0.9144 | 0.9990 |
| Cholesterol sulfate | GD1a | ST | 0.9567 | 0.9102 | 1 |
| Cholesterol sulfate | GD1a | ST(d18:1/20:2) | 0.9567 | 0.9102 | 1 |
| Taurocholic acid | Hex2Cer | ST | 0.9567 | 0.9168 | 0.9967 |
| Taurocholic acid | Hex2Cer | ST(d18:1/20:2) | 0.9567 | 0.9168 | 0.9967 |
| Taurocholic acid | ST | ST(d18:1/20:2) | 0.9567 | 0.9160 | 0.9975 |
| AcHexSiE | CerP | SPH | 0.9567 | 0.9138 | 0.9997 |
| AcHexSiE | PG | SPH | 0.9567 | 0.9097 | 1 |
| CerP | Hex2Cer | PC(18:1/24:2) | 0.9567 | 0.9124 | 1 |
| GD1a | Hex1Cer | PC(18:1/24:2) | 0.9567 | 0.9120 | 1 |
| Cholesterol sulfate | FA | SPH | 0.9563 | 0.9128 | 0.9999 |
| CL | Hex1Cer | PE(20:0p/20:3) | 0.9563 | 0.9176 | 0.9950 |
| Cholesterol sulfate | FA | ST | 0.9559 | 0.9096 | 1 |
| Cholesterol sulfate | FA | ST(d18:1/20:2) | 0.9559 | 0.9096 | 1 |
| Cholesterol sulfate | ST | ST(d18:1/20:2) | 0.9559 | 0.9096 | 1 |
| Taurocholic acid | FA | ST | 0.9559 | 0.9131 | 0.9987 |
| Taurocholic acid | FA | ST(d18:1/20:2) | 0.9559 | 0.9131 | 0.9987 |
| CerPE | FA | SPH | 0.9559 | 0.9117 | 1 |
| CL | FA | SPH | 0.9559 | 0.9137 | 0.9981 |
| DLCL | Hex2Cer | SPH | 0.9559 | 0.9080 | 1 |
| CerP | OAHFA | PC(18:1/24:2) | 0.9555 | 0.9127 | 0.9984 |
| Taurodeoxycholic acid | Cholesterol sulfate | PE(20:0p/20:3) | 0.9552 | 0.9157 | 0.9947 |
| Glycocholic acid | CL | SPH | 0.9552 | 0.9102 | 1 |
| Glycocholic acid | FA | SPH | 0.9552 | 0.9106 | 0.9997 |
| AcHexSiE | CerPE | SPH | 0.9552 | 0.9073 | 1 |
| AcHexSiE | OAHFA | ST | 0.9552 | 0.9170 | 0.9933 |
| AcHexSiE | OAHFA | ST(d18:1/20:2) | 0.9552 | 0.9170 | 0.9933 |
| CerPE | OAHFA | ST | 0.9552 | 0.9167 | 0.9936 |
| CerPE | OAHFA | ST(d18:1/20:2) | 0.9552 | 0.9167 | 0.9936 |
| Cholesterol sulfate | GD1a | SPH | 0.9548 | 0.9101 | 0.9994 |
| Glycocholic acid | Taurocholic acid | PE(20:0p/20:3) | 0.9544 | 0.9115 | 0.9972 |
| Glycocholic acid | GD1a | SPH | 0.9544 | 0.9094 | 0.9993 |
| Glycocholic acid | GD1a | ST | 0.9544 | 0.9066 | 1 |
| Glycocholic acid | GD1a | ST(d18:1/20:2) | 0.9544 | 0.9066 | 1 |
| Cholesterol sulfate | CerPE | ST | 0.9544 | 0.9079 | 1 |
| Cholesterol sulfate | CerPE | ST(d18:1/20:2) | 0.9544 | 0.9079 | 1 |
| Cholesterol sulfate | Hex1Cer | ST | 0.9544 | 0.9073 | 1 |
| Cholesterol sulfate | Hex1Cer | ST(d18:1/20:2) | 0.9544 | 0.9073 | 1 |
| Cholesterol sulfate | OAHFA | PE(20:0p/20:3) | 0.9544 | 0.9126 | 0.9962 |
| Taurocholic acid | Hex1Cer | ST | 0.9544 | 0.9129 | 0.9959 |
| Taurocholic acid | Hex1Cer | ST(d18:1/20:2) | 0.9544 | 0.9129 | 0.9959 |
| CerP | FA | SPH | 0.9544 | 0.9101 | 0.9986 |
| CerP | LysoPS | SPH | 0.9544 | 0.9093 | 0.9994 |
| CL | PG | SPH | 0.9544 | 0.9071 | 1 |
| DLCL | GD1a | SPH | 0.9544 | 0.9083 | 1 |
| GD1a | OAHFA | ST | 0.9544 | 0.9154 | 0.9933 |
| GD1a | OAHFA | ST(d18:1/20:2) | 0.9544 | 0.9154 | 0.9933 |
| CL | ST | ST(d18:1/20:2) | 0.9540 | 0.9208 | 0.9871 |
| GD1a | PG | SPH | 0.9540 | 0.9037 | 1 |
| Glycocholic acid | CerPE | PE(20:0p/20:3) | 0.9536 | 0.9118 | 0.9954 |
| Glycocholic acid | ST | ST(d18:1/20:2) | 0.9536 | 0.9060 | 1 |
| Cholesterol sulfate | CerPE | PE(20:0p/20:3) | 0.9536 | 0.9113 | 0.9959 |
| AcHexSiE | GD1a | SPH | 0.9536 | 0.9076 | 0.9996 |
| AcHexSiE | OAHFA | SPH | 0.9536 | 0.9050 | 1 |
| CerP | GD1a | SPH | 0.9536 | 0.9086 | 0.9986 |
| CerPE | CL | SPH | 0.9536 | 0.9058 | 1 |
| CL | LysoPS | SPH | 0.9536 | 0.9072 | 0.9999 |
| Hex1Cer | OAHFA | PC(18:1/24:2) | 0.9536 | 0.9089 | 0.9983 |
| LysoPS | OAHFA | SPH | 0.9536 | 0.9052 | 1 |
| LysoPC(18:2) | Cholesterol sulfate | ST | 0.9528 | 0.9047 | 1 |
| LysoPC(18:2) | Cholesterol sulfate | ST(d18:1/20:2) | 0.9528 | 0.9047 | 1 |
| Glycocholic acid | Cholesterol sulfate | ST | 0.9528 | 0.9071 | 0.9985 |
| Glycocholic acid | Cholesterol sulfate | ST(d18:1/20:2) | 0.9528 | 0.9071 | 0.9985 |
| Glycocholic acid | DLCL | SPH | 0.9528 | 0.9025 | 1 |
| Glycocholic acid | FA | ST | 0.9528 | 0.9050 | 1 |
| Glycocholic acid | FA | ST(d18:1/20:2) | 0.9528 | 0.9050 | 1 |
| Taurocholic acid | CerPE | ST | 0.9528 | 0.9104 | 0.9952 |
| Taurocholic acid | CerPE | ST(d18:1/20:2) | 0.9528 | 0.9104 | 0.9952 |
| CerP | CerPE | PC(18:1/24:2) | 0.9528 | 0.9146 | 0.9910 |
| CerPE | GD1a | SPH | 0.9528 | 0.9035 | 1 |
| CL | GD1a | ST | 0.9528 | 0.9169 | 0.9887 |
| CL | GD1a | ST(d18:1/20:2) | 0.9528 | 0.9169 | 0.9887 |
| GD1a | OAHFA | PE(20:0p/20:3) | 0.9528 | 0.9072 | 0.9984 |
| Cholesterol sulfate | DLCL | SPH | 0.9524 | 0.9020 | 1 |
| Taurodeoxycholic acid | Glycocholic acid | PE(20:0p/20:3) | 0.9520 | 0.9107 | 0.9934 |
| Glycocholic acid | CerPE | ST | 0.9520 | 0.9045 | 0.9995 |
| Glycocholic acid | CerPE | ST(d18:1/20:2) | 0.9520 | 0.9045 | 0.9995 |
| Glycocholic acid | Hex1Cer | ST | 0.9520 | 0.9038 | 1 |
| Glycocholic acid | Hex1Cer | ST(d18:1/20:2) | 0.9520 | 0.9038 | 1 |
| DLCL | Hex1Cer | PC(18:1/24:2) | 0.9520 | 0.9088 | 0.9952 |
| FA | OAHFA | ST | 0.9520 | 0.9125 | 0.9915 |
| FA | OAHFA | ST(d18:1/20:2) | 0.9520 | 0.9125 | 0.9915 |
| AcHexSiE | FA | PE(20:0p/20:3) | 0.9516 | 0.9025 | 1 |
| FA | PG | SPH | 0.9516 | 0.9010 | 1 |
| LysoPC(18:2) | Glycocholic acid | ST | 0.9512 | 0.9010 | 1 |
| LysoPC(18:2) | Glycocholic acid | ST(d18:1/20:2) | 0.9512 | 0.9010 | 1 |
| Cholesterol sulfate | FA | PE(20:0p/20:3) | 0.9512 | 0.9055 | 0.9969 |
| Cholesterol sulfate | Hex1Cer | PE(20:0p/20:3) | 0.9512 | 0.9088 | 0.9936 |
| Taurocholic acid | DLCL | PE(20:0p/20:3) | 0.9512 | 0.9068 | 0.9956 |
| Taurocholic acid | FA | SPH | 0.9512 | 0.9020 | 1 |
| Taurocholic acid | GD1a | SPH | 0.9512 | 0.9013 | 1 |
| OAHFA | ST | ST(d18:1/20:2) | 0.9512 | 0.9117 | 0.9908 |
| Taurodeoxycholic acid | GD1a | SPH | 0.9504 | 0.8983 | 1 |
| Glycocholic acid | Cholesterol sulfate | PE(20:0p/20:3) | 0.9504 | 0.9046 | 0.9962 |
| Glycocholic acid | CerP | PE(20:0p/20:3) | 0.9504 | 0.9038 | 0.9970 |
| Cholesterol sulfate | CerP | PE(20:0p/20:3) | 0.9504 | 0.9042 | 0.9967 |
| LysoPC(18:2) | Hex1Cer | PC(18:1/24:2) | 0.9496 | 0.8985 | 1 |
| Taurodeoxycholic acid | FA | SPH | 0.9496 | 0.8975 | 1 |
| Glycocholic acid | Hex1Cer | PE(20:0p/20:3) | 0.9496 | 0.9063 | 0.9930 |
| Cholesterol sulfate | CerP | SPH | 0.9493 | 0.8973 | 1 |
| Cholesterol sulfate | CerPE | SPH | 0.9493 | 0.8939 | 1 |
| LysoPC(18:2) | Cholesterol sulfate | PE(20:0p/20:3) | 0.9489 | 0.9032 | 0.9945 |
| Taurodeoxycholic acid | Taurocholic acid | SPH | 0.9489 | 0.8945 | 1 |
| Glycocholic acid | CerP | SPH | 0.9489 | 0.8971 | 1 |
| Glycocholic acid | CerPE | SPH | 0.9489 | 0.8932 | 1 |
| AcHexSiE | GD1a | PE(20:0p/20:3) | 0.9489 | 0.9012 | 0.9965 |
| DLCL | GD1a | PE(20:0p/20:3) | 0.9489 | 0.9088 | 0.9890 |
| Glycocholic acid | FA | PE(20:0p/20:3) | 0.9481 | 0.9002 | 0.9960 |
| AcHexSiE | CerP | PC(18:1/24:2) | 0.9481 | 0.8996 | 0.9965 |
| CerP | DLCL | SPH | 0.9481 | 0.8942 | 1 |
| CerP | PG | SPH | 0.9481 | 0.8918 | 1 |
| CerPE | CL | PE(20:0p/20:3) | 0.9481 | 0.9080 | 0.9881 |
| Hex2Cer | OAHFA | ST | 0.9481 | 0.9035 | 0.9926 |
| Hex2Cer | OAHFA | ST(d18:1/20:2) | 0.9481 | 0.9035 | 0.9926 |
| LysoPC(18:2) | Glycocholic acid | PE(20:0p/20:3) | 0.9473 | 0.9008 | 0.9938 |
| LysoPC(18:2) | CL | PE(20:0p/20:3) | 0.9473 | 0.9029 | 0.9917 |
| Glycocholic acid | PG | SPH | 0.9473 | 0.8877 | 1 |
| Cholesterol sulfate | PG | SPH | 0.9473 | 0.8877 | 1 |
| Taurocholic acid | GD1a | PE(20:0p/20:3) | 0.9473 | 0.8924 | 1 |
| CerPE | Hex2Cer | SPH | 0.9473 | 0.8903 | 1 |
| DLCL | PG | SPH | 0.9473 | 0.8899 | 1 |
| GD1a | OAHFA | SPH | 0.9473 | 0.8945 | 1 |
| Taurodeoxycholic acid | DLCL | SPH | 0.9465 | 0.8883 | 1 |
| Taurodeoxycholic acid | PG | SPH | 0.9465 | 0.8835 | 1 |
| Taurocholic acid | DLCL | SPH | 0.9465 | 0.8888 | 1 |
| CerP | GD1a | PC(18:1/24:2) | 0.9465 | 0.8970 | 0.9960 |
| Hex1Cer | PG | SPH | 0.9465 | 0.8898 | 1 |
| LysoPC(18:2) | FA | PC(18:1/24:2) | 0.9461 | 0.8876 | 1 |
| Taurodeoxycholic acid | CL | SPH | 0.9457 | 0.8877 | 1 |
| Taurocholic acid | CL | SPH | 0.9457 | 0.8897 | 1 |
| Taurocholic acid | PG | SPH | 0.9457 | 0.8832 | 1 |
| CerP | DLCL | PC(18:1/24:2) | 0.9457 | 0.9026 | 0.9888 |
| CerPE | PG | SPH | 0.9457 | 0.8861 | 1 |
| DLCL | FA | ST | 0.9457 | 0.9020 | 0.9895 |
| DLCL | FA | ST(d18:1/20:2) | 0.9457 | 0.9020 | 0.9895 |
| FA | OAHFA | SPH | 0.9457 | 0.8937 | 0.9978 |
| Hex1Cer | LysoPS | SPH | 0.9457 | 0.8870 | 1 |
| Hex2Cer | PG | SPH | 0.9457 | 0.8864 | 1 |
| CerP | FA | PC(18:1/24:2) | 0.9453 | 0.8942 | 0.9964 |
| LysoPC(18:2) | LysoPS | PG | 0.9449 | 0.8973 | 0.9926 |
| Glycocholic acid | Hex1Cer | SPH | 0.9449 | 0.8889 | 1 |
| CerP | CerPE | SPH | 0.9449 | 0.8872 | 1 |
| DLCL | Hex1Cer | SPH | 0.9449 | 0.8861 | 1 |
| LysoPC(18:2) | AcHexSiE | ST | 0.9441 | 0.8876 | 1 |
| LysoPC(18:2) | AcHexSiE | ST(d18:1/20:2) | 0.9441 | 0.8876 | 1 |
| LysoPC(18:2) | CerP | PC(18:1/24:2) | 0.9441 | 0.8868 | 1 |
| Taurodeoxycholic acid | Cholesterol sulfate | SPH | 0.9441 | 0.8860 | 1 |
| Taurodeoxycholic acid | CerPE | SPH | 0.9441 | 0.8809 | 1 |
| AcHexSiE | FA | SPH | 0.9441 | 0.8874 | 1 |
| CL | OAHFA | SPH | 0.9441 | 0.8898 | 0.9985 |
| Hex1Cer | OAHFA | ST | 0.9441 | 0.8979 | 0.9904 |
| Hex1Cer | OAHFA | ST(d18:1/20:2) | 0.9441 | 0.8979 | 0.9904 |
| DLCL | OAHFA | PE(20:0p/20:3) | 0.9437 | 0.8992 | 0.9883 |
| Taurodeoxycholic acid | Glycocholic acid | SPH | 0.9434 | 0.8848 | 1 |
| AcHexSiE | CerPE | PE(20:0p/20:3) | 0.9434 | 0.8867 | 1 |
| CerP | CL | PE(20:0p/20:3) | 0.9434 | 0.9012 | 0.9855 |
| CerPE | DLCL | SPH | 0.9434 | 0.8829 | 1 |
| Hex2Cer | LysoPS | SPH | 0.9434 | 0.8779 | 1 |
| Taurocholic acid | CerPE | SPH | 0.9426 | 0.8795 | 1 |
| AcHexSiE | Hex1Cer | SPH | 0.9426 | 0.8843 | 1 |
| Glycocholic acid | OAHFA | SPH | 0.9418 | 0.8822 | 1 |
| Taurocholic acid | CerP | SPH | 0.9418 | 0.8813 | 1 |
| Taurocholic acid | Hex1Cer | SPH | 0.9418 | 0.8802 | 1 |
| DLCL | LysoPS | PG | 0.9418 | 0.8938 | 0.9898 |
| FA | Hex2Cer | ST | 0.9418 | 0.8928 | 0.9907 |
| FA | Hex2Cer | ST(d18:1/20:2) | 0.9418 | 0.8928 | 0.9907 |
| Cholesterol sulfate | OAHFA | SPH | 0.9414 | 0.8817 | 1 |
| Taurodeoxycholic acid | CerP | SPH | 0.9410 | 0.8802 | 1 |
| AcHexSiE | GD1a | PC(18:1/24:2) | 0.9410 | 0.8918 | 0.9902 |
| CerP | FA | ST | 0.9410 | 0.8937 | 0.9883 |
| CerP | FA | ST(d18:1/20:2) | 0.9410 | 0.8937 | 0.9883 |
| DLCL | FA | LysoPS | 0.9410 | 0.8869 | 0.9951 |
| FA | GD1a | SPH | 0.9410 | 0.8809 | 1 |
| Taurodeoxycholic acid | OAHFA | SPH | 0.9402 | 0.8746 | 1 |
| Cholesterol sulfate | Hex1Cer | SPH | 0.9402 | 0.8795 | 1 |
| AcHexSiE | Hex2Cer | SPH | 0.9402 | 0.8798 | 1 |
| CerP | DLCL | ST | 0.9398 | 0.8996 | 0.9800 |
| CerP | DLCL | ST(d18:1/20:2) | 0.9398 | 0.8996 | 0.9800 |
| LysoPC(18:2) | Hex2Cer | ST | 0.9394 | 0.8845 | 0.9944 |
| LysoPC(18:2) | Hex2Cer | ST(d18:1/20:2) | 0.9394 | 0.8845 | 0.9944 |
| Taurodeoxycholic acid | Hex1Cer | SPH | 0.9394 | 0.8774 | 1 |
| CerP | Hex1Cer | PC(18:1/24:2) | 0.9394 | 0.8880 | 0.9908 |
| OAHFA | PG | SPH | 0.9394 | 0.8755 | 1 |
| Taurodeoxycholic acid | DLCL | PE(20:0p/20:3) | 0.9386 | 0.8851 | 0.9921 |
| Glycocholic acid | LysoPS | SPH | 0.9386 | 0.8690 | 1 |
| Cholesterol sulfate | LysoPS | SPH | 0.9386 | 0.8690 | 1 |
| AcHexSiE | LysoPS | PG | 0.9386 | 0.8861 | 0.9912 |
| CerPE | DLCL | ST | 0.9386 | 0.8904 | 0.9869 |
| CerPE | DLCL | ST(d18:1/20:2) | 0.9386 | 0.8904 | 0.9869 |
| CerPE | Hex1Cer | SPH | 0.9386 | 0.8747 | 1 |
| FA | Hex1Cer | SPH | 0.9386 | 0.8782 | 0.9991 |
| Glycocholic acid | Taurocholic acid | SPH | 0.9378 | 0.8714 | 1 |
| Taurocholic acid | OAHFA | SPH | 0.9378 | 0.8724 | 1 |
| FA | Hex2Cer | SPH | 0.9378 | 0.8741 | 1 |
| LysoPC(18:2) | DLCL | PE(20:0p/20:3) | 0.9371 | 0.8857 | 0.9885 |
| Taurodeoxycholic acid | Hex2Cer | SPH | 0.9371 | 0.8711 | 1 |
| Cholesterol sulfate | Taurocholic acid | SPH | 0.9371 | 0.8691 | 1 |
| Taurocholic acid | Hex2Cer | SPH | 0.9371 | 0.8698 | 1 |
| AcHexSiE | DLCL | ST | 0.9371 | 0.8941 | 0.9801 |
| AcHexSiE | DLCL | ST(d18:1/20:2) | 0.9371 | 0.8941 | 0.9801 |
| GD1a | Hex2Cer | PE(20:0p/20:3) | 0.9367 | 0.8798 | 0.9935 |
| LysoPC(18:2) | GD1a | PE(20:0p/20:3) | 0.9363 | 0.8791 | 0.9935 |
| DLCL | OAHFA | SPH | 0.9363 | 0.8722 | 1 |
| LysoPC(18:2) | FA | ST | 0.9355 | 0.8751 | 0.9959 |
| LysoPC(18:2) | FA | ST(d18:1/20:2) | 0.9355 | 0.8751 | 0.9959 |
| Taurodeoxycholic acid | GD1a | PE(20:0p/20:3) | 0.9355 | 0.8738 | 0.9972 |
| AcHexSiE | CerP | PE(20:0p/20:3) | 0.9355 | 0.8741 | 0.9968 |
| CerPE | OAHFA | SPH | 0.9355 | 0.8690 | 1 |
| GD1a | Hex1Cer | SPH | 0.9355 | 0.8720 | 0.9990 |
| DLCL | FA | PE(20:0p/20:3) | 0.9351 | 0.8875 | 0.9827 |
| FA | OAHFA | PE(20:0p/20:3) | 0.9351 | 0.8778 | 0.9923 |
| LysoPC(18:2) | CerPE | ST | 0.9347 | 0.8751 | 0.9943 |
| LysoPC(18:2) | CerPE | ST(d18:1/20:2) | 0.9347 | 0.8751 | 0.9943 |
| LysoPC(18:2) | ST | ST(d18:1/20:2) | 0.9347 | 0.8751 | 0.9943 |
| AcHexSiE | LysoPS | OAHFA | 0.9347 | 0.8782 | 0.9912 |
| CL | LysoPS | PG | 0.9347 | 0.8828 | 0.9866 |
| Glycocholic acid | Hex2Cer | SPH | 0.9339 | 0.8672 | 1 |
| AcHexSiE | CerP | ST | 0.9339 | 0.8892 | 0.9786 |
| AcHexSiE | CerP | ST(d18:1/20:2) | 0.9339 | 0.8892 | 0.9786 |
| AcHexSiE | Hex1Cer | PE(20:0p/20:3) | 0.9335 | 0.8700 | 0.9970 |
| AcHexSiE | DLCL | LysoPS | 0.9335 | 0.8785 | 0.9886 |
| LysoPC(18:2) | GD1a | ST | 0.9331 | 0.8729 | 0.9933 |
| LysoPC(18:2) | GD1a | ST(d18:1/20:2) | 0.9331 | 0.8729 | 0.9933 |
| LysoPC(18:2) | Hex1Cer | ST | 0.9331 | 0.8698 | 0.9965 |
| LysoPC(18:2) | Hex1Cer | ST(d18:1/20:2) | 0.9331 | 0.8698 | 0.9965 |
| AcHexSiE | CL | LysoPS | 0.9331 | 0.8796 | 0.9867 |
| CerP | CerPE | ST | 0.9331 | 0.8813 | 0.9849 |
| CerP | CerPE | ST(d18:1/20:2) | 0.9331 | 0.8813 | 0.9849 |
| DLCL | GD1a | ST | 0.9331 | 0.8873 | 0.9789 |
| DLCL | GD1a | ST(d18:1/20:2) | 0.9331 | 0.8873 | 0.9789 |
| Cholesterol sulfate | Hex2Cer | SPH | 0.9323 | 0.8643 | 1 |
| CerP | Hex2Cer | ST | 0.9323 | 0.8780 | 0.9867 |
| CerP | Hex2Cer | ST(d18:1/20:2) | 0.9323 | 0.8780 | 0.9867 |
| CerP | OAHFA | SPH | 0.9323 | 0.8645 | 1 |
| GD1a | Hex2Cer | SPH | 0.9323 | 0.8653 | 0.9994 |
| LysoPC(18:2) | DLCL | LysoPS | 0.9315 | 0.8688 | 0.9943 |
| DLCL | Hex2Cer | LysoPS | 0.9315 | 0.8769 | 0.9862 |
| LysoPS | OAHFA | PG | 0.9315 | 0.8767 | 0.9864 |
| LysoPC(18:2) | OAHFA | PE(20:0p/20:3) | 0.9308 | 0.8705 | 0.9910 |
| DLCL | Hex1Cer | LysoPS | 0.9304 | 0.8752 | 0.9856 |
| Taurodeoxycholic acid | AcHexSiE | LysoPS | 0.9300 | 0.8750 | 0.9850 |
| CerP | GD1a | ST | 0.9300 | 0.8826 | 0.9774 |
| CerP | GD1a | ST(d18:1/20:2) | 0.9300 | 0.8826 | 0.9774 |
| DLCL | Hex2Cer | PE(20:0p/20:3) | 0.9296 | 0.8743 | 0.9849 |
| Hex2Cer | OAHFA | PE(20:0p/20:3) | 0.9296 | 0.8732 | 0.9860 |
| Taurocholic acid | OAHFA | PE(20:0p/20:3) | 0.9292 | 0.8703 | 0.9881 |
| CerPE | Hex2Cer | ST | 0.9292 | 0.8730 | 0.9854 |
| CerPE | Hex2Cer | ST(d18:1/20:2) | 0.9292 | 0.8730 | 0.9854 |
| DLCL | Hex1Cer | ST | 0.9292 | 0.8740 | 0.9844 |
| DLCL | Hex1Cer | ST(d18:1/20:2) | 0.9292 | 0.8740 | 0.9844 |
| DLCL | ST | ST(d18:1/20:2) | 0.9292 | 0.8837 | 0.9746 |
| CerP | Hex1Cer | SPH | 0.9284 | 0.8547 | 1 |
| CL | DLCL | SPH | 0.9284 | 0.8496 | 1 |
| LysoPC(18:2) | Hex2Cer | PE(20:0p/20:3) | 0.9276 | 0.8704 | 0.9848 |
| AcHexSiE | Hex2Cer | ST | 0.9276 | 0.8664 | 0.9888 |
| AcHexSiE | Hex2Cer | ST(d18:1/20:2) | 0.9276 | 0.8664 | 0.9888 |
| CerP | OAHFA | PE(20:0p/20:3) | 0.9276 | 0.8691 | 0.9861 |
| CerPE | LysoPS | PG | 0.9276 | 0.8713 | 0.9839 |
| Taurodeoxycholic acid | LysoPS | PG | 0.9268 | 0.8663 | 0.9874 |
| Taurocholic acid | Hex2Cer | PE(20:0p/20:3) | 0.9268 | 0.8670 | 0.9866 |
| CerP | Hex1Cer | ST | 0.9268 | 0.8699 | 0.9837 |
| CerP | Hex1Cer | ST(d18:1/20:2) | 0.9268 | 0.8699 | 0.9837 |
| DLCL | Hex2Cer | ST | 0.9268 | 0.8686 | 0.9851 |
| DLCL | Hex2Cer | ST(d18:1/20:2) | 0.9268 | 0.8686 | 0.9851 |
| Taurocholic acid | DLCL | LysoPS | 0.9260 | 0.8633 | 0.9887 |
| CL | DLCL | LysoPS | 0.9260 | 0.8653 | 0.9868 |
| FA | Hex1Cer | ST | 0.9260 | 0.8658 | 0.9863 |
| FA | Hex1Cer | ST(d18:1/20:2) | 0.9260 | 0.8658 | 0.9863 |
| FA | Hex2Cer | PE(20:0p/20:3) | 0.9260 | 0.8689 | 0.9832 |
| CerP | ST | ST(d18:1/20:2) | 0.9256 | 0.8785 | 0.9728 |
| Taurodeoxycholic acid | Taurocholic acid | PE(20:0p/20:3) | 0.9253 | 0.8602 | 0.9903 |
| Taurocholic acid | LysoPS | PG | 0.9253 | 0.8643 | 0.9862 |
| LysoPC(18:2) | AcHexSiE | OAHFA | 0.9245 | 0.8680 | 0.9809 |
| CL | Hex1Cer | SPH | 0.9245 | 0.8481 | 1 |
| Taurodeoxycholic acid | DLCL | LysoPS | 0.9237 | 0.8581 | 0.9892 |
| Taurodeoxycholic acid | Hex2Cer | PE(20:0p/20:3) | 0.9237 | 0.8627 | 0.9847 |
| Taurodeoxycholic acid | OAHFA | PE(20:0p/20:3) | 0.9237 | 0.8624 | 0.9850 |
| Cholesterol sulfate | LysoPS | PG | 0.9237 | 0.8646 | 0.9828 |
| Hex1Cer | LysoPS | PG | 0.9229 | 0.8622 | 0.9836 |
| AcHexSiE | FA | ST | 0.9225 | 0.8609 | 0.9841 |
| AcHexSiE | FA | ST(d18:1/20:2) | 0.9225 | 0.8609 | 0.9841 |
| Glycocholic acid | AcHexSiE | LysoPS | 0.9221 | 0.8632 | 0.9810 |
| Taurocholic acid | CerPE | PE(20:0p/20:3) | 0.9221 | 0.8556 | 0.9886 |
| CerPE | GD1a | PE(20:0p/20:3) | 0.9221 | 0.8560 | 0.9882 |
| CerPE | OAHFA | PE(20:0p/20:3) | 0.9221 | 0.8612 | 0.9830 |
| FA | LysoPS | PG | 0.9221 | 0.8619 | 0.9823 |
| Hex1Cer | OAHFA | SPH | 0.9221 | 0.8436 | 1 |
| Hex1Cer | OAHFA | PE(20:0p/20:3) | 0.9221 | 0.8607 | 0.9835 |
| Hex2Cer | LysoPS | PG | 0.9221 | 0.8614 | 0.9828 |
| Glycocholic acid | LysoPS | PG | 0.9213 | 0.8615 | 0.9812 |
| Taurocholic acid | FA | PE(20:0p/20:3) | 0.9213 | 0.8551 | 0.9875 |
| GD1a | Hex1Cer | PE(20:0p/20:3) | 0.9209 | 0.8520 | 0.9899 |
| Taurocholic acid | AcHexSiE | LysoPS | 0.9205 | 0.8610 | 0.9801 |
| Taurocholic acid | CerP | PE(20:0p/20:3) | 0.9205 | 0.8564 | 0.9846 |
| CL | GD1a | LysoPS | 0.9201 | 0.8606 | 0.9797 |
| CerP | LysoPS | PG | 0.9197 | 0.8574 | 0.9821 |
| CerPE | Hex1Cer | ST | 0.9197 | 0.8561 | 0.9834 |
| CerPE | Hex1Cer | ST(d18:1/20:2) | 0.9197 | 0.8561 | 0.9834 |
| DLCL | GD1a | LysoPS | 0.9197 | 0.8582 | 0.9813 |
| DLCL | Hex1Cer | PE(20:0p/20:3) | 0.9197 | 0.8595 | 0.9800 |
| GD1a | LysoPS | PG | 0.9197 | 0.8580 | 0.9815 |
| Cholesterol sulfate | AcHexSiE | LysoPS | 0.9190 | 0.8579 | 0.9800 |
| CerPE | DLCL | LysoPS | 0.9190 | 0.8520 | 0.9859 |
| CerPE | DLCL | PE(20:0p/20:3) | 0.9190 | 0.8633 | 0.9746 |
| Hex1Cer | Hex2Cer | ST | 0.9190 | 0.8523 | 0.9856 |
| Hex1Cer | Hex2Cer | ST(d18:1/20:2) | 0.9190 | 0.8523 | 0.9856 |
| CerP | DLCL | PE(20:0p/20:3) | 0.9186 | 0.8651 | 0.9720 |
| FA | GD1a | ST | 0.9178 | 0.8553 | 0.9803 |
| FA | GD1a | ST(d18:1/20:2) | 0.9178 | 0.8553 | 0.9803 |
| LysoPC(18:2) | AcHexSiE | LysoPS | 0.9174 | 0.8513 | 0.9835 |
| CerPE | CL | LysoPS | 0.9166 | 0.8505 | 0.9827 |
| CerP | Hex2Cer | SPH | 0.9158 | 0.8303 | 1 |
| GD1a | Hex2Cer | ST | 0.9158 | 0.8497 | 0.9819 |
| GD1a | Hex2Cer | ST(d18:1/20:2) | 0.9158 | 0.8497 | 0.9819 |
| AcHexSiE | Hex1Cer | ST | 0.9154 | 0.8484 | 0.9824 |
| AcHexSiE | Hex1Cer | ST(d18:1/20:2) | 0.9154 | 0.8484 | 0.9824 |
| CerP | GD1a | PE(20:0p/20:3) | 0.9150 | 0.8454 | 0.9846 |
| CerPE | Hex2Cer | PE(20:0p/20:3) | 0.9150 | 0.8525 | 0.9776 |
| Hex1Cer | Hex2Cer | SPH | 0.9150 | 0.8298 | 1 |
| Hex1Cer | Hex2Cer | PE(20:0p/20:3) | 0.9150 | 0.8474 | 0.9827 |
| Hex2Cer | OAHFA | SPH | 0.9150 | 0.8299 | 1 |
| Hex2Cer | ST | ST(d18:1/20:2) | 0.9150 | 0.8491 | 0.9810 |
| CerPE | CL | PC(18:1/24:2) | 0.9146 | 0.8563 | 0.9729 |
| CL | DLCL | PC(18:1/24:2) | 0.9146 | 0.8563 | 0.9729 |
| LysoPC(18:2) | Taurocholic acid | PE(20:0p/20:3) | 0.9142 | 0.8463 | 0.9822 |
| LysoPC(18:2) | CerPE | LysoPS | 0.9142 | 0.8478 | 0.9807 |
| AcHexSiE | CerPE | LysoPS | 0.9142 | 0.8499 | 0.9786 |
| CL | Hex2Cer | SPH | 0.9142 | 0.8283 | 1 |
| CerP | Hex2Cer | PE(20:0p/20:3) | 0.9138 | 0.8485 | 0.9792 |
| LysoPC(18:2) | Hex1Cer | PE(20:0p/20:3) | 0.9135 | 0.8463 | 0.9807 |
| Taurodeoxycholic acid | Hex1Cer | PE(20:0p/20:3) | 0.9127 | 0.8487 | 0.9766 |
| LysoPC(18:2) | CL | LysoPS | 0.9119 | 0.8419 | 0.9819 |
| Taurocholic acid | Hex1Cer | PE(20:0p/20:3) | 0.9119 | 0.8473 | 0.9764 |
| CL | LysoPS | OAHFA | 0.9119 | 0.8435 | 0.9803 |
| Taurodeoxycholic acid | CerPE | LysoPS | 0.9111 | 0.8453 | 0.9769 |
| Taurodeoxycholic acid | CL | LysoPS | 0.9111 | 0.8398 | 0.9824 |
| Taurocholic acid | CL | LysoPS | 0.9111 | 0.8414 | 0.9808 |
| AcHexSiE | FA | OAHFA | 0.9111 | 0.8530 | 0.9692 |
| AcHexSiE | GD1a | ST | 0.9111 | 0.8506 | 0.9716 |
| AcHexSiE | GD1a | ST(d18:1/20:2) | 0.9111 | 0.8506 | 0.9716 |
| FA | GD1a | PE(20:0p/20:3) | 0.9111 | 0.8394 | 0.9827 |
| GD1a | Hex1Cer | ST | 0.9107 | 0.8425 | 0.9789 |
| GD1a | Hex1Cer | ST(d18:1/20:2) | 0.9107 | 0.8425 | 0.9789 |
| CerP | CL | LysoPS | 0.9103 | 0.8428 | 0.9778 |
| Hex1Cer | ST | ST(d18:1/20:2) | 0.9099 | 0.8417 | 0.9781 |
| LysoPC(18:2) | LysoPS | OAHFA | 0.9095 | 0.8390 | 0.9800 |
| Glycocholic acid | CerPE | LysoPS | 0.9095 | 0.8432 | 0.9758 |
| FA | Hex1Cer | PE(20:0p/20:3) | 0.9095 | 0.8395 | 0.9796 |
| CL | FA | LysoPS | 0.9091 | 0.8404 | 0.9778 |
| DLCL | LysoPS | OAHFA | 0.9091 | 0.8346 | 0.9837 |
| Taurodeoxycholic acid | CerP | PE(20:0p/20:3) | 0.9087 | 0.8351 | 0.9824 |
| Taurodeoxycholic acid | CerPE | PE(20:0p/20:3) | 0.9087 | 0.8368 | 0.9807 |
| Cholesterol sulfate | CerPE | LysoPS | 0.9079 | 0.8410 | 0.9749 |
| Taurocholic acid | CerPE | LysoPS | 0.9079 | 0.8405 | 0.9754 |
| AcHexSiE | CerPE | ST | 0.9079 | 0.8373 | 0.9786 |
| AcHexSiE | CerPE | ST(d18:1/20:2) | 0.9079 | 0.8373 | 0.9786 |
| FA | ST | ST(d18:1/20:2) | 0.9079 | 0.8413 | 0.9746 |
| LysoPC(18:2) | Hex2Cer | LysoPS | 0.9072 | 0.8316 | 0.9827 |
| Glycocholic acid | AcHexSiE | OAHFA | 0.9072 | 0.8418 | 0.9725 |
| CerPE | FA | LysoPS | 0.9072 | 0.8399 | 0.9745 |
| LysoPC(18:2) | Taurodeoxycholic acid | PE(20:0p/20:3) | 0.9064 | 0.8338 | 0.9789 |
| AcHexSiE | Hex2Cer | LysoPS | 0.9064 | 0.8336 | 0.9792 |
| FA | Hex2Cer | LysoPS | 0.9064 | 0.8263 | 0.9865 |
| CerP | DLCL | LysoPS | 0.9060 | 0.8321 | 0.9799 |
| CerPE | FA | PE(20:0p/20:3) | 0.9060 | 0.8404 | 0.9715 |
| Hex1Cer | LysoPS | OAHFA | 0.9060 | 0.8391 | 0.9729 |
| LysoPC(18:2) | CerPE | PE(20:0p/20:3) | 0.9052 | 0.8333 | 0.9771 |
| Cholesterol sulfate | AcHexSiE | OAHFA | 0.9052 | 0.8391 | 0.9713 |
| GD1a | LysoPS | OAHFA | 0.9052 | 0.8356 | 0.9747 |
| Taurodeoxycholic acid | FA | PE(20:0p/20:3) | 0.9048 | 0.8275 | 0.9821 |
| CerPE | LysoPS | OAHFA | 0.9048 | 0.8318 | 0.9778 |
| Taurodeoxycholic acid | LysoPS | OAHFA | 0.9040 | 0.8287 | 0.9794 |
| LysoPC(18:2) | FA | PE(20:0p/20:3) | 0.9036 | 0.8301 | 0.9772 |
| AcHexSiE | CerP | LysoPS | 0.9028 | 0.8343 | 0.9714 |
| AcHexSiE | ST | ST(d18:1/20:2) | 0.9024 | 0.8410 | 0.9638 |
| Taurodeoxycholic acid | Hex2Cer | LysoPS | 0.9017 | 0.8225 | 0.9808 |
| Taurocholic acid | LysoPS | OAHFA | 0.9017 | 0.8274 | 0.9759 |
| AcHexSiE | GD1a | LysoPS | 0.9009 | 0.8330 | 0.9687 |
| CerPE | GD1a | ST | 0.9005 | 0.8274 | 0.9735 |
| CerPE | GD1a | ST(d18:1/20:2) | 0.9005 | 0.8274 | 0.9735 |
| DLCL | FA | OAHFA | 0.9001 | 0.8368 | 0.9633 |
| AcHexSiE | FA | LysoPS | 0.8993 | 0.8269 | 0.9716 |
| GD1a | ST | ST(d18:1/20:2) | 0.8985 | 0.8348 | 0.9623 |
| AcHexSiE | CL | OAHFA | 0.8981 | 0.8297 | 0.9665 |
| LysoPC(18:2) | Hex1Cer | LysoPS | 0.8977 | 0.8197 | 0.9757 |
| Taurodeoxycholic acid | AcHexSiE | OAHFA | 0.8977 | 0.8270 | 0.9684 |
| Taurocholic acid | Hex2Cer | LysoPS | 0.8977 | 0.8169 | 0.9785 |
| CerP | Hex1Cer | PE(20:0p/20:3) | 0.8969 | 0.8195 | 0.9744 |
| FA | LysoPS | OAHFA | 0.8969 | 0.8239 | 0.9700 |
| CerPE | Hex1Cer | PE(20:0p/20:3) | 0.8954 | 0.8200 | 0.9707 |
| AcHexSiE | Hex1Cer | LysoPS | 0.8950 | 0.8172 | 0.9728 |
| Taurocholic acid | GD1a | LysoPS | 0.8938 | 0.8202 | 0.9674 |
| LysoPC(18:2) | Taurodeoxycholic acid | LysoPS | 0.8930 | 0.8120 | 0.9740 |
| Taurodeoxycholic acid | GD1a | LysoPS | 0.8930 | 0.8190 | 0.9670 |
| Taurocholic acid | AcHexSiE | OAHFA | 0.8926 | 0.8199 | 0.9653 |
| Taurodeoxycholic acid | AcHexSiE | CL | 0.8922 | 0.8205 | 0.9639 |
| CerPE | GD1a | LysoPS | 0.8922 | 0.8179 | 0.9666 |
| CerPE | Hex1Cer | LysoPS | 0.8922 | 0.8192 | 0.9652 |
| LysoPC(18:2) | AcHexSiE | DLCL | 0.8918 | 0.8194 | 0.9642 |
| LysoPC(18:2) | Taurocholic acid | LysoPS | 0.8914 | 0.8059 | 0.9769 |
| CerPE | ST | ST(d18:1/20:2) | 0.8914 | 0.8151 | 0.9677 |
| Glycocholic acid | Cholesterol sulfate | LysoPS | 0.8906 | 0.8176 | 0.9637 |
| CerPE | FA | ST | 0.8906 | 0.8100 | 0.9712 |
| CerPE | FA | ST(d18:1/20:2) | 0.8906 | 0.8100 | 0.9712 |
| LysoPC(18:2) | DLCL | OAHFA | 0.8899 | 0.8132 | 0.9665 |
| Taurodeoxycholic acid | AcHexSiE | DLCL | 0.8887 | 0.8169 | 0.9605 |
| Taurodeoxycholic acid | Taurocholic acid | LysoPS | 0.8883 | 0.8122 | 0.9643 |
| Cholesterol sulfate | DLCL | LysoPS | 0.8883 | 0.7941 | 0.9825 |
| AcHexSiE | OAHFA | PG | 0.8883 | 0.8145 | 0.9620 |
| Taurodeoxycholic acid | Hex1Cer | LysoPS | 0.8867 | 0.8017 | 0.9717 |
| Taurocholic acid | CerP | LysoPS | 0.8867 | 0.8079 | 0.9655 |
| CerP | CerPE | LysoPS | 0.8867 | 0.8068 | 0.9666 |
| Glycocholic acid | DLCL | LysoPS | 0.8859 | 0.7903 | 0.9816 |
| FA | Hex1Cer | LysoPS | 0.8859 | 0.8000 | 0.9719 |
| Taurodeoxycholic acid | CerP | LysoPS | 0.8851 | 0.8058 | 0.9645 |
| CerP | FA | PE(20:0p/20:3) | 0.8851 | 0.8041 | 0.9662 |
| CerP | LysoPS | OAHFA | 0.8851 | 0.8026 | 0.9676 |
| Taurocholic acid | Hex1Cer | LysoPS | 0.8843 | 0.7973 | 0.9714 |
| LysoPC(18:2) | FA | LysoPS | 0.8836 | 0.8000 | 0.9671 |
| GD1a | Hex2Cer | LysoPS | 0.8836 | 0.7998 | 0.9673 |
| CerP | CerPE | PE(20:0p/20:3) | 0.8832 | 0.8055 | 0.9608 |
| LysoPC(18:2) | CerP | PE(20:0p/20:3) | 0.8820 | 0.8011 | 0.9628 |
| LysoPC(18:2) | GD1a | LysoPS | 0.8820 | 0.7985 | 0.9655 |
| Taurodeoxycholic acid | AcHexSiE | PG | 0.8820 | 0.8023 | 0.9617 |
| CL | Hex2Cer | LysoPS | 0.8820 | 0.7884 | 0.9755 |
| LysoPC(18:2) | CL | OAHFA | 0.8812 | 0.7991 | 0.9633 |
| CerP | GD1a | LysoPS | 0.8812 | 0.8034 | 0.9590 |
| Hex1Cer | Hex2Cer | LysoPS | 0.8812 | 0.7959 | 0.9665 |
| LysoPC(18:2) | AcHexSiE | Hex2Cer | 0.8804 | 0.8047 | 0.9561 |
| LysoPC(18:2) | CerP | LysoPS | 0.8804 | 0.7924 | 0.9684 |
| FA | Hex2Cer | OAHFA | 0.8804 | 0.8014 | 0.9594 |
| LysoPC(18:2) | Glycocholic acid | LysoPS | 0.8796 | 0.7909 | 0.9683 |
| LysoPC(18:2) | Cholesterol sulfate | LysoPS | 0.8796 | 0.7909 | 0.9683 |
| AcHexSiE | DLCL | OAHFA | 0.8792 | 0.8082 | 0.9503 |
| Glycocholic acid | FA | LysoPS | 0.8788 | 0.7903 | 0.9674 |
| CL | FA | OAHFA | 0.8788 | 0.8030 | 0.9547 |
| Taurocholic acid | FA | LysoPS | 0.8780 | 0.7877 | 0.9684 |
| CL | GD1a | OAHFA | 0.8777 | 0.8013 | 0.9540 |
| Taurodeoxycholic acid | Glycocholic acid | LysoPS | 0.8773 | 0.7905 | 0.9641 |
| AcHexSiE | GD1a | OAHFA | 0.8773 | 0.8092 | 0.9453 |
| CL | Hex1Cer | LysoPS | 0.8773 | 0.7818 | 0.9727 |
| FA | GD1a | OAHFA | 0.8773 | 0.8041 | 0.9504 |
| LysoPC(18:2) | OAHFA | PG | 0.8765 | 0.7928 | 0.9601 |
| FA | GD1a | LysoPS | 0.8765 | 0.7919 | 0.9610 |
| Glycocholic acid | AcHexSiE | DLCL | 0.8741 | 0.7971 | 0.9511 |
| Cholesterol sulfate | AcHexSiE | DLCL | 0.8737 | 0.7967 | 0.9507 |
| Taurodeoxycholic acid | FA | LysoPS | 0.8733 | 0.7828 | 0.9639 |
| Cholesterol sulfate | CL | LysoPS | 0.8733 | 0.7753 | 0.9714 |
| Hex2Cer | LysoPS | OAHFA | 0.8733 | 0.7789 | 0.9678 |
| Taurocholic acid | AcHexSiE | CL | 0.8729 | 0.7934 | 0.9525 |
| Glycocholic acid | AcHexSiE | CL | 0.8725 | 0.7941 | 0.9510 |
| CerPE | Hex2Cer | LysoPS | 0.8725 | 0.7807 | 0.9644 |
| LysoPC(18:2) | Taurodeoxycholic acid | AcHexSiE | 0.8718 | 0.7927 | 0.9508 |
| LysoPC(18:2) | FA | OAHFA | 0.8718 | 0.7903 | 0.9532 |
| Glycocholic acid | CL | LysoPS | 0.8718 | 0.7727 | 0.9708 |
| Cholesterol sulfate | LysoPS | OAHFA | 0.8718 | 0.7754 | 0.9681 |
| GD1a | Hex1Cer | LysoPS | 0.8714 | 0.7841 | 0.9586 |
| Taurodeoxycholic acid | Cholesterol sulfate | LysoPS | 0.8710 | 0.7804 | 0.9615 |
| Cholesterol sulfate | AcHexSiE | CL | 0.8710 | 0.7922 | 0.9498 |
| Taurodeoxycholic acid | Taurocholic acid | AcHexSiE | 0.8702 | 0.7899 | 0.9505 |
| Taurocholic acid | AcHexSiE | DLCL | 0.8698 | 0.7905 | 0.9491 |
| LysoPC(18:2) | AcHexSiE | CL | 0.8690 | 0.7889 | 0.9491 |
| Glycocholic acid | LysoPS | OAHFA | 0.8686 | 0.7706 | 0.9666 |
| LysoPC(18:2) | GD1a | OAHFA | 0.8678 | 0.7842 | 0.9515 |
| AcHexSiE | DLCL | FA | 0.8674 | 0.7969 | 0.9380 |
| Glycocholic acid | Hex2Cer | LysoPS | 0.8670 | 0.7683 | 0.9657 |
| Cholesterol sulfate | Hex2Cer | LysoPS | 0.8670 | 0.7681 | 0.9660 |
| CerP | Hex2Cer | LysoPS | 0.8670 | 0.7683 | 0.9657 |
| Glycocholic acid | DLCL | OAHFA | 0.8662 | 0.7808 | 0.9517 |
| AcHexSiE | Hex2Cer | OAHFA | 0.8659 | 0.7815 | 0.9502 |
| Taurodeoxycholic acid | AcHexSiE | Hex2Cer | 0.8655 | 0.7850 | 0.9459 |
| CerP | FA | LysoPS | 0.8655 | 0.7728 | 0.9581 |
| LysoPC(18:2) | Hex2Cer | OAHFA | 0.8647 | 0.7799 | 0.9494 |
| LysoPC(18:2) | Cholesterol sulfate | OAHFA | 0.8639 | 0.7784 | 0.9494 |
| Glycocholic acid | GD1a | OAHFA | 0.8639 | 0.7815 | 0.9463 |
| Cholesterol sulfate | DLCL | OAHFA | 0.8635 | 0.7767 | 0.9503 |
| LysoPC(18:2) | Taurodeoxycholic acid | OAHFA | 0.8631 | 0.7791 | 0.9471 |
| LysoPC(18:2) | Glycocholic acid | OAHFA | 0.8631 | 0.7773 | 0.9489 |
| LysoPC(18:2) | Taurocholic acid | OAHFA | 0.8631 | 0.7777 | 0.9485 |
| LysoPC(18:2) | AcHexSiE | PG | 0.8631 | 0.7796 | 0.9466 |
| LysoPC(18:2) | DLCL | FA | 0.8627 | 0.7758 | 0.9496 |
| Cholesterol sulfate | GD1a | OAHFA | 0.8627 | 0.7798 | 0.9456 |
| Glycocholic acid | Taurocholic acid | LysoPS | 0.8623 | 0.7653 | 0.9593 |
| Cholesterol sulfate | Taurocholic acid | LysoPS | 0.8623 | 0.7653 | 0.9593 |
| Cholesterol sulfate | GD1a | LysoPS | 0.8623 | 0.7746 | 0.9500 |
| Taurodeoxycholic acid | AcHexSiE | FA | 0.8615 | 0.7778 | 0.9453 |
| LysoPC(18:2) | DLCL | GD1a | 0.8611 | 0.7713 | 0.9510 |
| LysoPC(18:2) | CerP | OAHFA | 0.8607 | 0.7738 | 0.9477 |
| LysoPC(18:2) | CerPE | OAHFA | 0.8607 | 0.7741 | 0.9473 |
| Glycocholic acid | GD1a | LysoPS | 0.8607 | 0.7724 | 0.9491 |
| AcHexSiE | CerPE | OAHFA | 0.8600 | 0.7809 | 0.9390 |
| AcHexSiE | CerP | OAHFA | 0.8596 | 0.7808 | 0.9383 |
| CerP | Hex1Cer | LysoPS | 0.8596 | 0.7615 | 0.9577 |
| FA | OAHFA | PG | 0.8592 | 0.7754 | 0.9429 |
| LysoPC(18:2) | Hex1Cer | OAHFA | 0.8584 | 0.7701 | 0.9466 |
| Glycocholic acid | CL | OAHFA | 0.8584 | 0.7695 | 0.9472 |
| FA | Hex1Cer | OAHFA | 0.8584 | 0.7695 | 0.9472 |
| Cholesterol sulfate | FA | OAHFA | 0.8572 | 0.7694 | 0.9450 |
| AcHexSiE | Hex1Cer | OAHFA | 0.8572 | 0.7679 | 0.9465 |
| DLCL | GD1a | OAHFA | 0.8564 | 0.7772 | 0.9356 |
| Taurodeoxycholic acid | GD1a | OAHFA | 0.8560 | 0.7691 | 0.9429 |
| Cholesterol sulfate | FA | LysoPS | 0.8560 | 0.7591 | 0.9529 |
| GD1a | OAHFA | PG | 0.8560 | 0.7703 | 0.9418 |
| Glycocholic acid | FA | OAHFA | 0.8552 | 0.7669 | 0.9435 |
| Glycocholic acid | Hex1Cer | LysoPS | 0.8552 | 0.7529 | 0.9576 |
| Cholesterol sulfate | CL | OAHFA | 0.8552 | 0.7651 | 0.9454 |
| AcHexSiE | FA | Hex2Cer | 0.8552 | 0.7695 | 0.9410 |
| LysoPC(18:2) | DLCL | PG | 0.8544 | 0.7589 | 0.9500 |
| LysoPC(18:2) | Hex2Cer | PG | 0.8544 | 0.7621 | 0.9468 |
| CL | OAHFA | PG | 0.8541 | 0.7635 | 0.9446 |
| Taurodeoxycholic acid | FA | OAHFA | 0.8537 | 0.7676 | 0.9397 |
| Cholesterol sulfate | AcHexSiE | PG | 0.8537 | 0.7659 | 0.9414 |
| Taurodeoxycholic acid | AcHexSiE | CerPE | 0.8533 | 0.7678 | 0.9387 |
| Taurodeoxycholic acid | Glycocholic acid | AcHexSiE | 0.8529 | 0.7679 | 0.9378 |
| Taurodeoxycholic acid | Cholesterol sulfate | AcHexSiE | 0.8529 | 0.7679 | 0.9378 |
| Taurodeoxycholic acid | AcHexSiE | GD1a | 0.8529 | 0.7676 | 0.9381 |
| Taurocholic acid | AcHexSiE | PG | 0.8529 | 0.7642 | 0.9416 |
| Taurodeoxycholic acid | AcHexSiE | CerP | 0.8525 | 0.7669 | 0.9380 |
| DLCL | OAHFA | PG | 0.8521 | 0.7599 | 0.9442 |
| LysoPC(18:2) | AcHexSiE | Hex1Cer | 0.8513 | 0.7619 | 0.9407 |
| Cholesterol sulfate | Hex1Cer | LysoPS | 0.8513 | 0.7464 | 0.9561 |
| Glycocholic acid | AcHexSiE | PG | 0.8505 | 0.7611 | 0.9399 |
| LysoPC(18:2) | CL | DLCL | 0.8501 | 0.7564 | 0.9439 |
| Cholesterol sulfate | DLCL | FA | 0.8501 | 0.7609 | 0.9393 |
| CerPE | FA | OAHFA | 0.8501 | 0.7672 | 0.9331 |
| LysoPC(18:2) | Hex1Cer | PG | 0.8497 | 0.7539 | 0.9455 |
| Taurocholic acid | GD1a | OAHFA | 0.8493 | 0.7606 | 0.9381 |
| DLCL | FA | GD1a | 0.8493 | 0.7648 | 0.9338 |
| Taurocholic acid | FA | OAHFA | 0.8489 | 0.7614 | 0.9365 |
| DLCL | FA | Hex1Cer | 0.8489 | 0.7597 | 0.9381 |
| Glycocholic acid | DLCL | FA | 0.8482 | 0.7584 | 0.9379 |
| Cholesterol sulfate | AcHexSiE | Hex2Cer | 0.8482 | 0.7629 | 0.9334 |
| Taurodeoxycholic acid | DLCL | OAHFA | 0.8474 | 0.7517 | 0.9430 |
| Taurodeoxycholic acid | AcHexSiE | Hex1Cer | 0.8466 | 0.7596 | 0.9336 |
| Taurocholic acid | AcHexSiE | FA | 0.8466 | 0.7542 | 0.9389 |
| DLCL | FA | Hex2Cer | 0.8466 | 0.7546 | 0.9386 |
| Cholesterol sulfate | AcHexSiE | FA | 0.8462 | 0.7533 | 0.9391 |
| LysoPC(18:2) | DLCL | Hex2Cer | 0.8458 | 0.7512 | 0.9404 |
| LysoPC(18:2) | GD1a | PG | 0.8458 | 0.7502 | 0.9414 |
| Glycocholic acid | AcHexSiE | Hex2Cer | 0.8458 | 0.7596 | 0.9319 |
| CerPE | DLCL | FA | 0.8458 | 0.7629 | 0.9287 |
| Cholesterol sulfate | CerP | LysoPS | 0.8450 | 0.7420 | 0.9480 |
| AcHexSiE | CL | PG | 0.8450 | 0.7596 | 0.9304 |
| CL | DLCL | OAHFA | 0.8446 | 0.7523 | 0.9370 |
| Glycocholic acid | CerP | LysoPS | 0.8442 | 0.7405 | 0.9479 |
| Taurocholic acid | DLCL | OAHFA | 0.8430 | 0.7459 | 0.9401 |
| Glycocholic acid | AcHexSiE | FA | 0.8419 | 0.7480 | 0.9357 |
| LysoPC(18:2) | Cholesterol sulfate | AcHexSiE | 0.8403 | 0.7492 | 0.9313 |
| LysoPC(18:2) | CL | Hex1Cer | 0.8403 | 0.7418 | 0.9387 |
| Glycocholic acid | Cholesterol sulfate | OAHFA | 0.8403 | 0.7493 | 0.9313 |
| CerP | FA | OAHFA | 0.8403 | 0.7527 | 0.9279 |
| Taurodeoxycholic acid | DLCL | FA | 0.8395 | 0.7481 | 0.9309 |
| LysoPC(18:2) | DLCL | Hex1Cer | 0.8387 | 0.7413 | 0.9361 |
| LysoPC(18:2) | FA | Hex2Cer | 0.8387 | 0.7460 | 0.9314 |
| LysoPC(18:2) | Glycocholic acid | AcHexSiE | 0.8379 | 0.7459 | 0.9299 |
| LysoPC(18:2) | AcHexSiE | CerPE | 0.8371 | 0.7464 | 0.9279 |
| Taurodeoxycholic acid | CL | OAHFA | 0.8371 | 0.7365 | 0.9378 |
| Taurocholic acid | DLCL | FA | 0.8371 | 0.7445 | 0.9298 |
| AcHexSiE | DLCL | PG | 0.8371 | 0.7504 | 0.9239 |
| LysoPC(18:2) | CL | GD1a | 0.8367 | 0.7416 | 0.9319 |
| LysoPC(18:2) | Taurocholic acid | AcHexSiE | 0.8356 | 0.7444 | 0.9267 |
| Taurodeoxycholic acid | Taurocholic acid | DLCL | 0.8356 | 0.7445 | 0.9266 |
| LysoPC(18:2) | CL | PG | 0.8348 | 0.7344 | 0.9352 |
| CerP | GD1a | OAHFA | 0.8344 | 0.7479 | 0.9209 |
| LysoPC(18:2) | CerPE | DLCL | 0.8340 | 0.7350 | 0.9330 |
| Taurocholic acid | CL | OAHFA | 0.8336 | 0.7319 | 0.9353 |
| LysoPC(18:2) | GD1a | Hex2Cer | 0.8332 | 0.7362 | 0.9302 |
| Glycocholic acid | OAHFA | PG | 0.8332 | 0.7359 | 0.9305 |
| CerPE | CL | OAHFA | 0.8332 | 0.7357 | 0.9308 |
| Cholesterol sulfate | AcHexSiE | CerPE | 0.8328 | 0.7384 | 0.9272 |
| LysoPC(18:2) | Taurodeoxycholic acid | DLCL | 0.8324 | 0.7338 | 0.9310 |
| LysoPC(18:2) | CerP | DLCL | 0.8324 | 0.7340 | 0.9309 |
| Taurocholic acid | AcHexSiE | Hex2Cer | 0.8324 | 0.7418 | 0.9230 |
| AcHexSiE | CL | FA | 0.8320 | 0.7428 | 0.9212 |
| Glycocholic acid | AcHexSiE | CerPE | 0.8316 | 0.7361 | 0.9271 |
| CerP | CL | OAHFA | 0.8312 | 0.7341 | 0.9283 |
| LysoPC(18:2) | Glycocholic acid | DLCL | 0.8308 | 0.7312 | 0.9305 |
| LysoPC(18:2) | Cholesterol sulfate | DLCL | 0.8308 | 0.7313 | 0.9304 |
| Taurodeoxycholic acid | DLCL | PG | 0.8308 | 0.7338 | 0.9279 |
| Taurodeoxycholic acid | OAHFA | PG | 0.8308 | 0.7327 | 0.9290 |
| Glycocholic acid | AcHexSiE | Hex1Cer | 0.8308 | 0.7395 | 0.9222 |
| Glycocholic acid | CerPE | OAHFA | 0.8308 | 0.7349 | 0.9268 |
| Cholesterol sulfate | OAHFA | PG | 0.8308 | 0.7330 | 0.9287 |
| LysoPC(18:2) | Taurocholic acid | DLCL | 0.8301 | 0.7308 | 0.9293 |
| Cholesterol sulfate | AcHexSiE | Hex1Cer | 0.8301 | 0.7386 | 0.9215 |
| CerPE | GD1a | OAHFA | 0.8301 | 0.7416 | 0.9186 |
| Taurodeoxycholic acid | CL | PG | 0.8293 | 0.7311 | 0.9274 |
| Glycocholic acid | FA | Hex2Cer | 0.8293 | 0.7351 | 0.9235 |
| DLCL | Hex2Cer | OAHFA | 0.8289 | 0.7241 | 0.9336 |
| Glycocholic acid | CerP | OAHFA | 0.8285 | 0.7324 | 0.9245 |
| CL | DLCL | FA | 0.8285 | 0.7376 | 0.9194 |
| FA | Hex2Cer | PG | 0.8285 | 0.7316 | 0.9253 |
| Cholesterol sulfate | CerPE | OAHFA | 0.8281 | 0.7309 | 0.9253 |
| Cholesterol sulfate | FA | Hex2Cer | 0.8277 | 0.7329 | 0.9225 |
| CL | DLCL | PG | 0.8277 | 0.7321 | 0.9233 |
| Glycocholic acid | CL | DLCL | 0.8273 | 0.7309 | 0.9237 |
| GD1a | Hex2Cer | OAHFA | 0.8273 | 0.7288 | 0.9258 |
| Taurodeoxycholic acid | GD1a | PG | 0.8269 | 0.7266 | 0.9273 |
| AcHexSiE | FA | Hex1Cer | 0.8269 | 0.7314 | 0.9224 |
| Cholesterol sulfate | CerP | OAHFA | 0.8265 | 0.7297 | 0.9233 |
| CL | LysoPS | PE(20:0p/20:3) | 0.8265 | 0.7395 | 0.9135 |
| Cholesterol sulfate | Taurocholic acid | AcHexSiE | 0.8261 | 0.7305 | 0.9218 |
| Cholesterol sulfate | CL | DLCL | 0.8261 | 0.7300 | 0.9222 |
| Cholesterol sulfate | DLCL | PG | 0.8261 | 0.7287 | 0.9236 |
| Glycocholic acid | Cholesterol sulfate | AcHexSiE | 0.8253 | 0.7287 | 0.9220 |
| Glycocholic acid | DLCL | PG | 0.8253 | 0.7274 | 0.9233 |
| GD1a | Hex1Cer | OAHFA | 0.8249 | 0.7254 | 0.9245 |
| Glycocholic acid | Taurocholic acid | AcHexSiE | 0.8245 | 0.7283 | 0.9208 |
| CL | FA | PG | 0.8245 | 0.7328 | 0.9163 |
| Cholesterol sulfate | AcHexSiE | CerP | 0.8242 | 0.7280 | 0.9203 |
| Taurocholic acid | AcHexSiE | GD1a | 0.8242 | 0.7276 | 0.9207 |
| Glycocholic acid | AcHexSiE | CerP | 0.8238 | 0.7276 | 0.9199 |
| FA | Hex1Cer | PG | 0.8238 | 0.7247 | 0.9228 |
| LysoPC(18:2) | CL | Hex2Cer | 0.8230 | 0.7229 | 0.9230 |
| LysoPC(18:2) | GD1a | Hex1Cer | 0.8230 | 0.7194 | 0.9266 |
| CerPE | DLCL | OAHFA | 0.8230 | 0.7256 | 0.9204 |
| CerPE | OAHFA | PG | 0.8230 | 0.7239 | 0.9220 |
| Cholesterol sulfate | CL | PG | 0.8222 | 0.7234 | 0.9210 |
| Cholesterol sulfate | AcHexSiE | GD1a | 0.8218 | 0.7253 | 0.9183 |
| Glycocholic acid | CL | PG | 0.8214 | 0.7218 | 0.9210 |
| Taurocholic acid | AcHexSiE | CerP | 0.8210 | 0.7241 | 0.9179 |
| Taurodeoxycholic acid | Cholesterol sulfate | OAHFA | 0.8206 | 0.7214 | 0.9198 |
| Taurodeoxycholic acid | CerPE | OAHFA | 0.8206 | 0.7204 | 0.9209 |
| Glycocholic acid | CL | GD1a | 0.8206 | 0.7224 | 0.9189 |
| AcHexSiE | DLCL | Hex1Cer | 0.8202 | 0.7241 | 0.9163 |
| LysoPC(18:2) | Taurodeoxycholic acid | CL | 0.8198 | 0.7215 | 0.9181 |
| Taurodeoxycholic acid | Glycocholic acid | OAHFA | 0.8198 | 0.7209 | 0.9188 |
| AcHexSiE | CerP | CL | 0.8198 | 0.7316 | 0.9080 |
| AcHexSiE | CL | DLCL | 0.8198 | 0.7351 | 0.9046 |
| Taurodeoxycholic acid | CL | FA | 0.8190 | 0.7236 | 0.9145 |
| Glycocholic acid | AcHexSiE | GD1a | 0.8190 | 0.7217 | 0.9163 |
| CerP | OAHFA | PG | 0.8190 | 0.7193 | 0.9188 |
| Hex1Cer | OAHFA | PG | 0.8190 | 0.7156 | 0.9225 |
| LysoPC(18:2) | AcHexSiE | FA | 0.8186 | 0.7229 | 0.9144 |
| AcHexSiE | Hex1Cer | PG | 0.8186 | 0.7212 | 0.9161 |
| LysoPC(18:2) | CerP | PG | 0.8183 | 0.7142 | 0.9223 |
| Taurodeoxycholic acid | CL | GD1a | 0.8183 | 0.7185 | 0.9180 |
| Cholesterol sulfate | CL | GD1a | 0.8183 | 0.7196 | 0.9170 |
| DLCL | FA | PG | 0.8183 | 0.7232 | 0.9133 |
| LysoPC(18:2) | Taurodeoxycholic acid | PG | 0.8175 | 0.7168 | 0.9181 |
| LysoPC(18:2) | CerP | Hex2Cer | 0.8175 | 0.7163 | 0.9187 |
| Glycocholic acid | CerP | CL | 0.8175 | 0.7194 | 0.9155 |
| Glycocholic acid | CerPE | CL | 0.8175 | 0.7166 | 0.9183 |
| Taurocholic acid | OAHFA | PG | 0.8175 | 0.7137 | 0.9212 |
| CL | ST | PC(18:1/24:2) | 0.8171 | 0.7424 | 0.8917 |
| CL | PC(18:1/24:2) | ST(d18:1/20:2) | 0.8171 | 0.7424 | 0.8917 |
| LysoPC(18:2) | Cholesterol sulfate | Hex2Cer | 0.8167 | 0.7165 | 0.9168 |
| LysoPC(18:2) | Taurocholic acid | PG | 0.8167 | 0.7138 | 0.9195 |
| LysoPC(18:2) | AcHexSiE | CerP | 0.8167 | 0.7205 | 0.9128 |
| LysoPC(18:2) | CerPE | PG | 0.8167 | 0.7113 | 0.9221 |
| Taurodeoxycholic acid | FA | Hex2Cer | 0.8167 | 0.7183 | 0.9151 |
| Taurodeoxycholic acid | Hex2Cer | OAHFA | 0.8167 | 0.7098 | 0.9235 |
| Cholesterol sulfate | CerPE | CL | 0.8167 | 0.7159 | 0.9174 |
| CL | GD1a | PG | 0.8167 | 0.7184 | 0.9150 |
| Cholesterol sulfate | DLCL | GD1a | 0.8163 | 0.7174 | 0.9152 |
| Taurocholic acid | CerPE | OAHFA | 0.8163 | 0.7131 | 0.9195 |
| LysoPC(18:2) | Taurodeoxycholic acid | Hex2Cer | 0.8159 | 0.7162 | 0.9156 |
| Taurodeoxycholic acid | Taurocholic acid | OAHFA | 0.8159 | 0.7136 | 0.9182 |
| Glycocholic acid | DLCL | GD1a | 0.8159 | 0.7167 | 0.9151 |
| Cholesterol sulfate | CerP | CL | 0.8159 | 0.7176 | 0.9142 |
| Taurocholic acid | FA | Hex2Cer | 0.8159 | 0.7176 | 0.9142 |
| LysoPC(18:2) | AcHexSiE | GD1a | 0.8155 | 0.7195 | 0.9115 |
| LysoPC(18:2) | Glycocholic acid | Hex2Cer | 0.8151 | 0.7146 | 0.9156 |
| LysoPC(18:2) | FA | Hex1Cer | 0.8151 | 0.7106 | 0.9197 |
| Taurodeoxycholic acid | CerPE | CL | 0.8151 | 0.7124 | 0.9178 |
| AcHexSiE | DLCL | Hex2Cer | 0.8151 | 0.7191 | 0.9111 |
| Taurocholic acid | AcHexSiE | CerPE | 0.8147 | 0.7161 | 0.9133 |
| Hex2Cer | OAHFA | PG | 0.8147 | 0.7096 | 0.9199 |
| LysoPC(18:2) | Cholesterol sulfate | PG | 0.8143 | 0.7095 | 0.9191 |
| LysoPC(18:2) | Taurocholic acid | Hex2Cer | 0.8143 | 0.7134 | 0.9153 |
| Glycocholic acid | Taurocholic acid | OAHFA | 0.8143 | 0.7126 | 0.9161 |
| LysoPC(18:2) | Glycocholic acid | PG | 0.8135 | 0.7085 | 0.9185 |
| LysoPC(18:2) | CerPE | Hex2Cer | 0.8135 | 0.7125 | 0.9146 |
| AcHexSiE | Hex2Cer | PG | 0.8131 | 0.7164 | 0.9099 |
| Taurodeoxycholic acid | CerP | OAHFA | 0.8127 | 0.7089 | 0.9166 |
| Cholesterol sulfate | Taurocholic acid | OAHFA | 0.8127 | 0.7101 | 0.9154 |
| LysoPC(18:2) | CerPE | CL | 0.8120 | 0.7083 | 0.9156 |
| LysoPC(18:2) | FA | PG | 0.8120 | 0.7074 | 0.9165 |
| Taurodeoxycholic acid | CL | DLCL | 0.8112 | 0.7091 | 0.9133 |
| LysoPC(18:2) | Taurocholic acid | CL | 0.8104 | 0.7088 | 0.9120 |
| LysoPC(18:2) | CerP | CL | 0.8104 | 0.7066 | 0.9142 |
| Taurodeoxycholic acid | Taurocholic acid | CL | 0.8104 | 0.7090 | 0.9118 |
| Taurocholic acid | Hex2Cer | OAHFA | 0.8104 | 0.7026 | 0.9182 |
| Taurodeoxycholic acid | DLCL | GD1a | 0.8100 | 0.7090 | 0.9110 |
| CL | FA | GD1a | 0.8100 | 0.7097 | 0.9103 |
| DLCL | Hex1Cer | OAHFA | 0.8100 | 0.6960 | 0.9240 |
| LysoPC(18:2) | Hex1Cer | Hex2Cer | 0.8096 | 0.7083 | 0.9109 |
| Taurocholic acid | CL | PG | 0.8096 | 0.7045 | 0.9147 |
| CerP | CL | PG | 0.8096 | 0.7090 | 0.9102 |
| CerP | DLCL | OAHFA | 0.8096 | 0.7089 | 0.9103 |
| Glycocholic acid | CerP | DLCL | 0.8088 | 0.7098 | 0.9078 |
| Glycocholic acid | CerPE | DLCL | 0.8088 | 0.7063 | 0.9113 |
| Glycocholic acid | Hex2Cer | OAHFA | 0.8088 | 0.7045 | 0.9131 |
| Cholesterol sulfate | Hex2Cer | OAHFA | 0.8088 | 0.7040 | 0.9136 |
| Taurocholic acid | DLCL | PG | 0.8080 | 0.7031 | 0.9130 |
| Cholesterol sulfate | CerP | DLCL | 0.8076 | 0.7080 | 0.9072 |
| Taurodeoxycholic acid | Hex1Cer | OAHFA | 0.8072 | 0.6972 | 0.9172 |
| FA | GD1a | Hex2Cer | 0.8072 | 0.7041 | 0.9104 |
| Taurodeoxycholic acid | Glycocholic acid | CL | 0.8065 | 0.7052 | 0.9077 |
| CerPE | Hex2Cer | OAHFA | 0.8065 | 0.6963 | 0.9167 |
| DLCL | GD1a | PG | 0.8065 | 0.7065 | 0.9064 |
| Cholesterol sulfate | CerPE | DLCL | 0.8061 | 0.7025 | 0.9096 |
| Taurocholic acid | CerP | OAHFA | 0.8061 | 0.7005 | 0.9116 |
| Taurocholic acid | CL | GD1a | 0.8061 | 0.7035 | 0.9086 |
| LysoPC(18:2) | CerPE | GD1a | 0.8057 | 0.7024 | 0.9089 |
| Taurodeoxycholic acid | CerPE | DLCL | 0.8053 | 0.7023 | 0.9082 |
| Taurodeoxycholic acid | Cholesterol sulfate | CL | 0.8049 | 0.7027 | 0.9070 |
| Taurodeoxycholic acid | Hex1Cer | PG | 0.8049 | 0.6968 | 0.9130 |
| Taurocholic acid | CL | DLCL | 0.8045 | 0.7005 | 0.9085 |
| Taurodeoxycholic acid | Cholesterol sulfate | DLCL | 0.8041 | 0.7041 | 0.9041 |
| AcHexSiE | CL | Hex1Cer | 0.8037 | 0.7036 | 0.9038 |
| CerP | CL | GD1a | 0.8037 | 0.7069 | 0.9005 |
| LysoPC(18:2) | Glycocholic acid | CL | 0.8033 | 0.6983 | 0.9083 |
| LysoPC(18:2) | CerPE | Hex1Cer | 0.8033 | 0.6978 | 0.9088 |
| Glycocholic acid | Hex1Cer | OAHFA | 0.8033 | 0.6956 | 0.9110 |
| Taurocholic acid | AcHexSiE | Hex1Cer | 0.8033 | 0.7038 | 0.9028 |
| CerPE | Hex1Cer | OAHFA | 0.8033 | 0.6941 | 0.9125 |
| Taurodeoxycholic acid | FA | Hex1Cer | 0.8025 | 0.6977 | 0.9073 |
| LysoPC(18:2) | CL | FA | 0.8021 | 0.6978 | 0.9064 |
| LysoPC(18:2) | Cholesterol sulfate | CL | 0.8017 | 0.6967 | 0.9068 |
| Taurodeoxycholic acid | Glycocholic acid | DLCL | 0.8017 | 0.7011 | 0.9024 |
| Taurodeoxycholic acid | CerP | CL | 0.8017 | 0.6977 | 0.9058 |
| Glycocholic acid | CL | FA | 0.8017 | 0.6974 | 0.9061 |
| AcHexSiE | FA | PG | 0.8017 | 0.7031 | 0.9003 |
| Taurodeoxycholic acid | CerP | DLCL | 0.8009 | 0.6986 | 0.9033 |
| Cholesterol sulfate | CL | FA | 0.8009 | 0.6964 | 0.9055 |
| CL | DLCL | GD1a | 0.8006 | 0.7061 | 0.8950 |
| Taurodeoxycholic acid | Hex2Cer | PG | 0.8002 | 0.6925 | 0.9078 |
| AcHexSiE | CerPE | CL | 0.8002 | 0.7054 | 0.8949 |
| Cholesterol sulfate | Hex1Cer | OAHFA | 0.7994 | 0.6905 | 0.9083 |
| AcHexSiE | CerP | PG | 0.7994 | 0.7010 | 0.8977 |
| AcHexSiE | CL | GD1a | 0.7994 | 0.7086 | 0.8901 |
| CL | FA | Hex1Cer | 0.7994 | 0.6958 | 0.9029 |
| CL | Hex1Cer | OAHFA | 0.7994 | 0.6827 | 0.9161 |
| Taurodeoxycholic acid | Taurocholic acid | PG | 0.7986 | 0.6939 | 0.9033 |
| Taurocholic acid | DLCL | GD1a | 0.7982 | 0.6942 | 0.9022 |
| FA | GD1a | PG | 0.7982 | 0.6963 | 0.9000 |
| LysoPC(18:2) | Taurodeoxycholic acid | Hex1Cer | 0.7978 | 0.6917 | 0.9039 |
| Taurodeoxycholic acid | FA | PG | 0.7978 | 0.6955 | 0.9001 |
| AcHexSiE | GD1a | PG | 0.7978 | 0.7010 | 0.8946 |
| CL | Hex1Cer | PG | 0.7974 | 0.6904 | 0.9044 |
| Taurocholic acid | CL | FA | 0.7970 | 0.6925 | 0.9015 |
| Taurocholic acid | Hex1Cer | OAHFA | 0.7970 | 0.6846 | 0.9094 |
| AcHexSiE | CL | Hex2Cer | 0.7970 | 0.6960 | 0.8981 |
| Glycocholic acid | Taurocholic acid | DLCL | 0.7962 | 0.6935 | 0.8989 |
| CL | Hex2Cer | OAHFA | 0.7958 | 0.6787 | 0.9130 |
| LysoPC(18:2) | Taurodeoxycholic acid | GD1a | 0.7954 | 0.6897 | 0.9011 |
| Cholesterol sulfate | GD1a | PG | 0.7954 | 0.6898 | 0.9011 |
| DLCL | Hex1Cer | PG | 0.7954 | 0.6881 | 0.9027 |
| FA | GD1a | Hex1Cer | 0.7954 | 0.6895 | 0.9014 |
| Cholesterol sulfate | CerPE | FA | 0.7950 | 0.6867 | 0.9034 |
| LysoPC(18:2) | Glycocholic acid | Hex1Cer | 0.7946 | 0.6877 | 0.9016 |
| LysoPC(18:2) | Cholesterol sulfate | Hex1Cer | 0.7946 | 0.6876 | 0.9017 |
| LysoPC(18:2) | Taurocholic acid | Hex1Cer | 0.7946 | 0.6882 | 0.9011 |
| Glycocholic acid | CerPE | FA | 0.7946 | 0.6862 | 0.9031 |
| Glycocholic acid | Hex1Cer | PG | 0.7946 | 0.6884 | 0.9009 |
| AcHexSiE | CerP | FA | 0.7946 | 0.6978 | 0.8915 |
| AcHexSiE | FA | GD1a | 0.7946 | 0.6984 | 0.8909 |
| Taurocholic acid | CerPE | CL | 0.7943 | 0.6860 | 0.9025 |
| Hex1Cer | Hex2Cer | OAHFA | 0.7939 | 0.6787 | 0.9090 |
| LysoPC(18:2) | CerP | Hex1Cer | 0.7931 | 0.6853 | 0.9009 |
| Glycocholic acid | GD1a | PG | 0.7931 | 0.6869 | 0.8992 |
| Taurocholic acid | FA | Hex1Cer | 0.7931 | 0.6887 | 0.8974 |
| Glycocholic acid | FA | Hex1Cer | 0.7923 | 0.6854 | 0.8991 |
| CL | FA | Hex2Cer | 0.7923 | 0.6846 | 0.9000 |
| Taurodeoxycholic acid | CL | Hex1Cer | 0.7915 | 0.6819 | 0.9012 |
| Taurocholic acid | Hex1Cer | PG | 0.7915 | 0.6807 | 0.9023 |
| CerP | CerPE | OAHFA | 0.7915 | 0.6860 | 0.8970 |
| CerPE | FA | Hex2Cer | 0.7915 | 0.6840 | 0.8990 |
| Taurodeoxycholic acid | CerP | PG | 0.7907 | 0.6839 | 0.8975 |
| Cholesterol sulfate | Taurocholic acid | DLCL | 0.7907 | 0.6861 | 0.8953 |
| Cholesterol sulfate | Hex1Cer | PG | 0.7907 | 0.6837 | 0.8977 |
| LysoPC(18:2) | Glycocholic acid | CerPE | 0.7899 | 0.6818 | 0.8981 |
| LysoPC(18:2) | Taurocholic acid | CerPE | 0.7899 | 0.6828 | 0.8970 |
| Taurodeoxycholic acid | Taurocholic acid | GD1a | 0.7899 | 0.6851 | 0.8948 |
| Cholesterol sulfate | FA | GD1a | 0.7895 | 0.6774 | 0.9016 |
| Taurodeoxycholic acid | Cholesterol sulfate | PG | 0.7891 | 0.6826 | 0.8957 |
| Cholesterol sulfate | FA | Hex1Cer | 0.7891 | 0.6819 | 0.8964 |
| Taurocholic acid | CerP | CL | 0.7887 | 0.6818 | 0.8957 |
| AcHexSiE | CerPE | DLCL | 0.7884 | 0.6994 | 0.8773 |
| CerP | Hex2Cer | OAHFA | 0.7880 | 0.6715 | 0.9044 |
| Taurocholic acid | GD1a | PG | 0.7876 | 0.6786 | 0.8965 |
| CerP | Hex1Cer | OAHFA | 0.7872 | 0.6715 | 0.9028 |
| LysoPC(18:2) | Taurodeoxycholic acid | CerPE | 0.7868 | 0.6801 | 0.8934 |
| Taurodeoxycholic acid | Glycocholic acid | PG | 0.7860 | 0.6786 | 0.8933 |
| Taurodeoxycholic acid | CerPE | FA | 0.7860 | 0.6815 | 0.8905 |
| Glycocholic acid | FA | GD1a | 0.7860 | 0.6732 | 0.8988 |
| CerP | DLCL | FA | 0.7860 | 0.6903 | 0.8817 |
| LysoPC(18:2) | Cholesterol sulfate | CerPE | 0.7852 | 0.6757 | 0.8947 |
| Taurocholic acid | Hex2Cer | PG | 0.7852 | 0.6733 | 0.8972 |
| CerP | DLCL | PG | 0.7852 | 0.6768 | 0.8937 |
| CerPE | CL | FA | 0.7852 | 0.6787 | 0.8917 |
| Taurodeoxycholic acid | CL | Hex2Cer | 0.7844 | 0.6766 | 0.8923 |
| Taurocholic acid | FA | PG | 0.7844 | 0.6790 | 0.8898 |
| CL | Hex2Cer | PG | 0.7840 | 0.6729 | 0.8951 |
| CerPE | FA | Hex1Cer | 0.7836 | 0.6757 | 0.8916 |
| AcHexSiE | CerPE | Hex2Cer | 0.7832 | 0.6795 | 0.8870 |
| GD1a | Hex1Cer | PG | 0.7832 | 0.6748 | 0.8916 |
| Glycocholic acid | Taurocholic acid | CL | 0.7828 | 0.6729 | 0.8928 |
| Taurocholic acid | CerP | DLCL | 0.7825 | 0.6754 | 0.8895 |
| AcHexSiE | CerPE | FA | 0.7825 | 0.6839 | 0.8810 |
| Taurodeoxycholic acid | DLCL | Hex1Cer | 0.7821 | 0.6696 | 0.8945 |
| Taurodeoxycholic acid | DLCL | Hex2Cer | 0.7821 | 0.6725 | 0.8916 |
| Glycocholic acid | Hex2Cer | PG | 0.7821 | 0.6730 | 0.8911 |
| AcHexSiE | Hex1Cer | Hex2Cer | 0.7821 | 0.6780 | 0.8861 |
| Cholesterol sulfate | CerP | PG | 0.7813 | 0.6737 | 0.8889 |
| Cholesterol sulfate | Hex2Cer | PG | 0.7813 | 0.6723 | 0.8902 |
| AcHexSiE | GD1a | Hex2Cer | 0.7813 | 0.6766 | 0.8859 |
| Glycocholic acid | CerP | PG | 0.7805 | 0.6724 | 0.8885 |
| CerPE | CL | GD1a | 0.7805 | 0.6778 | 0.8831 |
| Taurocholic acid | CerPE | DLCL | 0.7801 | 0.6708 | 0.8894 |
| LysoPC(18:2) | Taurocholic acid | GD1a | 0.7797 | 0.6712 | 0.8882 |
| Taurodeoxycholic acid | CerPE | PG | 0.7797 | 0.6690 | 0.8904 |
| Cholesterol sulfate | Taurocholic acid | CL | 0.7797 | 0.6687 | 0.8907 |
| LysoPC(18:2) | CerPE | FA | 0.7793 | 0.6679 | 0.8907 |
| AcHexSiE | CerP | Hex2Cer | 0.7789 | 0.6738 | 0.8840 |
| AcHexSiE | CerPE | Hex1Cer | 0.7789 | 0.6729 | 0.8849 |
| CerP | FA | GD1a | 0.7789 | 0.6735 | 0.8843 |
| LysoPC(18:2) | CerP | CerPE | 0.7785 | 0.6683 | 0.8888 |
| LysoPC(18:2) | CerP | GD1a | 0.7773 | 0.6689 | 0.8858 |
| Taurodeoxycholic acid | FA | GD1a | 0.7773 | 0.6671 | 0.8876 |
| CerP | FA | Hex2Cer | 0.7773 | 0.6670 | 0.8876 |
| FA | Hex1Cer | Hex2Cer | 0.7766 | 0.6663 | 0.8868 |
| Glycocholic acid | Cholesterol sulfate | PG | 0.7758 | 0.6663 | 0.8852 |
| LysoPC(18:2) | FA | GD1a | 0.7754 | 0.6672 | 0.8835 |
| Cholesterol sulfate | FA | PG | 0.7750 | 0.6676 | 0.8823 |
| CerP | Hex1Cer | PG | 0.7750 | 0.6633 | 0.8867 |
| AcHexSiE | CerPE | PG | 0.7746 | 0.6708 | 0.8784 |
| Taurodeoxycholic acid | CerPE | Hex2Cer | 0.7742 | 0.6634 | 0.8850 |
| Glycocholic acid | FA | PG | 0.7742 | 0.6665 | 0.8818 |
| AcHexSiE | GD1a | Hex1Cer | 0.7742 | 0.6668 | 0.8816 |
| CerP | FA | Hex1Cer | 0.7742 | 0.6650 | 0.8834 |
| AcHexSiE | CerP | Hex1Cer | 0.7738 | 0.6669 | 0.8807 |
| DLCL | GD1a | Hex1Cer | 0.7738 | 0.6628 | 0.8848 |
| LysoPC(18:2) | Cholesterol sulfate | GD1a | 0.7734 | 0.6637 | 0.8831 |
| CerPE | Hex1Cer | PG | 0.7734 | 0.6612 | 0.8857 |
| DLCL | Hex2Cer | PG | 0.7730 | 0.6599 | 0.8861 |
| GD1a | Hex2Cer | PG | 0.7730 | 0.6614 | 0.8846 |
| LysoPC(18:2) | Glycocholic acid | GD1a | 0.7726 | 0.6628 | 0.8825 |
| Taurodeoxycholic acid | GD1a | Hex2Cer | 0.7726 | 0.6620 | 0.8832 |
| Glycocholic acid | Taurocholic acid | PG | 0.7726 | 0.6613 | 0.8839 |
| Glycocholic acid | CerPE | Hex1Cer | 0.7726 | 0.6618 | 0.8835 |
| Cholesterol sulfate | Taurocholic acid | PG | 0.7726 | 0.6613 | 0.8839 |
| Cholesterol sulfate | CerP | GD1a | 0.7714 | 0.6607 | 0.8822 |
| Glycocholic acid | DLCL | Hex1Cer | 0.7710 | 0.6575 | 0.8846 |
| CerPE | CL | PG | 0.7710 | 0.6610 | 0.8811 |
| Hex1Cer | Hex2Cer | PG | 0.7710 | 0.6557 | 0.8864 |
| Cholesterol sulfate | CerPE | GD1a | 0.7707 | 0.6586 | 0.8827 |
| Glycocholic acid | CerPE | GD1a | 0.7703 | 0.6581 | 0.8824 |
| CerP | FA | PG | 0.7703 | 0.6644 | 0.8761 |
| Glycocholic acid | CerP | GD1a | 0.7695 | 0.6583 | 0.8806 |
| Cholesterol sulfate | DLCL | Hex1Cer | 0.7687 | 0.6542 | 0.8832 |
| CerP | GD1a | PG | 0.7687 | 0.6606 | 0.8768 |
| Taurodeoxycholic acid | CerPE | GD1a | 0.7683 | 0.6577 | 0.8789 |
| CL | DLCL | PE(20:0p/20:3) | 0.7683 | 0.6910 | 0.8456 |
| Taurocholic acid | CerPE | FA | 0.7679 | 0.6582 | 0.8776 |
| CerPE | DLCL | PG | 0.7679 | 0.6571 | 0.8787 |
| Taurocholic acid | DLCL | Hex1Cer | 0.7671 | 0.6504 | 0.8838 |
| CerP | CL | FA | 0.7663 | 0.6563 | 0.8764 |
| CerPE | CL | DLCL | 0.7651 | 0.6564 | 0.8739 |
| Cholesterol sulfate | CerPE | Hex1Cer | 0.7648 | 0.6525 | 0.8770 |
| Taurodeoxycholic acid | Glycocholic acid | Taurocholic acid | 0.7640 | 0.6545 | 0.8734 |
| Taurocholic acid | CerP | PG | 0.7640 | 0.6507 | 0.8773 |
| CerPE | Hex2Cer | PG | 0.7640 | 0.6485 | 0.8795 |
| Glycocholic acid | CerP | CerPE | 0.7632 | 0.6498 | 0.8766 |
| CerPE | DLCL | Hex1Cer | 0.7632 | 0.6484 | 0.8779 |
| Taurodeoxycholic acid | Cholesterol sulfate | Taurocholic acid | 0.7624 | 0.6523 | 0.8725 |
| Cholesterol sulfate | CerP | CerPE | 0.7620 | 0.6476 | 0.8764 |
| Glycocholic acid | CL | Hex1Cer | 0.7616 | 0.6457 | 0.8775 |
| CerPE | DLCL | GD1a | 0.7616 | 0.6623 | 0.8609 |
| CerP | DLCL | Hex1Cer | 0.7612 | 0.6436 | 0.8788 |
| Taurodeoxycholic acid | CerP | FA | 0.7608 | 0.6461 | 0.8755 |
| AcHexSiE | DLCL | GD1a | 0.7608 | 0.6743 | 0.8474 |
| CerP | CL | DLCL | 0.7608 | 0.6550 | 0.8666 |
| CL | DLCL | Hex1Cer | 0.7600 | 0.6410 | 0.8790 |
| Glycocholic acid | Cholesterol sulfate | DLCL | 0.7585 | 0.6404 | 0.8766 |
| Glycocholic acid | CerP | FA | 0.7585 | 0.6432 | 0.8737 |
| Cholesterol sulfate | CerPE | PG | 0.7585 | 0.6449 | 0.8720 |
| Cholesterol sulfate | CL | Hex1Cer | 0.7577 | 0.6412 | 0.8742 |
| Glycocholic acid | CerPE | PG | 0.7569 | 0.6428 | 0.8710 |
| Taurocholic acid | CerPE | PG | 0.7569 | 0.6406 | 0.8732 |
| Taurocholic acid | CL | Hex1Cer | 0.7569 | 0.6392 | 0.8746 |
| Taurocholic acid | DLCL | Hex2Cer | 0.7569 | 0.6418 | 0.8720 |
| DLCL | GD1a | Hex2Cer | 0.7569 | 0.6434 | 0.8704 |
| Cholesterol sulfate | CerP | FA | 0.7565 | 0.6407 | 0.8723 |
| Taurocholic acid | FA | GD1a | 0.7561 | 0.6402 | 0.8720 |
| CerPE | CL | Hex1Cer | 0.7561 | 0.6407 | 0.8715 |
| CL | GD1a | Hex1Cer | 0.7557 | 0.6414 | 0.8700 |
| Taurodeoxycholic acid | Cholesterol sulfate | CerPE | 0.7553 | 0.6423 | 0.8683 |
| Taurodeoxycholic acid | Taurocholic acid | FA | 0.7553 | 0.6399 | 0.8707 |
| Taurodeoxycholic acid | GD1a | Hex1Cer | 0.7553 | 0.6413 | 0.8693 |
| Glycocholic acid | DLCL | Hex2Cer | 0.7553 | 0.6415 | 0.8692 |
| Taurodeoxycholic acid | Taurocholic acid | CerPE | 0.7545 | 0.6414 | 0.8677 |
| Glycocholic acid | Cholesterol sulfate | Hex2Cer | 0.7545 | 0.6425 | 0.8666 |
| AcHexSiE | CerP | DLCL | 0.7541 | 0.6623 | 0.8459 |
| Taurodeoxycholic acid | Glycocholic acid | CerPE | 0.7537 | 0.6407 | 0.8668 |
| Cholesterol sulfate | DLCL | Hex2Cer | 0.7537 | 0.6390 | 0.8685 |
| Taurodeoxycholic acid | CerP | GD1a | 0.7533 | 0.6412 | 0.8655 |
| CerP | Hex2Cer | PG | 0.7533 | 0.6362 | 0.8705 |
| Taurodeoxycholic acid | Glycocholic acid | GD1a | 0.7530 | 0.6410 | 0.8649 |
| Taurodeoxycholic acid | Glycocholic acid | Hex2Cer | 0.7530 | 0.6404 | 0.8655 |
| Taurodeoxycholic acid | Cholesterol sulfate | Hex2Cer | 0.7530 | 0.6402 | 0.8657 |
| Taurodeoxycholic acid | Taurocholic acid | Hex2Cer | 0.7530 | 0.6388 | 0.8671 |
| Taurodeoxycholic acid | CerPE | Hex1Cer | 0.7522 | 0.6363 | 0.8680 |
| CerPE | FA | GD1a | 0.7518 | 0.6424 | 0.8611 |
| Taurodeoxycholic acid | Cholesterol sulfate | GD1a | 0.7514 | 0.6390 | 0.8637 |
| Taurodeoxycholic acid | CerP | Hex2Cer | 0.7498 | 0.6331 | 0.8665 |
| Taurodeoxycholic acid | Hex1Cer | Hex2Cer | 0.7498 | 0.6346 | 0.8650 |
| Taurocholic acid | CL | Hex2Cer | 0.7490 | 0.6331 | 0.8649 |
| DLCL | Hex1Cer | Hex2Cer | 0.7490 | 0.6277 | 0.8704 |
| CerPE | FA | PG | 0.7486 | 0.6387 | 0.8585 |
| CerPE | DLCL | Hex2Cer | 0.7470 | 0.6286 | 0.8655 |
| Taurodeoxycholic acid | CerP | CerPE | 0.7467 | 0.6315 | 0.8618 |
| AcHexSiE | CerP | CerPE | 0.7467 | 0.6459 | 0.8475 |
| AcHexSiE | CerP | GD1a | 0.7467 | 0.6561 | 0.8372 |
| Glycocholic acid | CerPE | Hex2Cer | 0.7459 | 0.6328 | 0.8589 |
| CerPE | GD1a | PG | 0.7451 | 0.6325 | 0.8577 |
| CerP | CerPE | CL | 0.7447 | 0.6317 | 0.8577 |
| Taurodeoxycholic acid | Taurocholic acid | CerP | 0.7443 | 0.6297 | 0.8588 |
| Cholesterol sulfate | CerPE | Hex2Cer | 0.7443 | 0.6303 | 0.8583 |
| Taurocholic acid | CL | ST | 0.7439 | 0.6665 | 0.8214 |
| Taurocholic acid | CL | ST(d18:1/20:2) | 0.7439 | 0.6665 | 0.8214 |
| Taurodeoxycholic acid | Glycocholic acid | FA | 0.7435 | 0.6268 | 0.8602 |
| Taurodeoxycholic acid | Taurocholic acid | Hex1Cer | 0.7427 | 0.6238 | 0.8617 |
| Glycocholic acid | CL | Hex2Cer | 0.7427 | 0.6262 | 0.8592 |
| LysoPC(18:2) | Taurodeoxycholic acid | CerP | 0.7419 | 0.6275 | 0.8563 |
| Taurodeoxycholic acid | Cholesterol sulfate | FA | 0.7419 | 0.6250 | 0.8588 |
| Glycocholic acid | Cholesterol sulfate | CL | 0.7419 | 0.6191 | 0.8647 |
| Glycocholic acid | GD1a | Hex1Cer | 0.7419 | 0.6264 | 0.8575 |
| Taurocholic acid | CerPE | GD1a | 0.7415 | 0.6258 | 0.8573 |
| Cholesterol sulfate | GD1a | Hex1Cer | 0.7411 | 0.6254 | 0.8569 |
| Glycocholic acid | Taurocholic acid | CerPE | 0.7404 | 0.6246 | 0.8561 |
| Cholesterol sulfate | CL | Hex2Cer | 0.7404 | 0.6229 | 0.8578 |
| Taurocholic acid | CerPE | Hex2Cer | 0.7404 | 0.6243 | 0.8565 |
| CerPE | CL | Hex2Cer | 0.7400 | 0.6200 | 0.8600 |
| Taurodeoxycholic acid | CerP | Hex1Cer | 0.7396 | 0.6205 | 0.8587 |
| Cholesterol sulfate | Taurocholic acid | CerPE | 0.7396 | 0.6236 | 0.8555 |
| CL | GD1a | Hex2Cer | 0.7396 | 0.6218 | 0.8574 |
| Taurodeoxycholic acid | Cholesterol sulfate | Hex1Cer | 0.7388 | 0.6198 | 0.8578 |
| Taurocholic acid | GD1a | Hex2Cer | 0.7388 | 0.6242 | 0.8534 |
| Taurodeoxycholic acid | Glycocholic acid | Hex1Cer | 0.7380 | 0.6195 | 0.8565 |
| Glycocholic acid | GD1a | Hex2Cer | 0.7380 | 0.6230 | 0.8530 |
| CerP | CL | Hex1Cer | 0.7380 | 0.6164 | 0.8596 |
| Taurodeoxycholic acid | Cholesterol sulfate | CerP | 0.7372 | 0.6219 | 0.8525 |
| Taurocholic acid | CerP | GD1a | 0.7368 | 0.6200 | 0.8536 |
| LysoPC(18:2) | Taurodeoxycholic acid | Glycocholic acid | 0.7364 | 0.6204 | 0.8525 |
| LysoPC(18:2) | Taurodeoxycholic acid | FA | 0.7364 | 0.6205 | 0.8523 |
| LysoPC(18:2) | Taurocholic acid | CerP | 0.7364 | 0.6196 | 0.8533 |
| Glycocholic acid | Cholesterol sulfate | CerPE | 0.7364 | 0.6200 | 0.8529 |
| Cholesterol sulfate | GD1a | Hex2Cer | 0.7364 | 0.6210 | 0.8518 |
| LysoPC(18:2) | Taurodeoxycholic acid | Cholesterol sulfate | 0.7349 | 0.6186 | 0.8511 |
| Glycocholic acid | Taurocholic acid | GD1a | 0.7349 | 0.6177 | 0.8520 |
| CerPE | GD1a | Hex1Cer | 0.7349 | 0.6190 | 0.8507 |
| CL | DLCL | Hex2Cer | 0.7349 | 0.6117 | 0.8580 |
| Taurodeoxycholic acid | Glycocholic acid | CerP | 0.7341 | 0.6182 | 0.8500 |
| Taurocholic acid | CerPE | Hex1Cer | 0.7333 | 0.6149 | 0.8517 |
| CerP | CerPE | PG | 0.7333 | 0.6176 | 0.8489 |
| LysoPC(18:2) | Glycocholic acid | FA | 0.7317 | 0.6128 | 0.8506 |
| Taurocholic acid | GD1a | Hex1Cer | 0.7317 | 0.6148 | 0.8486 |
| CerPE | GD1a | Hex2Cer | 0.7313 | 0.6150 | 0.8476 |
| LysoPC(18:2) | Taurodeoxycholic acid | Taurocholic acid | 0.7309 | 0.6138 | 0.8481 |
| Glycocholic acid | CerP | Hex1Cer | 0.7301 | 0.6099 | 0.8503 |
| CerP | DLCL | Hex2Cer | 0.7301 | 0.6070 | 0.8533 |
| LysoPC(18:2) | Glycocholic acid | Cholesterol sulfate | 0.7293 | 0.6100 | 0.8487 |
| LysoPC(18:2) | Glycocholic acid | CerP | 0.7293 | 0.6105 | 0.8482 |
| LysoPC(18:2) | Cholesterol sulfate | CerP | 0.7293 | 0.6105 | 0.8482 |
| Glycocholic acid | Hex1Cer | Hex2Cer | 0.7293 | 0.6106 | 0.8481 |
| Cholesterol sulfate | Taurocholic acid | GD1a | 0.7293 | 0.6116 | 0.8471 |
| LysoPC(18:2) | Cholesterol sulfate | FA | 0.7286 | 0.6092 | 0.8479 |
| Glycocholic acid | Cholesterol sulfate | Hex1Cer | 0.7278 | 0.6121 | 0.8435 |
| Cholesterol sulfate | CerP | Hex1Cer | 0.7262 | 0.6053 | 0.8471 |
| Taurocholic acid | CerP | CerPE | 0.7250 | 0.6061 | 0.8440 |
| CerP | CerPE | FA | 0.7242 | 0.6105 | 0.8379 |
| LysoPC(18:2) | Glycocholic acid | Taurocholic acid | 0.7238 | 0.6047 | 0.8430 |
| LysoPC(18:2) | Cholesterol sulfate | Taurocholic acid | 0.7238 | 0.6047 | 0.8430 |
| CL | Hex1Cer | Hex2Cer | 0.7231 | 0.5972 | 0.8489 |
| LysoPC(18:2) | Taurocholic acid | FA | 0.7223 | 0.6020 | 0.8425 |
| Glycocholic acid | Taurocholic acid | CerP | 0.7223 | 0.6036 | 0.8410 |
| Cholesterol sulfate | Taurocholic acid | CerP | 0.7223 | 0.6034 | 0.8411 |
| Glycocholic acid | Taurocholic acid | FA | 0.7207 | 0.6002 | 0.8412 |
| CerP | CerPE | DLCL | 0.7207 | 0.6134 | 0.8280 |
| CerP | CerPE | GD1a | 0.7207 | 0.6128 | 0.8286 |
| CerP | CerPE | Hex1Cer | 0.7207 | 0.6007 | 0.8407 |
| LysoPC(18:2) | CerP | FA | 0.7203 | 0.6007 | 0.8399 |
| GD1a | Hex1Cer | Hex2Cer | 0.7199 | 0.6007 | 0.8391 |
| CerP | DLCL | GD1a | 0.7183 | 0.6156 | 0.8211 |
| CerP | GD1a | Hex2Cer | 0.7183 | 0.5988 | 0.8378 |
| CerP | GD1a | Hex1Cer | 0.7179 | 0.5990 | 0.8369 |
| Glycocholic acid | CerP | Hex2Cer | 0.7175 | 0.5950 | 0.8401 |
| Cholesterol sulfate | Taurocholic acid | FA | 0.7175 | 0.5967 | 0.8384 |
| Cholesterol sulfate | Hex1Cer | Hex2Cer | 0.7175 | 0.5963 | 0.8388 |
| Taurocholic acid | CerP | Hex2Cer | 0.7168 | 0.5939 | 0.8396 |
| Glycocholic acid | Taurocholic acid | Hex2Cer | 0.7144 | 0.5942 | 0.8346 |
| Cholesterol sulfate | CerP | Hex2Cer | 0.7144 | 0.5907 | 0.8381 |
| Cholesterol sulfate | Taurocholic acid | Hex2Cer | 0.7136 | 0.5929 | 0.8343 |
| Taurocholic acid | CerP | FA | 0.7136 | 0.5929 | 0.8343 |
| Taurocholic acid | Hex1Cer | Hex2Cer | 0.7136 | 0.5914 | 0.8358 |
| Taurodeoxycholic acid | Glycocholic acid | Cholesterol sulfate | 0.7128 | 0.5923 | 0.8333 |
| CerPE | Hex1Cer | Hex2Cer | 0.7113 | 0.5878 | 0.8347 |
| Glycocholic acid | Taurocholic acid | Hex1Cer | 0.7105 | 0.5870 | 0.8339 |
| Taurocholic acid | CerP | Hex1Cer | 0.7089 | 0.5857 | 0.8320 |
| CerP | CerPE | Hex2Cer | 0.7085 | 0.5849 | 0.8321 |
| Cholesterol sulfate | Taurocholic acid | Hex1Cer | 0.7065 | 0.5827 | 0.8304 |
| Glycocholic acid | Cholesterol sulfate | FA | 0.7034 | 0.5799 | 0.8269 |
| CerP | CL | Hex2Cer | 0.7026 | 0.5721 | 0.8331 |
| AcHexSiE | CerPE | GD1a | 0.7014 | 0.5964 | 0.8065 |
| CL | PG | ST | 0.6935 | 0.6064 | 0.7807 |
| CL | PG | ST(d18:1/20:2) | 0.6935 | 0.6064 | 0.7807 |
| CerP | Hex1Cer | Hex2Cer | 0.6884 | 0.5584 | 0.8185 |
| Glycocholic acid | Cholesterol sulfate | CerP | 0.6727 | 0.5428 | 0.8026 |
| Glycocholic acid | Cholesterol sulfate | Taurocholic acid | 0.6672 | 0.5401 | 0.7942 |
| Glycocholic acid | Cholesterol sulfate | GD1a | 0.6656 | 0.5396 | 0.7916 |

### D. REFERENCES

1. Golabi P, Fazel S, Otgonsuren M, Sayiner M, Locklear C T, Younossi Z M. Mortality assessment of patients with hepatocellular carcinoma according to underlying disease and treatment modalities. Medicine (Baltimore) 2017; 96:e5904.
2. Marrero J A. Surveillance for Hepatocellular Carcinoma. Clin Liver Dis 2020; 24:611-621.
3. Wolf E, Rich N E, Marrero J A, Parikh N D, Singal A G. Use of Hepatocellular Carcinoma Surveillance in Patients With Cirrhosis: A Systematic Review and Meta-Analysis. Hepatology 2021; 73:713-725.
4. Tzartzeva K, Obi J, Rich N E, Parikh N D, Marrero J A, Yopp A, et al. Surveillance Imaging and Alpha Fetoprotein for Early Detection of Hepatocellular Carcinoma in Patients With Cirrhosis: A Meta-analysis. Gastroenterology 2018; 154:1706-1718 e1701.
5. Kalluri R. The biology and function of exosomes in cancer. J Clin Invest 2016; 126:1208-1215.
6. Tang Z, Li D, Hou S, Zhu X. The cancer exosomes: Clinical implications, applications and challenges. Int J Cancer 2020; 146:2946-2959.
7. Ruivo C F, Adem B, Silva M, Melo S A. The Biology of Cancer Exosomes: Insights and New Perspectives. Cancer Res 2017; 77:6480-6488.
8. Othman N, Jamal R, Abu N. Cancer-Derived Exosomes as Effectors of Key Inflammation-Related Players. Front Immunol 2019; 10:2103.
9. Kalluri R, LeBleu V S. The biology, function, and biomedical applications of exosomes. Science 2020; 367.
10. Whiteside T L. Exosomes and tumor-mediated immune suppression. J Clin Invest 2016; 126:1216-1223.
11. Robbins P D, Dorronsoro A, Booker C N. Regulation of chronic inflammatory and immune processes by extracellular vesicles. J Clin Invest 2016; 126:1173-1180.
12. Kurywchak P, Tavormina J, Kalluri R. The emerging roles of exosomes in the modulation of immune responses in cancer. Genome Med 2018; 10:23.
13. Daassi D, Mahoney K M, Freeman G J. The importance of exosomal PDL1 in tumour immune evasion. Nat Rev Immunol 2020; 20:209-215.
14. Cheng N, Du D, Wang X, Liu D, Xu W, Luo Y, et al. Recent Advances in Biosensors for Detecting Cancer-Derived Exosomes. Trends Biotechnol 2019; 37:1236-1254.
15. Gulei D, Petrut B, Tigu A B, Onaciu A, Fischer-Fodor E, Atanasov A G, et al. Exosomes at a glance—common nominators for cancer hallmarks and novel diagnosis tools. Crit Rev Biochem Mol Biol 2018; 53:564-577.
16. Pathan M, Fonseka P, Chitti S V, Kang T, Sanwlani R, Van Deun J, et al. Vesiclepedia 2019: a compendium of RNA, proteins, lipids and metabolites in extracellular vesicles. Nucleic Acids Res 2019; 47:D516-D519.
17. Kosaka N, Yoshioka Y, Fujita Y, Ochiya T. Versatile roles of extracellular vesicles in cancer. J Clin Invest 2016; 126:1163-1172.
18. Li A, Zhang T, Zheng M, Liu Y, Chen Z. Exosomal proteins as potential markers of tumor diagnosis. J Hematol Oncol 2017; 10:175.
19. Melo S A, Luecke L B, Kahlert C, Fernandez A F, Gammon S T, Kaye J, et al. Glypican-1 identifies cancer exosomes and detects early pancreatic cancer. Nature 2015; 523:177-182.
20. Pocsfalvi G, Stanly C, Vilasi A, Fiume I, Capasso G, Turiak L, et al. Mass spectrometry of extracellular vesicles. Mass Spectrom Rev 2016; 35:3-21.
21. Luo X, An M, Cuneo K C, Lubman D M, Li L. High-Performance Chemical Isotope Labeling Liquid Chromatography Mass Spectrometry for Exosome Metabolomics. Anal Chem 2018; 90:8314-8319.
22. Penfornis P, Vallabhaneni K C, Whitt J, Pochampally R. Extracellular vesicles as carriers of microRNA, proteins and lipids in tumor microenvironment. Int J Cancer 2016; 138:14-21.
23. Skotland T, Sagini K, Sandvig K, Llorente A. An emerging focus on lipids in extracellular vesicles. Adv Drug Deliv Rev 2020; 159:308-321.
24. Yopp A C, Mansour J C, Beg M S, Arenas J, Trimmer C, Reddick M, et al. Establishment of a multidisciplinary hepatocellular carcinoma clinic is associated with improved clinical outcome. Ann Surg Oncol 2014; 21:1287-1295.
25. Rich N E, John B V, Parikh N D, Rowe I, Mehta N, Khatri G, et al. Hepatocellular Carcinoma Demonstrates Heterogeneous Growth Patterns in a Multicenter Cohort of Patients With Cirrhosis. Hepatology 2020; 72:1654-1665.
26. El-Serag H B, Kanwal F, Feng Z, Marrero J A, Khaderi S, Singal A G, et al. Risk Factors for Cirrhosis in Contemporary Hepatology Practices-Findings From the Texas Hepatocellular Carcinoma Consortium Cohort. Gastroenterology 2020; 159:376-377.
27. Heimbach J K, Kulik L M, Finn R S, Sirlin C B, Abecassis M M, Roberts L R, et al. AASLD guidelines for the treatment of hepatocellular carcinoma. Hepatology 2018; 67:358-380.
28. Thery C, Amigorena S, Raposo G, Clayton A. Isolation and characterization of exosomes from cell culture supernatants and biological fluids. Curr Protoc Cell Biol 2006; Chapter 3: Unit 3 22.
29. Liu X, Ser Z, Locasale J W. Development and quantitative evaluation of a high-resolution metabolomics technology. Anal Chem 2014; 86:2175-2184.
30. Yuan M, Breitkopf S B, Yang X, Asara J M. A positive/negative ion-switching, targeted mass spectrometry-based metabolomics platform for bodily fluids, cells, and fresh and fixed tissue. Nat Protoc 2012; 7:872-881.
31. Wishart D S, Feunang Y D, Marcu A, Guo A C, Liang K, Vazquez-Fresno R, et al. HMDB 4.0: the human metabolome database for 2018. Nucleic Acids Res 2018; 46:D608-D617.
32. Benjamini Y, Hochberg Y. Controlling the False Discovery Rate—a Practical and Powerful Approach to Multiple Testing. Journal of the Royal Statistical Society Series B-Statistical Methodology 1995; 57:289-300.
33. Firth D. Bias Reduction of Maximum-Likelihood-Estimates. Biometrika 1993; 80:27-38.
34. Nojima H, Freeman C M, Schuster R M, Japtok L, Kleuser B, Edwards M J, et al. Hepatocyte exosomes mediate liver repair and regeneration via sphingosine-1-phosphate. J Hepatol 2016; 64:60-68.
35. Wang R, Ding Q, Yaqoob U, de Assuncao T M, Verma V K, Hirsova P, et al. Exosome Adherence and Internalization by Hepatic Stellate Cells Triggers Sphingosine 1-Phosphate-dependent Migration. J Biol Chem 2015; 290:30684-30696.
36. Jiang Y, Tie C, Wang Y, Bian D, Liu M, Wang T, et al. Upregulation of Serum Sphingosine (d18:1)-1-P Potentially Contributes to Distinguish HCC Including AFP-Negative HCC From Cirrhosis. Front Oncol 2020; 10:1759.
37. Stepien M, Keski-Rahkonen P, Kiss A, Robinot N, Duarte-Salles T, Murphy N, et al. Metabolic perturbations prior to hepatocellular carcinoma diagnosis: Findings from a prospective observational cohort study. Int J Cancer 2021; 148:609-625.
38. Dasgupta S, Kumar V. Type II NKT cells: a distinct CDld-restricted immune regulatory NKT cell subset. Immunogenetics 2016; 68:665-676.
39. Maricic I, Sheng H, Marrero I, Seki E, Kisseleva T, Chaturvedi S, et al. Inhibition of type I natural killer T cells by retinoids or following sulfatide-mediated activation of type II natural killer T cells attenuates alcoholic liver disease in mice. Hepatology 2015; 61:1357-1369.
40. Shanbhag K, Mhetre A, Khandelwal N, Kamat S S. The Lysophosphatidylserines—An Emerging Class of Signalling Lysophospholipids. J Membr Biol 2020; 253:381-397.
41. Yanagida K, Valentine W J. Druggable Lysophospholipid Signaling Pathways. Adv Exp Med Biol 2020; 1274:137-176.
42. Xie G, Wang X, Huang F, Zhao A, Chen W, Yan J, et al. Dysregulated hepatic bile acids collaboratively promote liver carcinogenesis. Int J Cancer 2016; 139:1764-1775.
43. Kwan S Y, Jiao J, Qi J, Wang Y, Wei P, McCormick J B, et al. Bile Acid Changes Associated With Liver Fibrosis and Steatosis in the Mexican-American Population of South Texas. Hepatol Commun 2020; 4:555-568.
44. Petrick J L, Florio A A, Koshiol J, Pfeiffer R M, Yang B, Yu K, et al. Prediagnostic concentrations of circulating bile acids and hepatocellular carcinoma risk: REVEAL-HBV and HCV studies. Int J Cancer 2020; 147:2743-2753.
45. Loftfield E, Rothwell J A, Sinha R, Keski-Rahkonen P, Robinot N, Albanes D, et al. Prospective Investigation of Serum Metabolites, Coffee Drinking, Liver Cancer Incidence, and Liver Disease Mortality. J Natl Cancer Inst 2020; 112:286-294.
46. Chiu A P, Tschida B R, Sham T T, Lo L H, Moriarity B S, Li X X, et al. HBx-K130M/V131I Promotes Liver Cancer in Transgenic Mice via AKT/FOXO1 Signaling Pathway and Arachidonic Acid Metabolism. Mol Cancer Res 2019; 17:1582-1593.
47. Liu Z, Wang J, Liu L, Yuan H, Bu Y, Feng J, et al. Chronic ethanol consumption and HBV induce abnormal lipid metabolism through HBx/SWELL1/arachidonic acid signaling and activate Tregs in HBV-Tg mice. Theranostics 2020; 10:9249-9267.
48. Nie J, Lin B, Zhou M, Wu L, Zheng T. Role of ferroptosis in hepatocellular carcinoma. J Cancer Res Clin Oncol 2018; 144:2329-2337.
49. Li J, Cao F, Yin H L, Huang Z J, Lin Z T, Mao N, et al. Ferroptosis: past, present and future. Cell Death Dis 2020; 11:88.
50. Sun N, Lee Y T, Zhang R Y, Kao R, Teng P C, Yang Y, et al. Purification of HCC-specific extracellular vesicles on nanosubstrates for early HCC detection by digital scoring. Nat Commun 2020; 11:4489.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

1. A method of detecting hepatocellular carcinoma biomarkers in exosomes isolated from a sample, comprising detecting in the exosomes one or more hepatocellular carcinoma biomarkers,
wherein the one or more hepatocellular carcinoma biomarkers comprise one or more hepatocellular carcinoma biomarkers listed in any one or more of Table 1, Table 2, Table 3, Table 4, or Table 7A, any one or more two-way combinations of biomarkers listed in Table 4, Table 5, or Table 7B, any one or more three-way combinations of hepatocellular carcinoma biomarkers listed in Table 7C, or any combination of any thereof, and
wherein the sample is a blood sample obtained from a human subject.

2. The method of claim 1, wherein the hepatocellular carcinoma biomarkers comprise one or more of the lipid classes sphingosine (SPH), sulfatide (ST), or lysophosphatidylserine (LysoPS).

3. The method of claim 2, wherein the sphingosine lipid class comprises SPH(t18:0), the sulfatide lipid class comprises ST(d18:1/20:2), the lysophosphatidylserine lipid class comprises LysoPS(34:1), or any combination of any thereof.

4. The method of claim 1, wherein the hepatocellular carcinoma biomarkers comprise a lipid species selected from the group consisting of PC(18:1/24:2), PE(20:Op/20:3), and LysoPC(18:2).

5. The method of claim 1, wherein the hepatocellular carcinoma biomarkers further comprise alpha-fetoprotein.

6. The method of claim 1, wherein the subject is a patient previously diagnosed with liver cirrhosis, optionally wherein the liver cirrhosis is advanced liver cirrhosis.

7. (canceled)

8. The method of claim 1, wherein the detecting comprises detection by gas chromatography, mass spectroscopy, gas chromatography-mass spectrometry, or liquid chromatograph-mass spectrometry.

9. (canceled)

10. The method of claim 1, wherein 2, 3, 4, 5, 6, 7, 8, 9, 10, or more biomarkers are detected in the exosomes.

11. (canceled)

12. (canceled)

13. The method of claim 1, wherein the detecting comprises determining the concentration of the one or more biomarkers in the exosomes.

14. The method of claim 13, further comprising comparing the concentration of the one or more biomarkers in the exosomes isolated from the blood sample from the subject to a control level, wherein the control level corresponds to the concentration of the one or more biomarkers in exosomes isolated from a blood sample from a healthy individual without hepatocellular carcinoma.

15. The method of claim 14, further comprising treating the subject with an anti-hepatocellular carcinoma treatment based on a detection of a difference in the concentration of the one or more biomarkers in the exosomes isolated from the blood sample from the subject relative to the control level.

16. The method of claim 15, wherein the difference comprises an elevated level of SPH, a reduced level of ST, or an elevated level of LysoPS in the exosomes isolated from the blood sample from the subject relative to the control level, or any combination of any thereof.

17. The method of claim 1, wherein the subject is undergoing treatment for hepatocellular carcinoma, and wherein the sample comprises at least two samples obtained at different time points during the treatment.

18. The method of claim 1, wherein the subject is undergoing treatment for hepatocellular carcinoma, and wherein the sample comprises at least one sample obtained at a time point prior to start of the treatment, and at least one sample obtained at a time point subsequent to the start of the treatment.

19. The method of claim 17, wherein the treatment comprises one or more of a drug treatment, a radiation treatment, or a surgical treatment.

20. A method of generating a report containing information on results of detection of hepatocellular carcinoma biomarkers in exosomes, comprising:
detecting in the exosomes one or more hepatocellular carcinoma biomarkers; and,
generating the report,
wherein the one or more hepatocellular carcinoma biomarkers comprise one or more hepatocellular carcinoma biomarkers listed in any one or more of Table 1, Table 2, Table 3, Table 4, or Table 7A, any one or more two-way combinations of biomarkers listed in Table 4, Table 5, or Table 7B, any one or more three-way combinations of hepatocellular carcinoma biomarkers listed in Table 7C, or any combination of any thereof,
wherein the exosomes have been isolated from a blood sample obtained from a subject,
and wherein the report is useful for diagnosing hepatocellular carcinoma in the subject.

21. The method of claim 20, wherein the one or more hepatocellular carcinoma biomarkers are one or more of the lipid classes sphingosine (SPH), sulfatide (ST), or lysophosphatidylserine (LysoPS).

22. A system for detecting hepatocellular carcinoma biomarkers in exosomes, comprising a station for analyzing the exosomes by ultra-high resolution mass spectrometry to detect one or more hepatocellular carcinoma biomarkers in the exosomes,
wherein the one or more hepatocellular carcinoma biomarkers comprise one or more hepatocellular carcinoma biomarkers listed in any one or more of Table 1, Table 2, Table 3, Table 4, or Table 7A, any one or more two-way combinations of biomarkers listed in Table 4, Table 5, or Table 7B, any one or more three-way combinations of hepatocellular carcinoma biomarkers listed in Table 7C, or any combination of any thereof, and
wherein the exosomes have been isolated from a blood sample from a subject.

23. The method of claim 22, wherein the one or more hepatocellular carcinoma biomarkers are one or more of the lipid classes sphingosine (SPH), sulfatide (ST), or lysophosphatidylserine (LysoPS).

24. The system of claim 22, further comprising a station for generating a report containing information on results of the analyzing.
